(19) United States
(12) Patent Application Publication
(10) Pub. No.: US 2023/0070277 A1
(43) Pub. Date: Mar. 9, 2023

(54) METAL COMPLEX, AND COMPOSITION AND LIGHT EMITTING DEVICE CONTAINING THE SAME

(71) Applicant: Sumitomo Chemical Company, Limited, Chuo-ku, Tokyo (JP)

(72) Inventors: Shin SUZUKI, Tsukuba-shi, Ibaraki (JP); Motoaki USUI, Osaka-shi, Osaka (JP); Ryuji MATSUMOTO, Tsukuba-shi, Ibaraki (JP); Kazuei OHUCHI, Tsukuba-shi, Ibaraki (JP)

(21) Appl. No.: 17/780,359

(22) PCT Filed: Dec. 9, 2020
(86) PCT No.: PCT/JP2020/045823

§ 371 Date: May 26, 2022

(30) Foreign Application Priority Data
Dec. 27, 2019 (JP) 2019-239393

(51) Int. Cl. C07F 15/00; H01L 51/00

(52) U.S. Cl. CPC C07F 15/0033; H01L 51/0085

(57) ABSTRACT

A metal complex which is useful for production of a light emitting device showing improved luminance is provided. A composition containing the metal complex and a light emitting device containing the metal complex are also provided. The metal complex is represented by the following formula (0). The composition contains the metal complex and at least one material selected from the group consisting of a host material, a light emitting material other than the metal complex, an antioxidant and a solvent, and a light emitting device containing the metal complex.

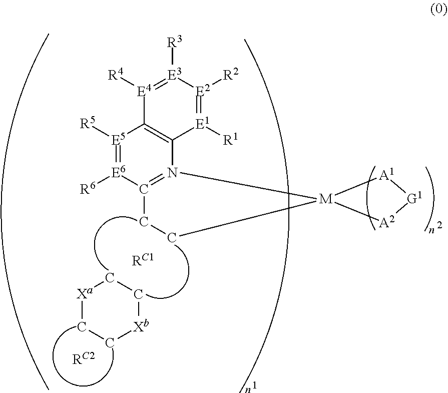

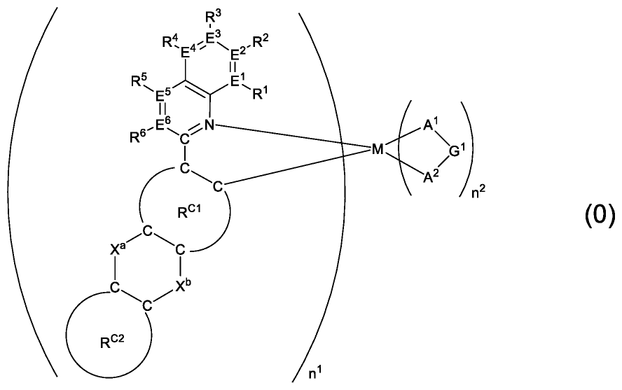

### FIELD OF THE INVENTION

The present invention relates to a metal complex, a composition containing the metal complex, and a light emitting device containing the metal complex.

### BACKGROUND ART

As the light emitting material used for a light emitting layer of a light emitting device, phosphorescent compounds showing light emission from the triplet excited state, and the like, are variously investigated. As such a phosphorescent compound, many metal complexes in which the central metal is a transition metal belonging to the 5th period or the 6th period have been studied. For example, Patent Document 1 suggests a metal complex having a fluorenylquinoline structure as a ligand (for example, a metal complex represented by the following formula).

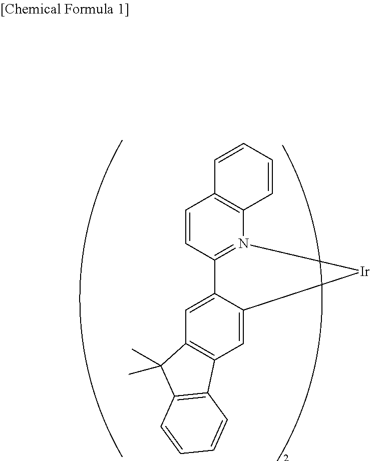

### PRIOR ART DOCUMENT

### Patent Document

Patent Document 1: Japanese Unexamined Patent Application Publication (JP-A) No. 2006-151888

### SUMMARY OF THE INVENTION

### Problem to be Solved by the Invention

However, a light emitting device fabricated using the above-described metal complex was not necessarily sufficient in luminescence lifetime.

Then, the present invention has an object of providing a metal complex which is useful for production of a light emitting device showing improved luminescence lifetime. Further, the present invention has an object of providing a composition containing the metal complex, and a light emitting device containing the metal complex.

### Means for Solving the Problem

The present invention provides the following [1] to [12].

[1]

A metal complex represented by the following formula (0):

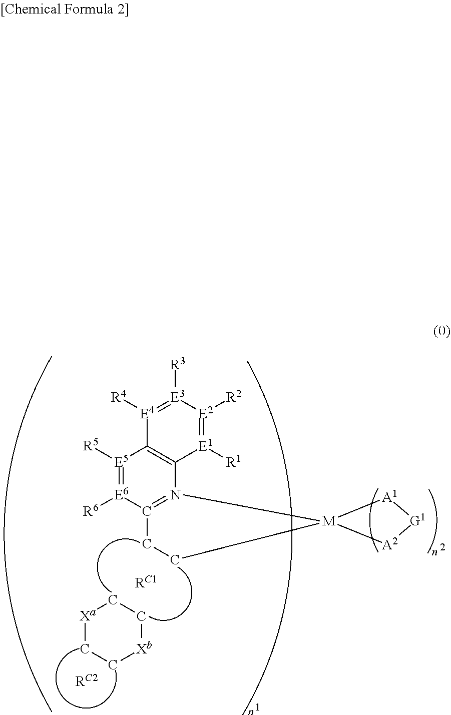

[wherein,

M represents a rhodium atom, a palladium atom, an iridium atom or a platinum atom;

n¹ represents 1, 2 or 3; n² represents 0, 1 or 2; n¹+n² is 3 when M is a rhodium atom or an iridium atom, while n¹+n² is 2 when M is a platinum atom or a palladium atom;

when n¹ is 2 or 3, ligands of which number is defined by index n¹ may be the same or different; and when n² is 2, ligands of which number is defined by index n² may be the same or different;

and in respective ligands or one ligand of which number is defined by index n¹, Ring R^{C1} and Ring R^{C2} each independently represent an aromatic hydrocarbon ring or an aromatic heterocyclic ring, and these rings optionally have a substituent, when a plurality of the substituents are present, they may be combined together to form a ring together with atoms to which they are attached;

and in respective ligands or one ligand of which number is defined by index n¹, one of X^{a} and X^{b} is a single bond, the other is a group represented by —C(R^{Xa})₂—, a group represented by —C(R^{Xa})₂—C(R^{Xa})₂— or a group represented by —C(R^{Xa})═C(R^{Xa})—; R^{Xa} represents a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent, when a plurality of the substituents are present, they may be combined together to form a ring together with atoms to which they are attached, a plurality of R^{Xa} may be the same or different and may be combined together to form a ring together with carbon atoms to which they are attached;

and in respective ligands or one ligand of which number is defined by index n¹, E¹, E², E³, E⁴, E⁵, and E⁶ each independently represent a nitrogen atom or a carbon atom, and at least E¹ and E², E² and E³, E³ and E⁴, E⁴ and E⁵, or E⁵ and E⁶, are carbon atoms; provided that, when E¹ is a nitrogen atom, R¹ is not present; when E² is a nitrogen atom, R² is not present; when E³ is a nitrogen atom, R³ is not present; when E⁴ is a nitrogen atom, R⁴ is not present; when E⁵ is a nitrogen atom, R⁵ is not present; when E⁶ is a nitrogen atom, R⁶ is not present; and in respective ligands or one ligand of which number is defined by index n¹, the combination of one of R¹ and R², R² and R³, R³ and R⁴, R⁴ and R⁵, and R⁵ and R⁶ is integrated to form a group represented by the following formula (P); R¹, R², R³, R⁴, R⁵ and R⁶ not forming a group represented by the following formula (P) each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent, when a plurality of the substituents are present, they may be combined together to form a ring together with atoms to which they are attached; and in respective ligands or one ligand of which number is defined by index n¹, when a plurality of R¹, R², R³, R⁴, R⁵ and R⁶ not forming a group represented by the following formula (P) are present, they may be the same or different at each occurrence, and may be combined together to form a ring together with carbon atoms to which they are attached;

and in respective ligands or one ligand of which number is defined by index n², A¹-G¹-A² represents an anionic bidentate ligand, G¹ represents an atomic group constituting a bidentate ligand together with A¹ and A², A¹ and A² each independently represent a carbon atom, an oxygen atom or a nitrogen atom, or an atomic group having them, and these atomic groups may be ring-constituent atomic groups].

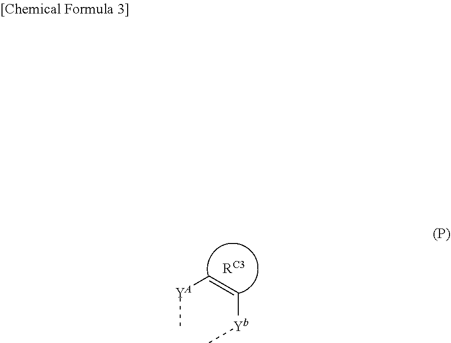

[wherein,

the dotted line denotes a bond to E¹, E², E³, E⁴, E⁵ or E⁶;

Ring R^{C3} represents an aromatic hydrocarbon ring or an aromatic heterocyclic ring, and these rings optionally have a substituent, when a plurality of the substituents are present, they may be combined together to form a ring together with atoms to which they are attached;

one of Y^{a} and Y^{b} is a single bond, and the other is a group represented by —C(R^{Ya})₂—, R^{Ya} represents a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent, when a plurality of the substituents are present, they may be combined together to form a ring together with atoms to which they are attached; a plurality of R^{Ya} may be the same or different and may be combined together to form a ring together with carbon atoms to which they are attached].

[2] The metal complex according to [1], wherein in respective ligands or one ligand of which number is defined by index n¹ in the above-described formula (0), R² and R³ are integrated to form a group represented by the above-described formula (P).

[3] The metal complex according to [1] or [2], wherein the above-described formula (P) is represented by the following formula (P′):

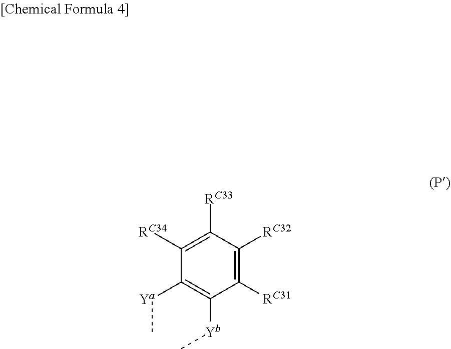

[wherein,

the dotted line denotes a bond to E¹, E², E³, E⁴, E⁵ or E⁶;

Y^{a} and Y^{b} represent the same meaning as for Y^{a} and Y^{b} in the above-described formula (P), respectively;

R^{C31}, R^{C32}, R^{C33} and R^{C34} each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent; R^{C31} and R^{C32}, R^{C32} and R^{C33}, and R^{C33} and R^{C34} each may be combined together to form a ring together with atoms to which they are attached].

[4] The metal complex according to [3], wherein the metal complex represented by the above-described formula (0) is a metal complex represented by the following formula (2-A1), the following formula (2-B1) or the following formula (2-C1):

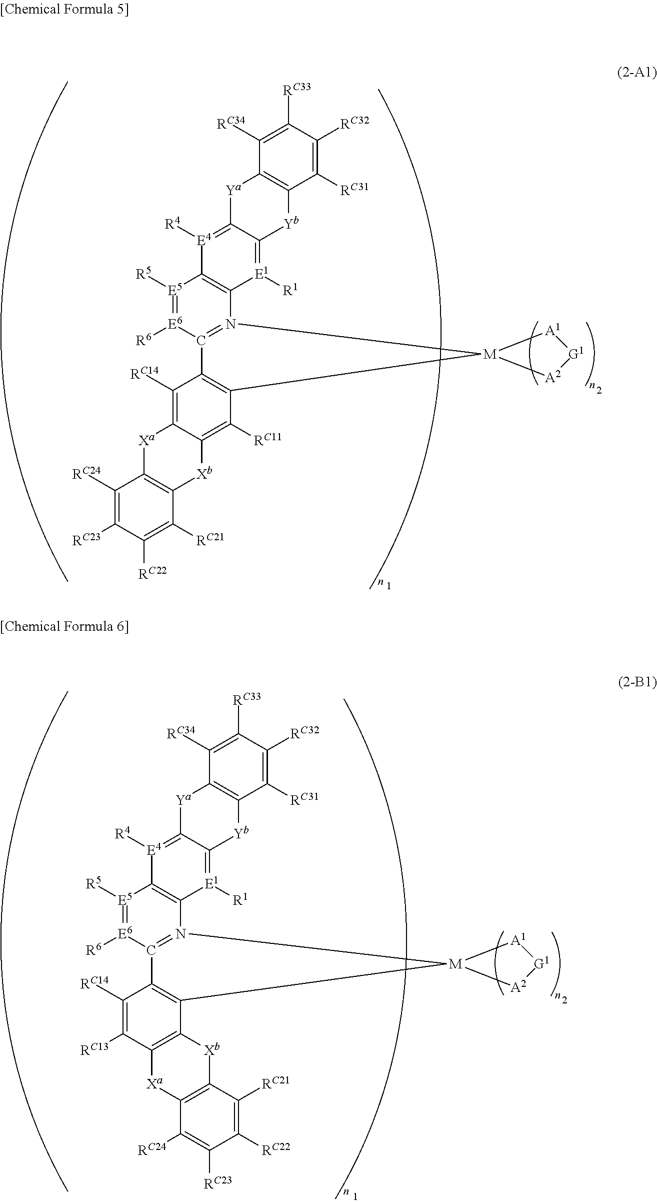

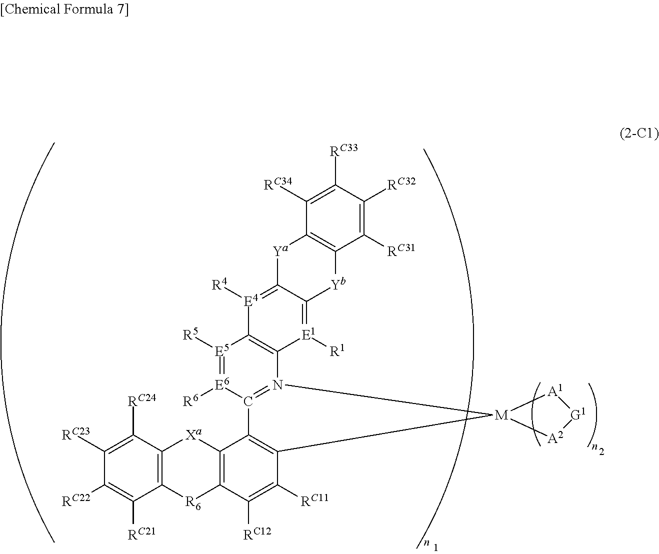

[In the formula (2-A1), the formula (2-B1), and the formula (2-C1),

M, n¹, n², X^{a}, X^{b}, E¹, E⁴, E⁵, E⁶, R¹, R⁴, R⁵, R⁶, and A¹-G¹-A² represent the same meaning as for M, n¹, n², X^{a}, X^{b}, E¹, E⁴, E⁵, E⁶, R¹, R⁴, R⁵, R⁶, and A¹-G¹-A² in the above-described formula (0), respectively; Y^{a} and Y^{b} represent the same meaning as for Y^{a} and Y^{b} in the above-described formula (P), respectively; R^{C31}, R^{C32}, R^{C33}, and R^{C34} represent the same meaning as for R^{C31}, R^{C32}, R^{C33}, and R^{C34} in the above-described formula (P′), respectively;

R^{C11} and R^{C14} in the formula (2-A1), R^{C13} and R^{C14} in the formula (2-B1), and R^{C11} and R^{C12} in the formula (2-C1) each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent;

R^{C21}, R^{C22}, R^{C23} and R^{C24} each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent; and in respective ligands or one ligand of which number is defined by index n¹, R^{C11} and R^{C12}, R^{C13} and R^{C14}, R^{C21} and R^{C22}, R^{C22} and R^{C23}, and R^{C23} and R^{C24} each may be combined together to form a ring together with atoms to which they are attached].

[5] The metal complex according to [4], wherein the metal complex represented by the above-described formula (2-A1) is a metal complex represented by the following formula (2-A1-1):

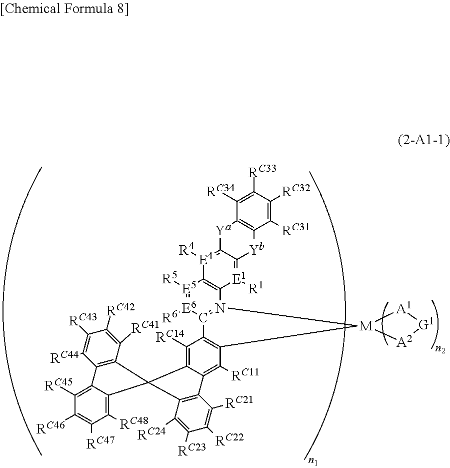

[wherein,

M, n¹, n², E¹, E⁴, E⁵, E⁶, R¹, R⁴, R⁵, R⁶, and A¹-G¹-A² represent the same meaning as for M, n¹, n², E¹, E⁴, E⁵, E⁶, R¹, R⁴, R⁵, R⁶, and A¹-G¹-A² in the above-described formula (0), respectively; Y^{a} and Y^{b} represent the same meaning as for Y^{a} and Y^{b} in the above-described formula (P), respectively; R^{C31}, R^{C32}, R^{C33}, and R^{C34} represent the same meaning as for R^{C31}, R^{C32}, R^{C33}, and R^{C34} in the above-described formula (P′), respectively; R^{C11}, R^{C14}, R^{C21}, R^{C22}, R^{C23}, and R^{C24} represent the same meaning as for R^{C11}, R^{C14}, R^{C21}, R^{C22}, R^{C23}, and R^{C24} in the above-described formula (2-A1), respectively;

R^{C41}, R^{C42}, R^{C43}, R^{C44}, R^{C45}, R^{C46}, R^{C47} and R^{C48} each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent; and in respective ligands or one ligand of which number is defined by index n¹, R^{C41} and R^{C42}, R^{C42} and R^{C43}, R^{C43} and R^{C44}, R^{C45} and R^{C46}, R^{C46} and R^{C47}, and R^{C47} and R^{C48} each may be combined together to form a ring together with atoms to which they are attached].

[6] The metal complex according to any one of [1] to [5], wherein the above-described Ring R^{C1}, the above-described Ring R^{C2} or the above-described Ring R^{C3} has a group represented by the following formula (D-A), the following formula (D-B) or the following formula (D-C) as a substituent:

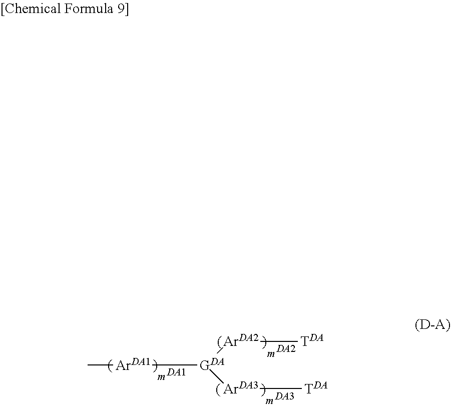

[wherein,

m^{DA1}, m^{DA2} and m^{DA3} each independently represent an integer of 0 or more;

G^{DA} represents a nitrogen atom, an aromatic hydrocarbon group or a heterocyclic ring group, and these groups optionally have a substituent;

Ar^{DA1}, Ar^{DA2} and Ar^{DA3} each independently represent an arylene group or a divalent heterocyclic ring group, and these groups optionally have a substituent; when a plurality of Ar^{DA1}, Ar^{DA2} and Ar^{DA3} are present, they may be the same or different at each occurrence;

T^{DA} represents an aryl group or a monovalent heterocyclic ring group, and these groups optionally have a substituent; a plurality of T^{DA} may be the same or different].

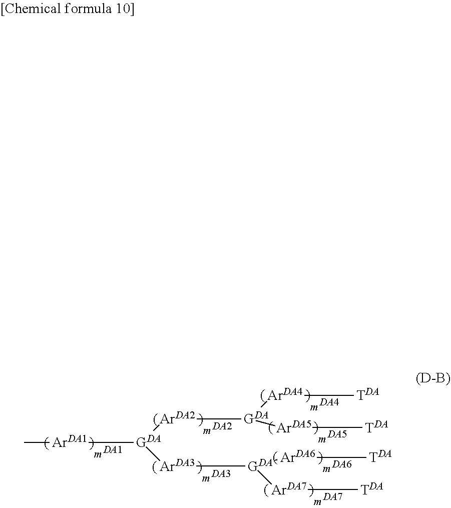

[wherein,

m^{DA1}, m^{DA2} m^{DA3} m^{DA4} m^{DA5}, m^{DA6} and m^{DA7} each independently represent an integer of 0 or more;

G^{DA} represents a nitrogen atom, an aromatic hydrocarbon group or a heterocyclic ring group, and these groups optionally have a substituent; a plurality of G^{DA} may be the same or different;

Ar^{DA1}, Ar^{DA2}, Ar^{DA3}, Ar^{DA4}, Ar^{DA5}, Ar^{DA6} and Ar^{DA7} each independently represent an arylene group or a divalent heterocyclic ring group, and these groups optionally have a substituent. When a plurality of Ar^{DA1}, Ar^{DA2}, Ar^{DA3}, Ar^{DA4}, Ar^{DA5}, Ar^{DA6} and Ar^{DA7} are present, they may be the same or different at each occurrence;

T^{DA} represents an aryl group or a monovalent heterocyclic ring group, and these groups optionally have a substituent; a plurality of T^{DA} may be the same or different].

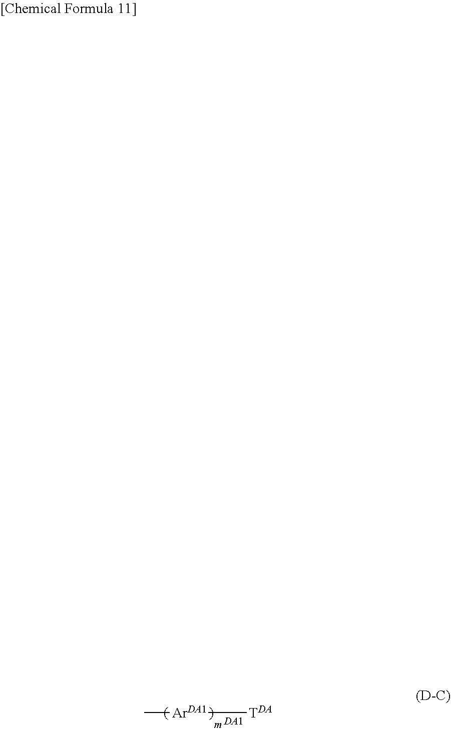

[wherein,

m^{DA1} represents an integer of 0 or more;

Ar^{DA1} represents an arylene group or a divalent heterocyclic ring group, and these groups optionally have a substituent; when a plurality of Ar^{DA1} are present, they may be the same or different;

T^{DA} represents an aryl group or a monovalent heterocyclic ring group, and these groups optionally have a substituent].

[7] The metal complex according to any one of [1] to [6], wherein R¹, R², R³, R⁴, R⁵ or R⁶ not forming a group represented by the above-described formula (P) is a group represented by the above-described formula (D-A), the above-described formula (D-B) or the above-described formula (D-C).

[8] The metal complex according to [7], wherein R⁴ or R⁵ not forming a group represented by the above-described formula (P) is a group represented by the above-described formula (D-A), the above-described formula (D-B) or the above-described formula (D-C).

[9] The metal complex according to any one of [1] to [8], wherein the above-described M is an iridium atom, and the above-described n¹ is 3.

[10] A composition comprising

the metal complex as described in any one of [1] to [9], and

at least one material selected from the group consisting of a host material, a light emitting material other than the above-described metal complex, an antioxidant and a solvent.

[11] The composition according to [10], wherein the above-described host material contains at least one of a low molecular compound represented by the following formula (H-1) and a polymer compound containing a constitutional unit represented by the following formula (Y):

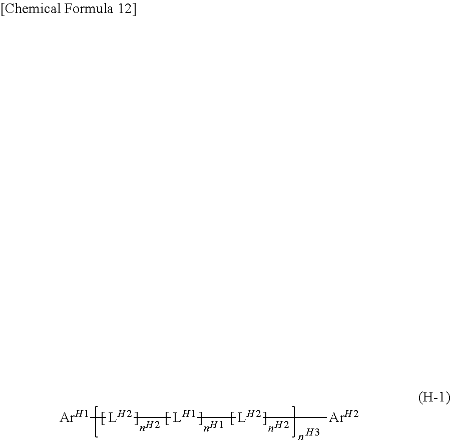

[wherein,

Ar^{H1} and Ar^{H2} each independently represent an aryl group or a monovalent heterocyclic ring group, and these groups optionally have a substituent;

n^{H1} and n^{H2} each independently represent 0 or 1; when a plurality of n^{H1} are present, they may be the same or different; when a plurality of n^{H2} are present, the plurality of n^{H2} may be the same or different;

n^{H3} represents an integer of 0 or more;

L^{H1} represents an arylene group, a divalent heterocyclic ring group, or a group represented by —[C(R^{H11})₂]n^{H11}-, and these groups optionally have a substituent, when a plurality of L^{H1} are present, they may be the same or different, n^{H11} represents an integer of 1 or more and 10 or less; R^{H11} represents a hydrogen atom, an alkyl group, a cycloalkyl group, an alkoxy group, a cycloalkoxy group, an aryl group or a monovalent heterocyclic ring group, and these groups optionally have a substituent, a plurality of R^{H11} may be the same or different and may be combined together to form a ring together with carbon atoms to which they are attached;

L^{H2} represents a group represented by —N(-L^{H21}-R^{H21})—; when a plurality of L^{H2} are present, the plurality of L^{H2} may be the same or different; L^{H21} represents a single bond, an arylene group or a divalent heterocyclic ring group, and these groups optionally have a substituent, R^{H21} represents a hydrogen atom, an alkyl group, a cycloalkyl group, an aryl group or a monovalent heterocyclic ring group, and these groups optionally have a substituent].

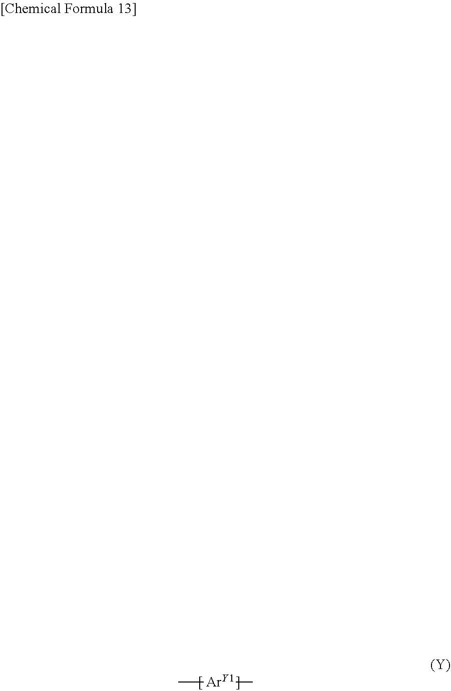

[wherein, Ar^{Y1} represents an arylene group, a divalent heterocyclic ring group, or a divalent group in which an arylene group and a divalent heterocyclic ring group are bonded directly, and these groups optionally have a substituent].

[12] A light emitting device comprising the metal complex as described in any one of [1] to [9].

### Effect of the Invention

According to the present invention, a metal complex which is useful for production of a light emitting device showing improved luminescence lifetime can be provided. Further, according to the present invention, a composition containing the metal complex and a light emitting device containing the metal complex can be provided.

### MODES FOR CARRYING OUT THE INVENTION

Suitable embodiments of the present invention will be illustrated in detail below

### Explanation of Common Terms

Terms commonly used in the present specification have the following meanings unless otherwise stated.

Me represents a methyl group, Et represents an ethyl group, Bu represents a butyl group, i-Pr represents an isopropyl group, and t-Bu represents a tert-butyl group.

A hydrogen atom may be a deuterated hydrogen atom or a light hydrogen atom.

In the formula representing a metal complex, the solid line representing a bond with a central metal means a covalent bond or a coordination bond.

“The low molecular compound” means a compound not having molecular weight distribution and having a molecular weight of 1×10⁴ or less.

“The polymer compound” means a polymer having molecular weight distribution, and having a polystyrene-equivalent number-average molecular weight of 1×10³ or more (for example, 1×10³ to 1×10⁸).

“The constitutional unit” means a unit occurring once or more times in a polymer compound. The constitutional unit occurring twice or more times in a polymer compound is in general referred to also as “repeating unit”.

The polymer compound may be any of a block copolymer, a random copolymer, an alternative copolymer or a graft copolymer, or may also be in other form.

It is preferable that the end group of the polymer compound is a stable group, since if a polymerization active group remains intact, there is a possibility of lowering of light emitting property or luminescence lifetime when the polymer compound is used for fabrication of a light emitting device. The end group of the polymer compound is preferably a group conjugatively bonded to the main chain, and is, for example, an aryl group or a monovalent heterocyclic ring group bonding to the main chain of the polymer compound via a carbon-carbon bond.

“The alkyl group” may be any of linear or branched. The number of carbon atoms of the linear alkyl group, not including the number of carbon atoms of the substituent, is usually 1 to 50, preferably 3 to 30, and more preferably 4 to 20. The number of carbon atoms of the branched alkyl group, not including the number of carbon atoms of the substituent, is usually 3 to 50, preferably 3 to 30, and more preferably 4 to 20.

The alkyl group optionally has a substituent. The alkyl group includes, for example, a methyl group, an ethyl group, a propyl group, an isopropyl group, a butyl group, a 2-butyl group, an isobutyl group, a tert-butyl group, a pentyl group, an isoamyl group, a 2-ethylbutyl group, a hexyl group, a heptyl group, an octyl group, a 2-ethylhexyl group, a 3-propylheptyl group, a decyl group, a 3,7-dimethyloctyl group, a 2-ethyloctyl group, a 2-hexyldecyl group and a dodecyl group. Further, the alkyl group may be a group obtained by substituting a part or all of hydrogen atoms in these groups with a cycloalkyl group, an alkoxy group, a cycloalkoxy group, an aryl group, and/or a fluorine atom, and the like. Such an alkyl group includes, for example, a trifluoromethyl group, a pentafluoroethyl group, a perfluorobutyl group, a perfluorohexyl group, a perfluorooctyl group, a 3-phenylpropyl group, a 3-(4-methylphenyl)propyl group, a 3-(3,5-di-hexylphenyl)propyl group, and a 6-ethyloxyhexyl group.

The number of carbon atoms of “the cycloalkyl group”, not including the number of carbon atoms of the substituent, is usually 3 to 50, preferably 3 to 30, and more preferably 4 to 20. The cycloalkyl group optionally has a substituent. The cycloalkyl group includes, for example, a cyclohexyl group, a cyclohexylmethyl group, and a cyclohexylethyl group.

“The aryl group” means an atomic group remaining after removing from an aromatic hydrocarbon one hydrogen atom bonding directly to a carbon atom constituting the ring. The number of carbon atoms of the aryl group, not including the number of carbon atoms of the substituent, is usually 6 to 60, preferably 6 to 20, and more preferably 6 to 10.

The aryl group optionally has a substituent. The aryl group includes, for example, a phenyl group, a 1-naphthyl group, a 2-naphthyl group, a 1-anthracenyl group, a 2-anthracenyl group, a 9-anthracenyl group, a 1-pyrenyl group, a 2-pyrenyl group, a 4-pyrenyl group, a 2-fluorenyl group, a 3-fluorenyl group, a 4-fluorenyl group, a 2-phenylphenyl group, a 3-phenylphenyl group, and a 4-phenylphenyl group. Further, the aryl group may be a group obtained by substituting a part or all of hydrogen atoms in these groups with an alkyl group, a cycloalkyl group, an alkoxy group, a cycloalkoxy group, an aryl group, and/or a fluorine atom, and the like.

“The alkoxy group” may be any of linear or branched. The number of carbon atoms of the linear alkoxy group, not including the number of carbon atoms of the substituent, is usually 1 to 40, and preferably 4 to 10. The number of carbon atoms of the branched alkoxy group, not including the number of carbon atoms of the substituent, is usually 3 to 40, and preferably 4 to 10.

The alkoxy group optionally has a substituent. The alkoxy group includes, for example, a methoxy group, an ethoxy group, a propyloxy group, an isopropyloxy group, a butyloxy group, an isobutyloxy group, a tert-butyloxy group, a pentyloxy group, a hexyloxy group, a heptyloxy group, an octyloxy group, a 2-ethylhexyloxy group, a nonyloxy group, a decyloxy group, a 3,7-dimethyloctyloxy group, and a lauryloxy group. Further, the alkoxy group may be a group obtained by substituting a part or all of hydrogen atoms in these groups with a cycloalkyl group, an alkoxy group, a cycloalkoxy group, an aryl group, and/or a fluorine atom, and the like.

The number of carbon atoms of “the cycloalkoxy group”, not including the number of carbon atoms of the substituent, is usually 3 to 40, and preferably 4 to 10.

The cycloalkoxy group optionally has a substituent. The cycloalkoxy group includes, for example, a cyclohexyloxy group.

The number of carbon atoms of “the aryloxy group”, not including the number of carbon atoms of the substituent, is usually 6 to 60, and preferably 6 to 48.

The aryloxy group optionally has a substituent. The aryloxy group includes, for example, a phenoxy group, a 1-naphthyloxy group, a 2-naphthyloxy group, a 1-anthracenyloxy group, a 9-anthracenyloxy group, and a 1-pyrenyloxy group. Further, the aryloxy group may be a group obtained by substituting a part or all of hydrogen atoms in these groups with an alkyl group, a cycloalkyl group, an alkoxy group, a cycloalkoxy group, and/or a fluorine atom, and the like.

“The p-valent heterocyclic ring group” (p represents an integer of 1 or more) means an atomic group remaining after removing from a heterocyclic compound p hydrogen atoms among hydrogen atoms bonding directly to carbon atoms or hetero atoms constituting the ring. Of the p-valent heterocyclic ring groups, “a p-valent aromatic heterocyclic ring group” which is an atomic group remaining after removing from an aromatic heterocyclic compound p hydrogen atoms among hydrogen atoms bonding directly to carbon atoms or hetero atoms constituting the ring is preferred.

“The aromatic heterocyclic compound” means a compound in which the heterocyclic ring itself shows aromaticity such as oxadiazole, thiadiazole, thiazole, oxazole, thiophene, pyrrole, phosphole, furan, pyridine, pyrazine, pyrimidine, triazine, pyridazine, quinoline, isoquinoline, carbazole, dibenzophosphole, and the like, and a compound in which an aromatic ring is condensed to a heterocyclic ring even if the heterocyclic ring itself shows no aromaticity such as phenoxazine, phenothiazine, dibenzoborole, dibenzosilole, benzopyran, and the like.

The number of carbon atoms of the monovalent heterocyclic ring group, not including the number of carbon atoms of the substituent, is usually 2 to 60, and preferably 4 to 20.

The monovalent heterocyclic ring group optionally has a substituent. The monovalent heterocyclic ring group includes, for example, a thienyl group, a pyrrolyl group, a furyl group, a pyridinyl group, a piperidinyl group, a quinolinyl group, an isoquinolinyl group, a pyrimidinyl group, and a triazinyl group. Further, the monovalent heterocyclic ring group may be a group obtained by substituting a part or all of hydrogen atoms in these groups with an alkyl group, a cycloalkyl group, an alkoxy group, and/or a cycloalkoxy group, and the like.

“The halogen atom” denotes a fluorine atom, a chlorine atom, a bromine atom or an iodine atom.

“The amino group” optionally has a substituent, and is preferably a substituted amino group (preferably a secondary amino group or a tertiary amino group, and more preferably a tertiary amino group). As the substituent which an amino group has, an alkyl group, a cycloalkyl group, an aryl group or a monovalent heterocyclic ring group is preferred. When a plurality of the substituents which an amino group has are present, they may be the same or different and may be combined together to form a ring together with a nitrogen atom to which they are attached.

The substituted amino group includes, for example, a dialkylamino group (for example, a dimethylamino group, a diethylamino group), a dicycloalkylamino group, a diarylamino group (for example, a diphenylamino group), a bis(4-methylphenyl)amino group, a bis(4-tert-butylphenyl)amino group, and a bis(3,5-di-tert-butylphenyl)amino group.

“The alkenyl group” may be any of linear or branched. The number of carbon atoms of the linear alkenyl group, not including the number of carbon atoms of the substituent, is usually 2 to 30, and preferably 3 to 20. The number of carbon atoms of the branched alkenyl group, not including the number of carbon atoms of the substituent, is usually 3 to 30, and preferably 4 to 20.

The number of carbon atoms of “the cycloalkenyl group”, not including the number of carbon atoms of the substituent, is usually 3 to 30, and preferably 4 to 20.

The alkenyl group and the cycloalkenyl group optionally have a substituent. The alkenyl group includes, for example, a vinyl group, a 1-propenyl group, a 2-propenyl group, a 2-butenyl group, a 3-butenyl group, a 3-pentenyl group, a 4-pentenyl group, a 1-hexenyl group, a 5-hexenyl group and a 7-octenyl group, and a group obtained by substituting a part or all of hydrogen atoms in these groups with a substituent. The cycloalkenyl group includes, for example, a cyclohexenyl group, a cyclohexadienyl group, a cyclooctatrienyl group and a norbornylenyl group, and a group obtained by substituting a part or all of hydrogen atoms in these groups with a substituent.

“The alkynyl group” may be any of linear or branched. The carbon atoms of the linear alkynyl group, not including carbon atoms of the substituent, is usually 2 to 20, and preferably 3 to 20. The number of carbon atoms of the branched alkynyl group, not including carbon atoms of the substituent, is usually 4 to 30, and preferably 4 to 20.

The carbon atoms of “the cycloalkynyl group”, not including carbon atoms of the substituent, is usually 4 to 30, and preferably 4 to 20.

The alkynyl group and the cycloalkynyl group optionally have a substituent. The alkynyl group includes, for example, an ethynyl group, a 1-propynyl group, a 2-propynyl group, a 2-butynyl group, a 3-butynyl group, a 3-pentynyl group, a 4-pentynyl group, a 1-hexynyl group and a 5-hexynyl group, and a group obtained by substituting a part or all of hydrogen atoms in these groups with a substituent. The cycloalkynyl group includes, for example, a cyclooctynyl group, and a group obtained by substituting a part or all of hydrogen atoms in these groups with a substituent.

“The arylene group” means an atomic group remaining after removing from an aromatic hydrocarbon two hydrogen atoms bonding directly to carbon atoms constituting the ring. The number of carbon atoms of the arylene group, not including the number of carbon atoms of the substituent, is usually 6 to 60, preferably 6 to 30, and more preferably 6 to 18.

The arylene group optionally has a substituent. The arylene group includes, for example, a phenylene group, a naphthalenediyl group, an anthracenediyl group, a phenanthrenediyl group, a dihydrophenanthrenediyl group, a naphthacenediyl group, a fluorenediyl group, a pyrenediyl group, a perylenediyl group and a chrysenediyl group, and a group obtained by substituting a part or all of hydrogen atoms in these groups with a substituent. The arylene group is preferably a group represented by the following formula (A-1) to formula (A-20). The arylene group includes groups in which multiple of these groups are bonded.

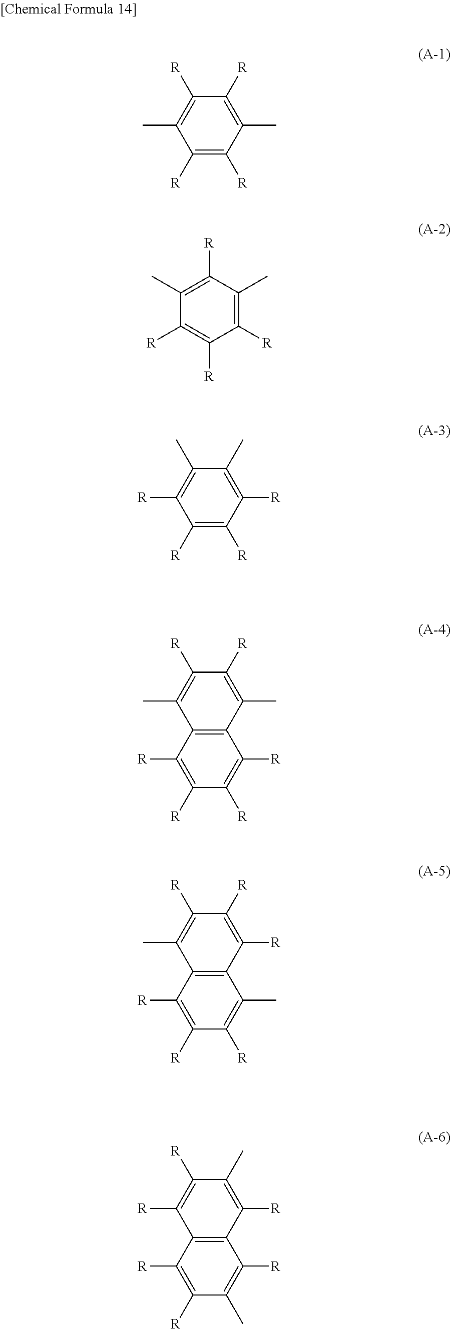

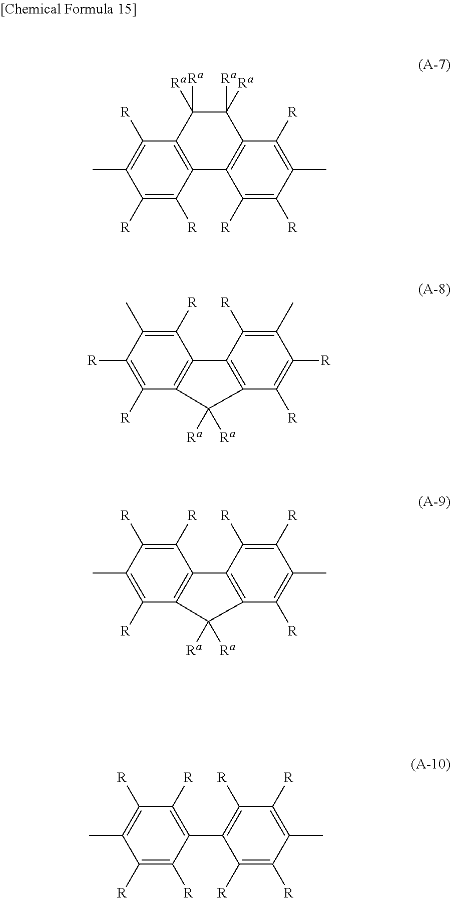

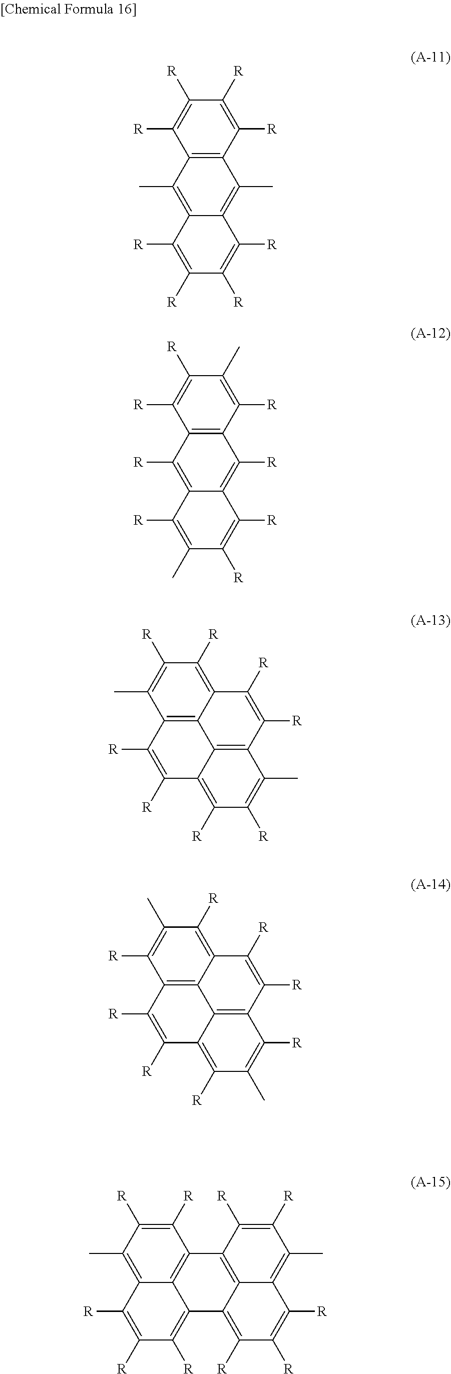

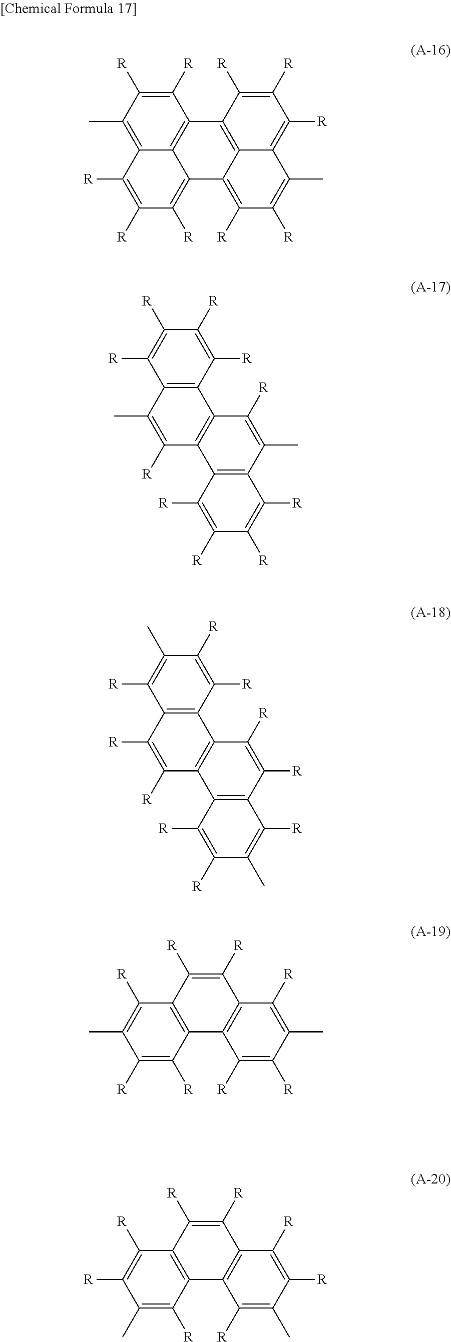

In the above-described formula (A-1) to formula (A-20), R and R^{a} each independently represent a hydrogen atom, an alkyl group, a cycloalkyl group, an aryl group or a monovalent heterocyclic ring group. A plurality of R and R^{a} each may be the same or different, and the plurality of R^{a} may be combined together to form a ring together with atoms to which they are attached.

The number of carbon atoms of the divalent heterocyclic ring group, not including the number of carbon atoms of the substituent, is usually 2 to 60, preferably 3 to 20, and more preferably 4 to 15.

The divalent heterocyclic ring group optionally has a substituent. The divalent heterocyclic ring group includes, for example, divalent groups obtained by removing from pyridine, diazabenzene, triazine, azanaphthalene, diazanaphthalene, carbazole, dibenzofuran, dibenzothiophene, dibenzosilole, phenoxazine, phenothiazine, acridine, dihydroacridine, furan, thiophene, azole, diazole or triazole two hydrogen atoms among hydrogen atoms bonding directly to carbon atoms or hetero atoms constituting the ring. Further, the divalent heterocyclic ring group may be a group obtained by substituting a part or all of hydrogen atoms in these groups with a substituent. The divalent heterocyclic ring group is preferably a group represented by the following formula (AA-1) to formula (AA-34). The divalent heterocyclic ring group includes groups in which multiple of these groups are bonded.

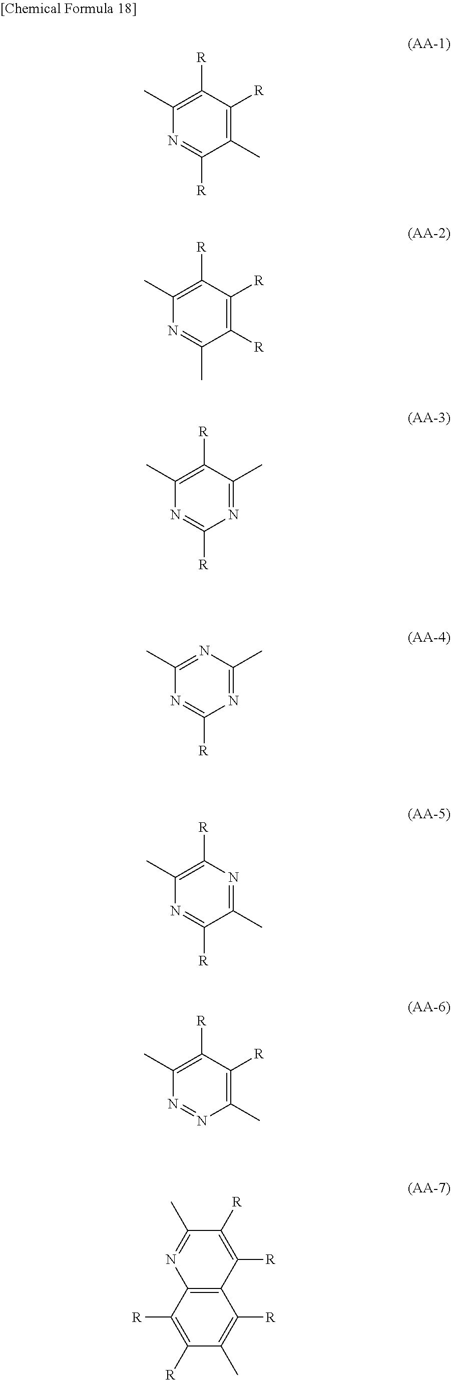

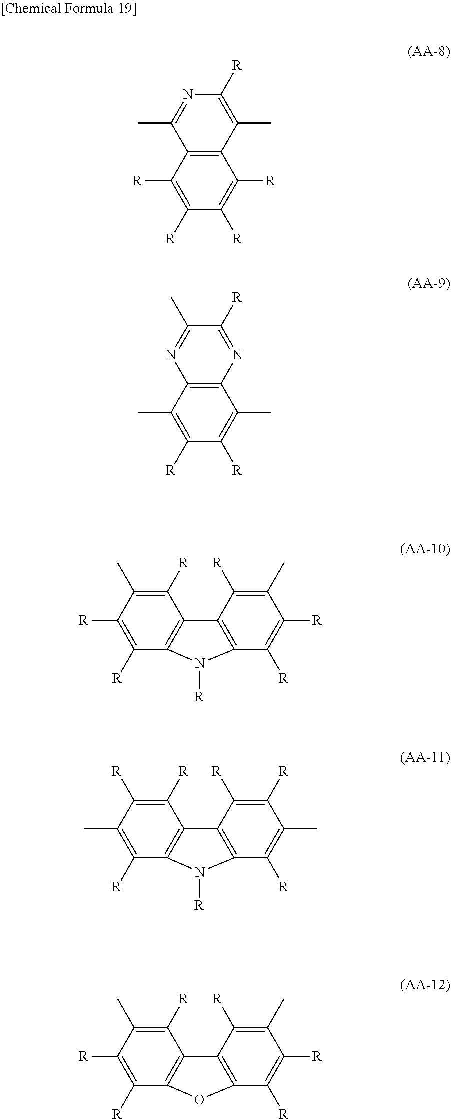

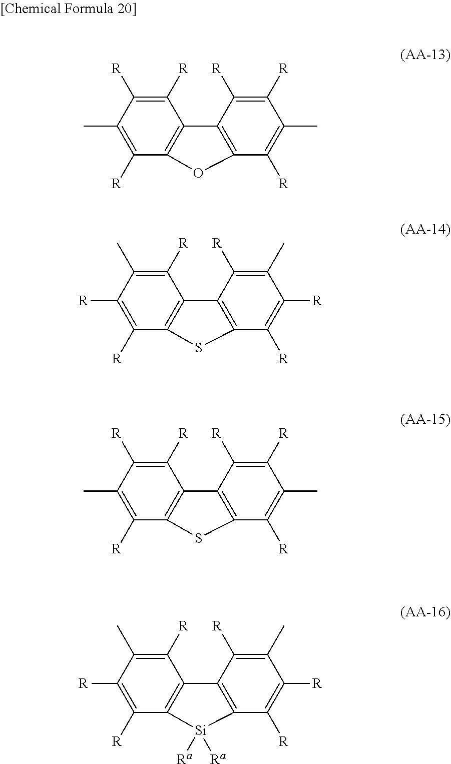

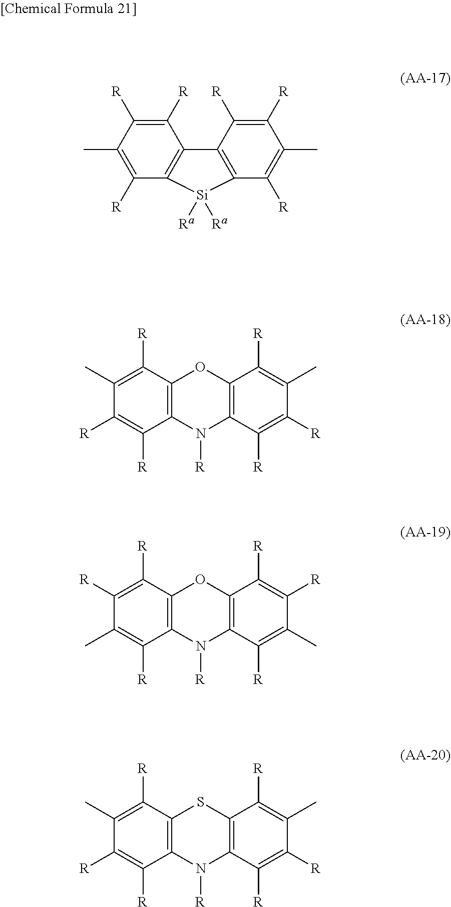

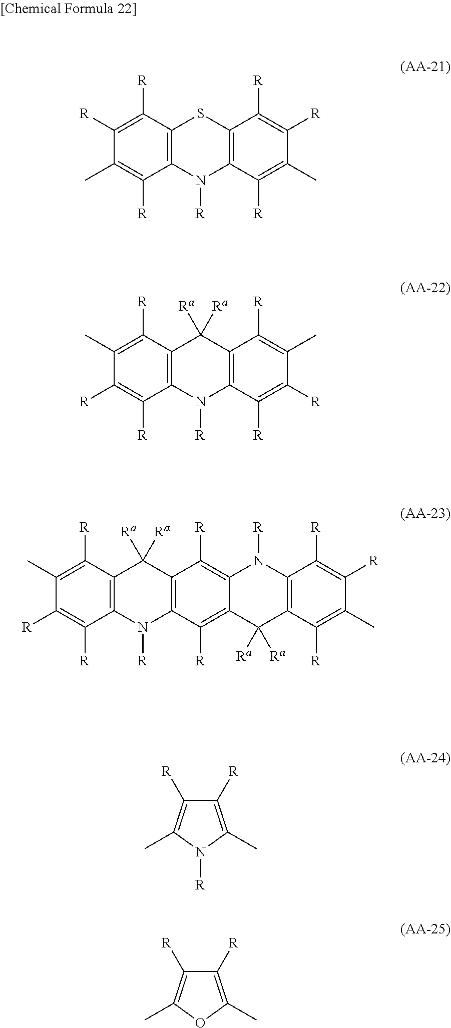

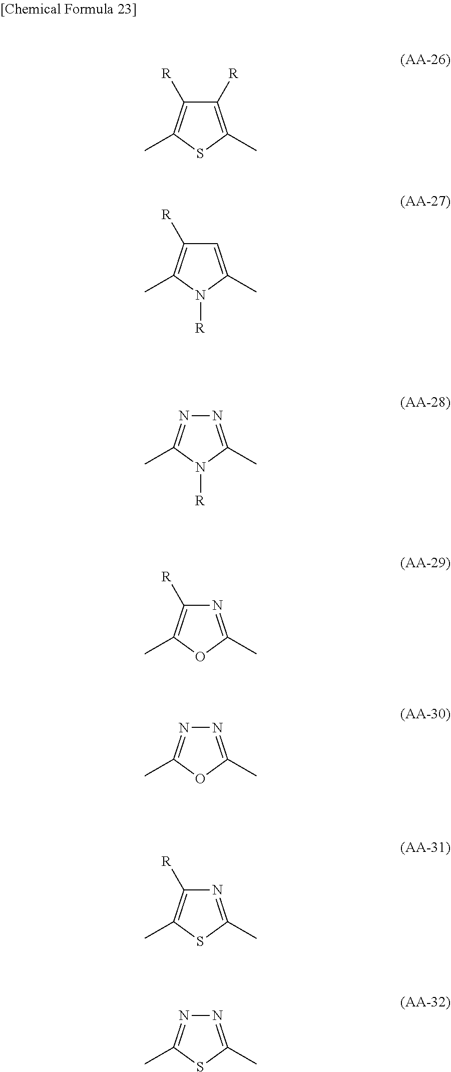

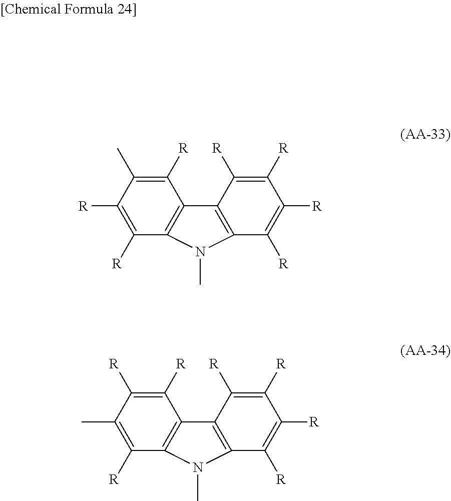

In the above-described formula (AA-1) to formula (AA-34), R and R^{a} represent the same meaning as for R and R^{a} in the above-described formula (A-1) to formula (A-20)

“The cross-linkable group” is a group capable of generating a new bond by being subjected to heating, ultraviolet ray irradiation, near ultraviolet ray irradiation, visible light irradiation, infrared ray irradiation, and/or radical reaction, and the like. The cross-linkable group is preferably a group represented by any of the following formula (B-1) to formula (B-17). These groups optionally have a substituent.

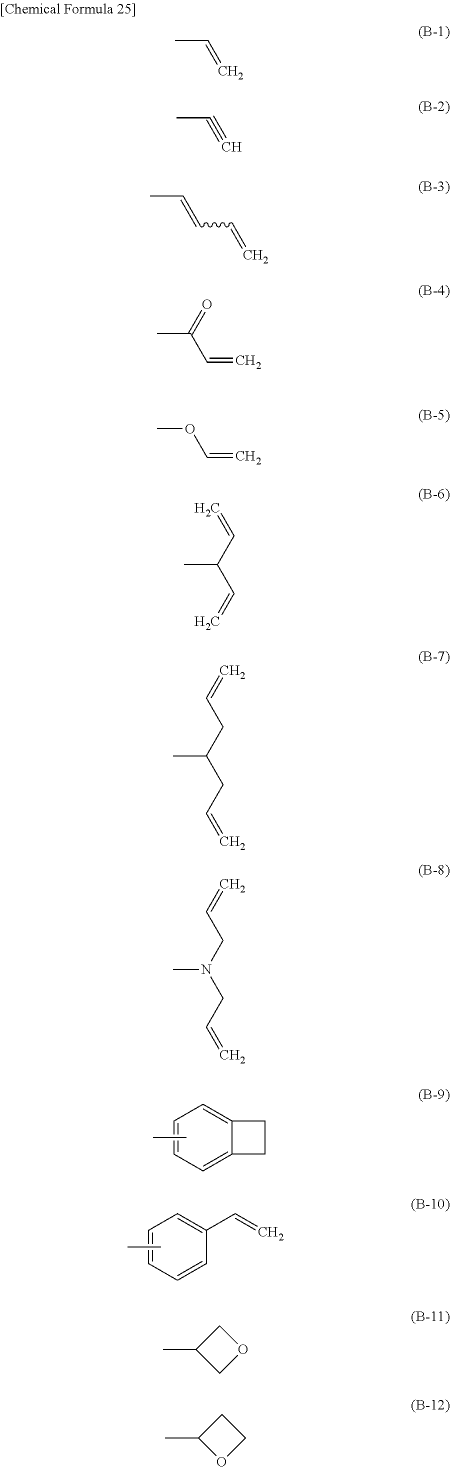

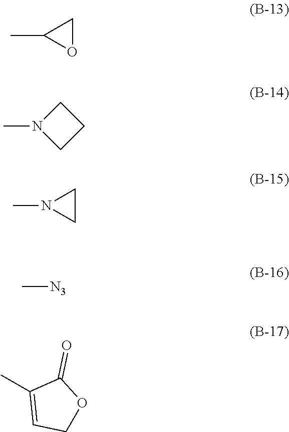

“The substituent” represents a halogen atom, a cyano group, an alkyl group, a cycloalkyl group, an aryl group, a monovalent heterocyclic ring group, an alkoxy group, a cycloalkoxy group, an aryloxy group, an amino group, a substituted amino group, an alkenyl group, a cycloalkenyl group, an alkynyl group or a cycloalkynyl group. The substituent may be a cross-linkable group.

### <Metal Complex>

The metal complex of the present embodiment is a metal complex represented by the above-described formula (0).

The ligand of which number is defined by index n¹ in the above-described formula (0) is believed to improve the electron transportability of the metal complex of the present embodiment. It is believed that the improvement in electron transportability in the metal complex causes lowering of driving voltage and enlargement of the light emission region in a light emitting device fabricated using the metal complex, resultantly improving the luminescence lifetime in the light emitting device.

The metal complex of the present embodiment is usually a metal complex showing phosphorescence at room temperature (25° C.), and preferably a metal complex showing light emission from the triplet excited state at room temperature.

The metal complex of the present embodiment is constituted of M as a central metal, a ligand of which number is defined by index n¹, and a ligand of which number is defined by index n².

It is preferable that when n¹ is 2 or 3, ligands of which number is defined by index n¹ are mutually the same.

It is preferable that when n² is 2, ligands of which number is defined by index n² are mutually the same.

M is preferably an iridium atom, since a light emitting device using the metal complex of the present embodiment shows high external quantum efficiency.

n² is preferably 0, since a light emitting device using the metal complex of the present embodiment shows high external quantum efficiency.

In Ring R^{C1}, Ring R^{C2} and Ring R^{C3}, the number of carbon atoms of the aromatic hydrocarbon ring, not including the number of carbon atoms of the substituent, is usually 6 to 60, preferably 6 to 30, and more preferably 6 to 18.

The aromatic hydrocarbon ring represented by Ring R^{C1}, Ring R^{C2} and Ring R^{C3} includes, for example, a benzene ring, a naphthalene ring, an anthracene ring, an indene ring, a fluorene ring, a spirobifluorene ring, a phenanthrene ring, a dihydrophenanthrene ring, a pyrene ring, a chrysene ring and a triphenylene ring, and is preferably a benzene ring, a naphthalene ring, an anthracene ring, a fluorene ring, a spirobifluorene ring, a phenanthrene ring or a dihydrophenanthrene ring, more preferably a benzene ring, a naphthalene ring, a fluorene ring or a spirobifluorene ring, and further preferably a benzene ring, and these rings optionally have a substituent.

In Ring R^{C1}, Ring R^{C2} and Ring R^{C3}, the number of carbon atoms of the aromatic heterocyclic ring, not including the number of carbon atoms of the substituent, is usually 2 to 60, preferably 3 to 30, and more preferably 4 to 15.

The aromatic heterocyclic ring represented by Ring R^{C1}, Ring R^{C2} and Ring R^{C3} includes, for example, a pyrrole ring, a diazole ring, a triazole ring, a furan ring, a thiophene ring, an oxadiazole ring, a thiadiazole ring, a pyridine ring, a diazabenzene ring, a triazine ring, an azanaphthalene ring, a diazanaphthalene ring, a triazanaphthalene ring, an azaanthracene ring, a diazaanthracene ring, a triazaanthracene ring, an azaphenanthrene ring, a diazaphenanthrene ring, a triazaphenanthrene ring, a dibenzofuran ring, a dibenzothiophene ring, a dibenzosilole ring, a dibenzophosphole ring, a carbazole ring, an azacarbazole ring, a diazacarbazole ring, a phenoxazine ring, a phenothiazine ring, a dihydroacridine ring and a dihydrophenazine ring, and is preferably a pyridine ring, a diazabenzene ring, an azanaphthalene ring, a diazanaphthalene ring, an azaanthracene ring, a diazaphenanthrene ring, a dibenzofuran ring, a dibenzothiophene ring, a carbazole ring, a phenoxazine ring, a phenothiazine ring, a dihydroacridine ring or a dihydrophenazine ring, more preferably a pyridine ring, a diazabenzene ring, an azanaphthalene ring, a diazanaphthalene ring, a dibenzofuran ring, a dibenzothiophene ring or a carbazole ring, and further preferably a pyridine ring or a diazabenzene ring, and these rings optionally have a substituent.

It is preferable that Ring R^{C3} is an aromatic hydrocarbon ring, and particularly a benzene ring, since synthesis thereof is easy.

Of Ring R^{C1}, Ring R^{C2} and Ring R^{C3}, at least one is preferably an aromatic hydrocarbon ring (particularly, benzene ring), two or more are more preferably aromatic hydrocarbon rings (particularly, benzene ring), and further preferably all of Ring R^{C1}, Ring R^{C2} and Ring R^{C3} are aromatic hydrocarbon rings (particularly, benzene ring).

The substituent which Ring R^{C1}, Ring R^{C2} and Ring R^{C3} optionally have is preferably an alkyl group, a cycloalkyl group, an alkoxy group, a cycloalkoxy group, an aryl group, an aryloxy group, a monovalent heterocyclic ring group, a substituted amino group or a halogen atom, more preferably an alkyl group, a cycloalkyl group, an aryl group, a monovalent heterocyclic ring group or a substituted amino group, further preferably an aryl group or a monovalent heterocyclic ring group, and particularly preferably a monovalent heterocyclic ring group. These groups optionally further have a substituent.

The number of carbon atoms of the aryl group as the substituent which Ring R^{C1}, Ring R^{C2} and Ring R^{C3} optionally have, not including the number of carbon atoms of the substituent, is usually 6 to 60, preferably 6 to 40, and more preferably 6 to 25.

Of the substituents which Ring R^{C1}, Ring R^{C2} and Ring R^{C3} optionally have, the aryl group includes, for example, groups obtained by removing from a benzene ring, a naphthalene ring, an anthracene ring, an indene ring, a fluorene ring, a spirobifluorene ring, a phenanthrene ring, a dihydrophenanthrene ring, a pyrene ring, a chrysene ring, a triphenylene ring, or a ring in which these rings are condensed one hydrogen atom bonding directly to a carbon atom constituting the ring, and is preferably a group obtained by removing from a benzene ring, a naphthalene ring, a fluorene ring, a spirobifluorene ring, a phenanthrene ring, a dihydrophenanthrene ring or a triphenylene ring one hydrogen atom bonding directly to a carbon atom constituting the ring, more preferably a group obtained by removing from a benzene ring, a fluorene ring or a spirobifluorene ring one hydrogen atom bonding directly to a carbon atom constituting the ring, and further preferably a group obtained by removing from a benzene ring one hydrogen atom bonding directly to a carbon atom constituting the ring, and these groups optionally further have a substituent.

Of the substituents which Ring R^{C1}, Ring R^{C2} and Ring R^{C3} optionally have, the aryloxy group includes groups represented by —O—Ar² (Ar² represents an aryl group, and the aryl group optionally has a substituent). The examples and the preferable range of the aryl group Ar² are the same as the examples and the preferable range of the aryl group as the substituent which Ring R^{C1}, Ring R^{C2} and Ring R^{C3} optionally have.

The number of carbon atoms of the monovalent heterocyclic ring group as the substituent which Ring R^{C1}, Ring R^{C2} and Ring R^{C3} optionally have, not including the number of carbon atoms of the substituent, is usually 2 to 60, preferably 3 to 30, and more preferably 3 to 15.

Of the substituents which Ring R^{C1}, Ring R^{C2} and Ring R^{C3} optionally have, the monovalent heterocyclic ring group includes, for example, groups obtained by removing from a pyrrole ring, a diazole ring, a triazole ring, a furan ring, a thiophene ring, an oxadiazole ring, a thiadiazole ring, a pyridine ring, a diazabenzene ring, a triazine ring, an azanaphthalene ring, a diazanaphthalene ring, a triazanaphthalene ring, an azaanthracene ring, a diazaanthracene ring, a triazaanthracene ring, an azaphenanthrene ring, a diazaphenanthrene ring, a triazaphenanthrene ring, a dibenzofuran ring, a dibenzothiophene ring, a dibenzosilole ring, a dibenzophosphole ring, a carbazole ring, an azacarbazole ring, a diazacarbazole ring, a phenoxazine ring, a phenothiazine ring, a dihydroacridine ring, a dihydrophenazine ring, or a ring in which an aromatic ring is condensed to these rings one hydrogen atom bonding directly to a carbon atom or a hetero atom constituting the ring, and is preferably a group obtained by removing from a pyridine ring, a diazabenzene ring, a triazine ring, an azanaphthalene ring, a diazanaphthalene ring, a dibenzofuran ring, a dibenzothiophene ring, a carbazole ring, an azacarbazole ring, a diazacarbazole ring, a phenoxazine ring, a phenothiazine ring, a dihydroacridine ring or a dihydrophenazine ring one hydrogen atom bonding directly to a carbon atom or a hetero atom constituting the ring, more preferably a group obtained by removing from a pyridine ring, a diazabenzene ring, a triazine ring, a dibenzofuran ring, a dibenzothiophene ring, a carbazole ring, an azacarbazole ring, a diazacarbazole ring, a phenoxazine ring, a phenothiazine ring, a dihydroacridine ring or a dihydrophenazine ring one hydrogen atom bonding directly to a carbon atom or a hetero atom constituting the ring, further preferably a group obtained by removing from a dibenzofuran ring, a dibenzothiophene ring, a carbazole ring, a phenoxazine ring, a phenothiazine ring, a dihydroacridine ring or a dihydrophenazine ring one hydrogen atom bonding directly to a carbon atom or a hetero atom constituting the ring, and particularly preferably a group obtained by removing from a dibenzofuran ring or a dibenzothiophene ring one hydrogen atom bonding directly to a carbon atom constituting the ring, and these rings optionally have a substituent.

In the substituted amino group as the substituent which Ring R^{C1}, Ring R^{C2} and Ring R^{C3} optionally have, the substituent which an amino group has is preferably an aryl group or a monovalent heterocyclic ring group, and more preferably an aryl group, and these groups optionally further have a substituent. The examples and the preferable range of the aryl group as the substituent which an amino group has are the same as the examples and the preferable range of the aryl group as the substituent which Ring R^{C1}, Ring R^{C2} and Ring R^{C3} optionally have. The examples and the preferable range of the monovalent heterocyclic ring group as the substituent which an amino group has are the same as the examples and the preferable range of the monovalent heterocyclic ring group as the substituent which Ring R^{C1}, Ring R^{C2} and Ring R^{C3} optionally have.

Of the substituents which Ring R^{C1}, Ring R^{C2} and Ring R^{C3} optionally have, the halogen atom is preferably a fluorine atom or a chlorine atom, and more preferably a fluorine atom.

The substituent which the substituent which Ring R^{C1}, Ring R^{C2} and Ring R^{C3} optionally have optionally further has is preferably an alkyl group, a cycloalkyl group, an alkoxy group, a cycloalkoxy group, an aryl group, an aryloxy group, a monovalent heterocyclic ring group, a substituted amino group or a halogen atom, more preferably an alkyl group, a cycloalkyl group, an aryl group or a monovalent heterocyclic ring group, further preferably an alkyl group or an aryl group, and particularly preferably an aryl group, and these groups optionally further have a substituent (for example, an alkyl group).

The aryl group as the substituent which the substituent which Ring R^{C1}, Ring R^{C2} and Ring R^{C3} optionally have optionally further has is preferably a phenyl group, and this phenyl group optionally further has a substituent (for example, an alkyl group).

The alkyl group as the substituent which the substituent which Ring R^{C1}, Ring R^{C2} and Ring R^{C3} optionally have optionally further has is preferably an alkyl group having 3 to 9 carbons.

The examples and the preferable ranges of the aryl group, the aryloxy group, the monovalent heterocyclic ring group, the substituted amino group and the halogen atom as the substituent which the substituent which Ring R^{C1}, Ring R^{C2} and Ring R^{C3} optionally have optionally further has are the same as the examples and the preferable ranges of the aryl group, the aryloxy group, the monovalent heterocyclic ring group, the substituted amino group and the halogen atom as the substituent which Ring R^{C1}, Ring R^{C2} and Ring R^{C3} optionally have, respectively.

It is preferable that at least one of Ring R^{C2} and Ring R^{C3} (particularly, Ring R^{C2}) has an aryl group or a monovalent heterocyclic ring group as a substituent, and/or at least one of R³, R⁴ and R⁵ (particularly, R⁴ or R⁵) is an aryl group or a monovalent heterocyclic ring group, since the long term deterioration of a light emitting device containing the metal complex of the present embodiment is more suppressed. Further, it is particularly preferable that at least one of Ring R^{C2} and Ring R^{C3} (particularly, Ring R^{C2}) has an aryl group or a monovalent heterocyclic ring group as a substituent, and, at least one of R³, R⁴ and R⁵ (particularly, R⁴ or R⁵) is an aryl group or a monovalent heterocyclic ring group.

When at least one of Ring R^{C1}, Ring R^{C2} and Ring R^{C3} (particularly, Ring R^{C2}) has an aryl group or a monovalent heterocyclic ring group as a substituent, and/or at least one of R³, R⁴ and R⁵ (particularly, R⁴ or R⁵) is an aryl group or a monovalent heterocyclic ring group, it is particularly preferable that the aryl group is a phenyl group, and this group optionally further has a substituent.

When at least one of Ring R^{C1}, Ring R^{C2} and Ring R^{C3} (particularly, Ring R^{C2}) has an aryl group or a monovalent heterocyclic ring group as a substituent, and/or at least one of R³, R⁴ and R⁵ (particularly, R⁴ or R⁵) is an aryl group or a monovalent heterocyclic ring group, it is particularly preferable that the monovalent heterocyclic ring group is a group obtained by removing from a pyrimidine ring or a triazine ring (particularly, triazine ring) one hydrogen atom bonding directly to a carbon atom constituting the ring, and these groups optionally further have a substituent.

At least one of Ring R^{C1}, Ring R^{C2} and Ring R^{C3} preferably has an aryl group or a monovalent heterocyclic ring group as a substituent, at least one of Ring R^{C1}, Ring R^{C2} and Ring R^{C3} more preferably has an aryl group as a substituent, and Ring R^{C2} further preferably has an aryl group as a substituent, since the long term deterioration of a light emitting device containing the metal complex of the present embodiment is more suppressed.

The aryl group as the substituent of Ring R^{C1}, Ring R^{C2} and Ring R^{C3} is particularly preferably a phenyl group, and this phenyl group optionally further has a substituent.

The monovalent heterocyclic ring group as the substituent of Ring R^{C1}, Ring R^{C2} and Ring R^{C3} is particularly preferably a group obtained by removing from a pyrimidine ring or a triazine ring (particularly, a triazine ring) one hydrogen atom bonding directly to a carbon atom constituting the ring, and these groups optionally further have a substituent.

Ring R^{C1}, Ring R^{C2} or Ring R^{C3} optionally has a group represented by the above-described formula (D-A), the above-described formula (D-B) or the above-described formula (D-C) as a substituent. When Ring R^{C1}, Ring R^{C2} or Ring R^{C3} has a group represented by the above-described formula (D-A), the above-described formula (D-B) or the above-described formula (D-C) as a substituent, aggregation of a metal complex is suppressed, leading to further improvement in the luminescence lifetime of a light emitting device formed using the metal complex in some cases.

Ring R^{C3} includes, for example, a group represented by the above-described formula (P′).

The examples and the preferable ranges of the aryl group, the aryloxy group, the monovalent heterocyclic ring group, the substituted amino group and the halogen atom as R^{C31}, R^{C32}, R^{C33} and R^{C34} are the same as the examples and the preferable ranges of the aryl group, the aryloxy group, the monovalent heterocyclic ring group, the substituted amino group and the halogen atom as the substituent which Ring R^{C1}, Ring R^{C2} and Ring R^{C3} optionally have, respectively.

The examples and the preferable range of the substituent which R^{C31}, R^{C32}, R^{C33} and R^{C34} optionally further have are the same as the examples and the preferable range of the substituent which the substituent which Ring R^{C1}, Ring R^{C2} and Ring R^{C3} optionally have optionally further has.

R^{C31}, R^{C32}, R^{C33} and R^{C34} represent preferably a hydrogen atom, an alkyl group, a cycloalkyl group, an aryl group or a monovalent heterocyclic ring group, more preferably a hydrogen atom, an alkyl group, a cycloalkyl group or an aryl group, and further preferably a hydrogen atom, an alkyl group or an aryl group, since synthesis of the metal complex of the present embodiment is easy.

R^{C31}, R^{C32}, R^{C33} or R^{C34} may be a group represented by the above-described formula (D-A), the above-described formula (D-B) or the above-described formula (D-C). When R^{C31}, R^{C32}, R^{C33} or R^{C34} is a group represented by the above-described formula (D-A), the above-described formula (D-B) or the above-described formula (D-C), aggregation of a metal complex is suppressed, leading to further improvement in the luminescence lifetime of a light emitting device formed using the metal complex in some cases.

As the metal complex of the present embodiment, a metal complex in which one combination of R² and R³, R³ and R⁴, and R⁵ and R⁶ is integrated to form a group represented by the above-described formula (P) is more preferred, a metal complex in which one combination of R² and R³, and R³ and R⁴ is integrated to form a group represented by the above-described formula (P) is further preferred, and a metal complex in which a combination of R² and R³ is integrated to form a group represented by the above-described formula (P) is particularly preferred, since synthesis is easy. Of the metal complexes represented by the above-described formula (0), the metal complex in which a combination of R² and R³ is integrated to form a group represented by the above-described formula (P) is hereinafter referred to as “metal complex X”.

The metal complex X includes, for example, metal complexes represented by the above-described formula (2-A1), the above-described formula (2-B1) or the above-described formula (2-C1), and it is more preferably a metal complex represented by the above-described formula (2-A1) or the above-described formula (2-B1), and further preferably a metal complex represented by the above-described formula (2-A1), since synthesis is easy.

E¹, E², E³, E⁴, E⁵ and E⁶ represent preferably a carbon atom, since synthesis of the metal complex of the present embodiment is easy.

Regarding X^{a} and X^{b}, it is preferable that X^{a} is a group represented by —C(R^{Xa})₂— or a group represented by —C(R^{Xa})₂—C(R^{Xa})₂—, and X^{b} is a group represented by a single bond, and it is further preferable that X^{a} is a group represented by —C(R^{Xa})₂—, and X^{b} is a group represented by a single bond, since synthesis of the metal complex of the present embodiment is easy.

When X^{a} or X^{b} is a group represented by —C(R^{Xa})₂—, it is preferable that two groups R^{Xa} each independently represent an alkyl group (particularly, an alkyl group having 1 to 8 carbons) or an aryl group (particularly, a phenyl group), or it is preferable that —C(R^{Xa})₂— is a group represented by the following formula (XAB-1).

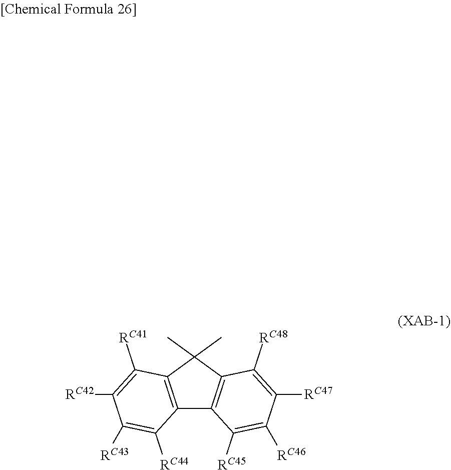

[wherein, R^{C41}, R^{C42}, R^{C43}, R^{C44}, R^{C45}, R^{C46}, R^{C47} and R^{C48} each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent].

In the above-described formula (XAB-1), R^{C41}, R^{C42}, R^{C43}, R^{C44}, R^{C45}, R^{C46}, R^{C47} and R^{C48} each independently represent preferably a hydrogen atom or an alkyl group having 1 to 8 carbons.

Regarding Y^{a} and Y^{b}, it is preferable that Y^{a} is a single bond, and Y^{b} is a group represented by —C(R^{Ya})₂—, since synthesis of the metal complex of the present embodiment is easy.

When Y^{a} or Y^{b} is a group represented by —C(R^{Ya})₂—, it is preferable that two groups R^{Ya} each independently represent an alkyl group (particularly, an alkyl group having 1 to 8 carbons) or an aryl group (particularly, a phenyl group) (more preferably, an alkyl group), or it is preferable that —C(R^{Ya})₂— is a group represented by the above-described formula (XAB-1).

The examples and the preferable ranges of the aryl group, the aryloxy group, the monovalent heterocyclic ring group, the substituted amino group and the halogen atom as the R¹, R², R³, R⁴, R⁵ and R⁶ are the same as the examples and the preferable ranges of the aryl group, the aryloxy group, the monovalent heterocyclic ring group, the substituted amino group and the halogen atom as the substituent which Ring R^{C1}, Ring R^{C2} and Ring R^{C3} optionally have, respectively

The examples and the preferable range of the substituent which R¹, R², R³, R⁴, R⁵ and R⁶ optionally further have are the same as the examples and the preferable range of the substituent which the substituent which Ring R^{C1}, Ring R^{C2} and Ring R^{C3} optionally have optionally further has.

R¹, R², R³, R⁴, R⁵ and R⁶ represent preferably a hydrogen atom, an alkyl group, a cycloalkyl group, an aryl group, a monovalent heterocyclic ring group or a halogen atom, more preferably a hydrogen atom, an alkyl group, a cycloalkyl group or an aryl group, and further preferably a hydrogen atom, since synthesis of the metal complex of the present embodiment is easy.

At least one of R¹, R², R³, R⁴, R⁵ or R⁶ (particularly, R⁴ or R⁵) is preferably an aryl group or a monovalent heterocyclic ring group, since the long term deterioration of a light emitting device containing the metal complex of the present embodiment is more suppressed.

When at least one of R¹, R², R³, R⁴, R⁵ or R⁶ (particularly, R⁴ or R⁵) is an aryl group, the aryl group is particularly preferably a phenyl group, and this phenyl group optionally further has a substituent.

When at least one of R¹, R², R³, R⁴, R⁵ or R⁶ (particularly, R⁴ or R⁵) is a monovalent heterocyclic ring group, the monovalent heterocyclic ring group is particularly preferably a group obtained by removing from a triazine ring one hydrogen atom bonding directly to a carbon atom constituting the ring, and further optionally has a substituent.

R¹, R², R³, R⁴, R⁵ or R⁶ (particularly, R³ or R⁵) may be a group represented by the above-described formula (D-A), the above-described formula (D-B) or the above-described formula (D-C). When R¹, R², R³, R⁴, R⁵ or R⁶ (particularly, R³ or R⁵) has a group represented by the above-described formula (D-A), the above-described formula (D-B) or the above-described formula (D-C), aggregation of a metal complex is suppressed, leading to further improvement in the luminescence lifetime of a light emitting device formed using the metal complex in some cases.

In particular, R³, R⁴ or R⁵ (further, R⁴ or R⁵) may be a group represented by the above-described formula (D-A), the above-described formula (D-B) or the above-described formula (D-C). When R³, R⁴ or R⁵ (further, R⁴ or R⁵) has a group represented by the above-described formula (D-A), the above-described formula (D-B) or the above-described formula (D-C), aggregation of a metal complex is suppressed, leading to further improvement in the luminescence lifetime of a light emitting device formed using the metal complex in some cases.

The metal complex represented by the above-described formula (2-A1) includes, for example, metal complexes represented by the above-described formula (2-A1-1).

The examples and the preferable ranges of the aryl group, the aryloxy group, the monovalent heterocyclic ring group, the substituted amino group and the halogen atom as R^{C41}, R^{C42}, R^{C43}, R^{C44}, R^{C45}, R^{C46}, R^{C47} and R^{C48} are the same as the examples and the preferable ranges of the aryl group, the aryloxy group, the monovalent heterocyclic ring group, the substituted amino group and the halogen atom as the substituent which Ring R^{C1}, Ring R^{C2} and Ring R^{C3} optionally have, respectively.

The examples and the preferable range of the substituent which R^{C41}, R^{C42}, R^{C43}, R^{C44}, R^{C45}, R^{C46}, R^{C47} and R^{C48} optionally further have are the same as the examples and the preferable range of the substituent which the substituent which Ring R^{C1}, Ring R^{C2} and Ring R^{C3} optionally have optionally further has.

R^{C41}, R^{C42}, R^{C43}, R^{C44}, R^{C45}, R^{C46}, R^{C47} and R^{C48} represent preferably a hydrogen atom, an alkyl group, a cycloalkyl group, an aryl group, a monovalent heterocyclic ring group or a halogen atom, more preferably a hydrogen atom, an alkyl group, a cycloalkyl group or an aryl group, and further preferably a hydrogen atom, since synthesis of the metal complex of the present embodiment is easy.

R^{C41}, R^{C42}, R^{C43}, R^{C44}, R^{C45}, R^{C46}, R^{C47} or R^{C48} may be a group represented by the above-described formula (D-A), the above-described formula (D-B) or the above-described formula (D-C). When R^{C41}, R^{C42}, R^{C43}, R^{C44}, R^{C45}, R^{C46}, R^{C47} or R^{C48} has a group represented by the above-described formula (D-A), the above-described formula (D-B) or the above-described formula (D-C), aggregation of a metal complex is suppressed, leading to further improvement in the luminescence lifetime of a light emitting device formed using the metal complex in some cases.

<Group Represented by the Formula (D-A) to Formula (D-C)>

m^{DA1}, m^{DA2}, m^{DA3}, m^{DA4}, m^{DA5}, m^{DA6} and m^{DA7} represent usually an integer of 10 or less, preferably an integer of 5 or less, more preferably an integer of 2 or less, and further preferably 0 or 1. It is preferable that m^{DA2}, m^{DA3}, m^{DA4}, m^{DA5}, m^{DA6} and m^{DA7} are the same integer.

G^{DA} is preferably an aromatic hydrocarbon group or a heterocyclic ring group, and more preferably a group obtained by removing from a benzene ring, a pyridine ring, a pyrimidine ring, a triazine ring or a carbazole ring three hydrogen atoms bonding directly to carbon atoms or nitrogen atoms constituting the ring, and these groups optionally have a substituent.

The substituent which G^{DA} optionally has is preferably an alkyl group, a cycloalkyl group, an alkoxy group, a cycloalkoxy group, an aryl group or a monovalent heterocyclic ring group, and these groups optionally have a substituent.

G^{DA} is preferably a group represented by the following formula (GDA-11) to formula (GDA-15).

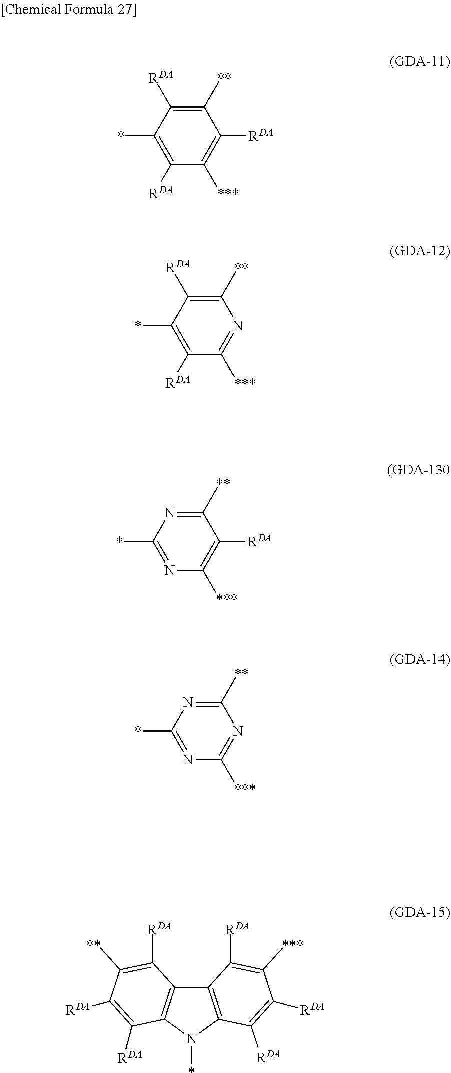

[wherein,

* represents a bond to Ar^{DA1} in the formula (D-A), Ar^{DA1} in the formula (D-B), Ar^{DA2} in the formula (D-B), or Ar^{DA3} in the formula (D-B);

** represents a bond to Ar^{DA2} in the formula (D-A), Ar^{DA2} in the formula (D-B), Ar^{DA4} in the formula (D-B), or Ar^{DA6} in the formula (D-B);

*** represents a bond to Ar^{DA3} in the formula (D-A), Ar^{DA3} in the formula (D-B), Ar^{DA5} in the formula (D-B), or Ar^{DA7} in the formula (D-B);

R^{DA} represents a hydrogen atom, an alkyl group, a cycloalkyl group, an alkoxy group, a cycloalkoxy group, an aryl group or a monovalent heterocyclic ring group, and these groups optionally further have a substituent; when a plurality of R^{DA} are present, they may be the same or different].

Ar^{DA1}, Ar^{DA2}, Ar^{DA3}, Ar^{DA4}, Ar^{DA5}, Ar^{DA6} and Ar^{DA7} represent preferably a phenylene group, a fluorenediyl group or a carbazolediyl group, and more preferably a group represented by the following formula (ArDA-1) to formula (ArDA-5), and these groups optionally have a substituent.

The examples and the preferable range of the substituent which Ar^{DA1}, Ar^{DA2}, Ar^{DA3}, Ar^{DA4}, Ar^{DA5}, Ar^{DA6} and Ar^{DA7} optionally have are the same as the examples and the preferable range of the substituent which G^{DA} optionally has.

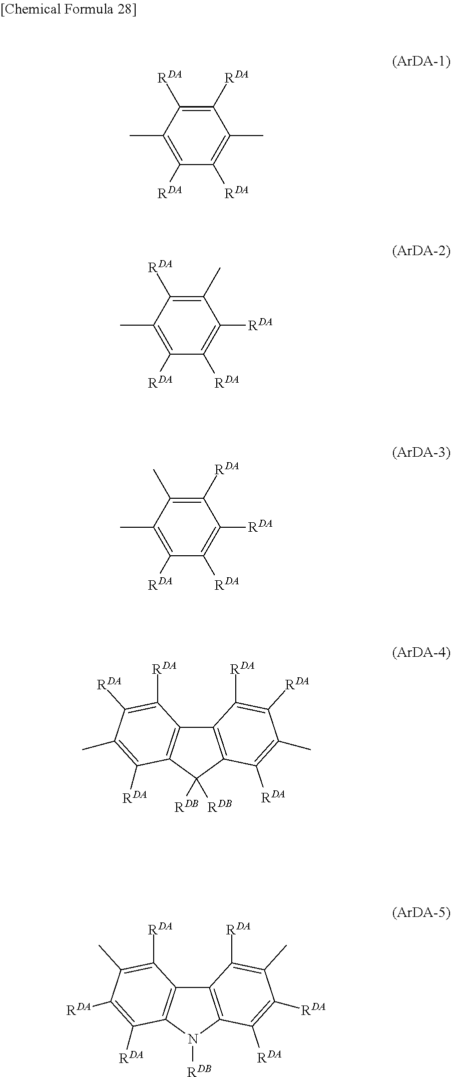

[wherein,

R^{DA} represents the same meaning as for R^{DA} in the above-described formula (GDA-11) to formula (GDA-15);

R^{DB} represents a hydrogen atom, an alkyl group, a cycloalkyl group, an aryl group or a monovalent heterocyclic ring group, and these groups optionally have a substituent; when a plurality of R^{DB} are present, they may be the same or different].

The substituent which T^{DA} optionally has is preferably an alkyl group, a cycloalkyl group, an aryl group or a monovalent heterocyclic ring group, more preferably an alkyl group, a cycloalkyl group or an aryl group, further preferably an alkyl group or an aryl group, and particularly preferably an alkyl group, and these groups optionally further have a substituent.

T^{DA} is preferably a group represented by the following formula (TDA-1) to formula (TDA-3), and more preferably a group represented by the following formula (TDA-1).

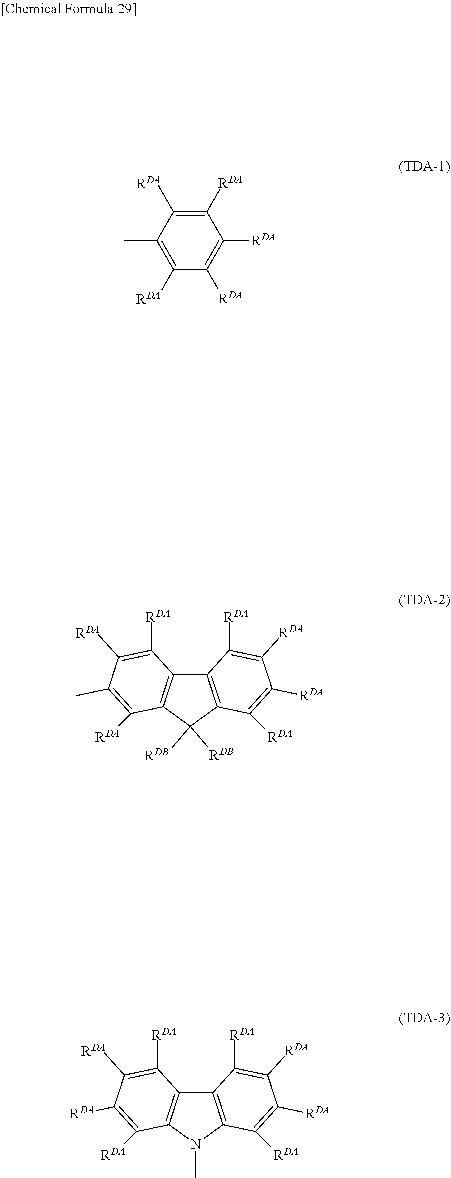

[wherein, R^{DA} and R^{DB} represent the same meaning as for R^{DA} and R^{DB} in the above-described formula (ArDA-1) to formula (ArDA-5).].

The group represented by the above-described formula (D-A) includes, for example, groups represented by the following formula (D-A-1) to formula (D-A-12).

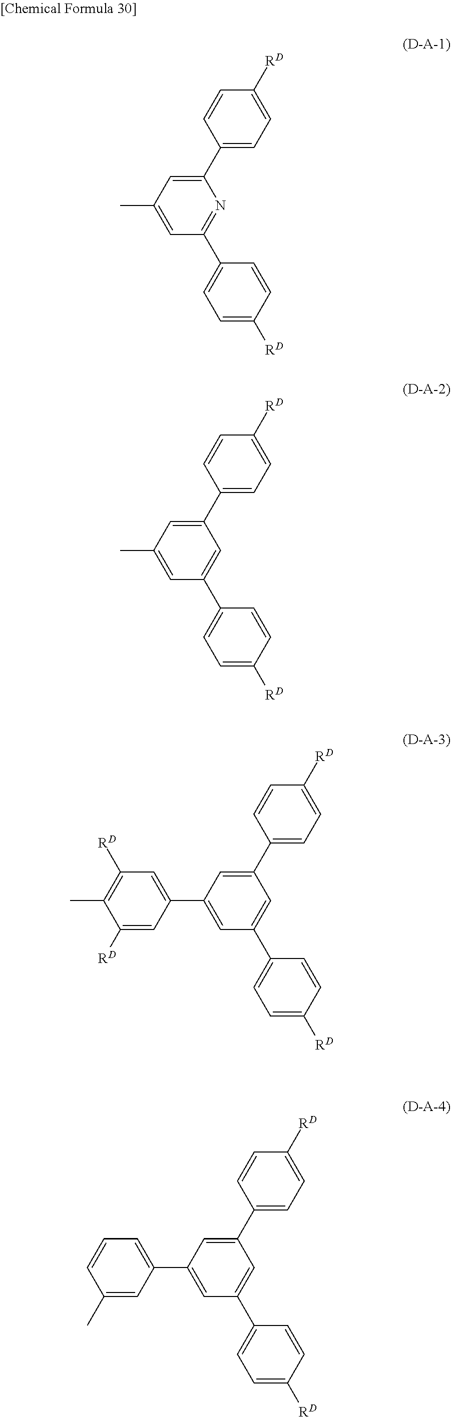

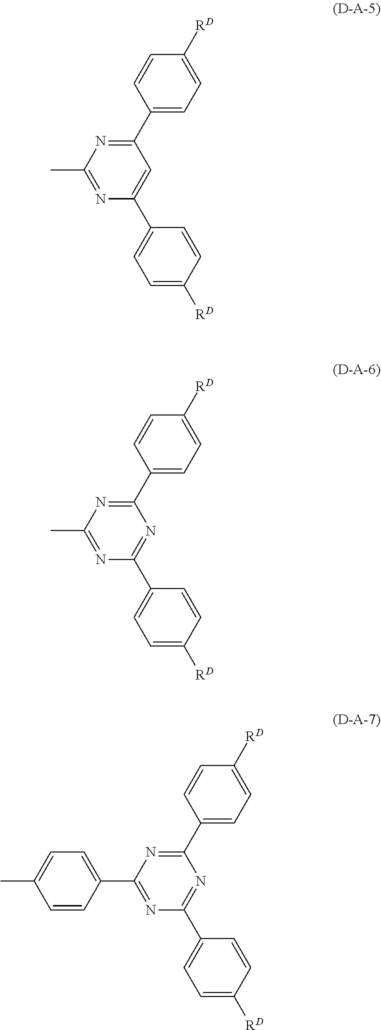

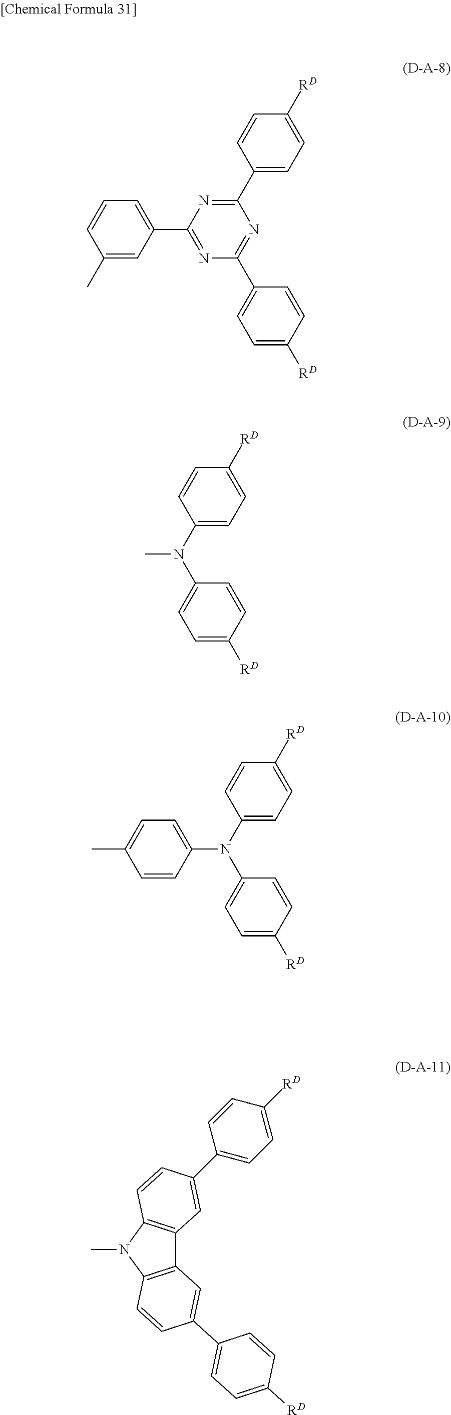

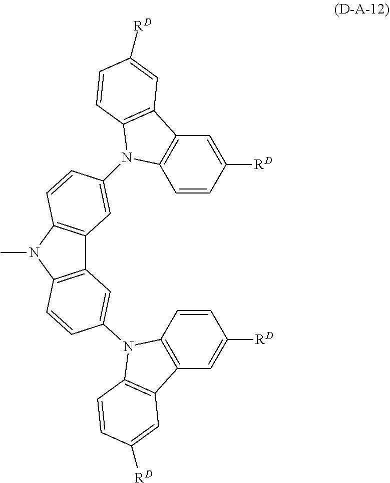

[wherein, R^{D} represents a hydrogen atom, an alkyl group, a cycloalkyl group, an alkoxy group or a cycloalkoxy group, and these groups optionally have a substituent; when a plurality of R^{D} are present, they may be the same or different].

The group represented by the above-described formula (D-B) includes, for example, groups represented by the following formula (D-B-1) to formula (D-B-7).

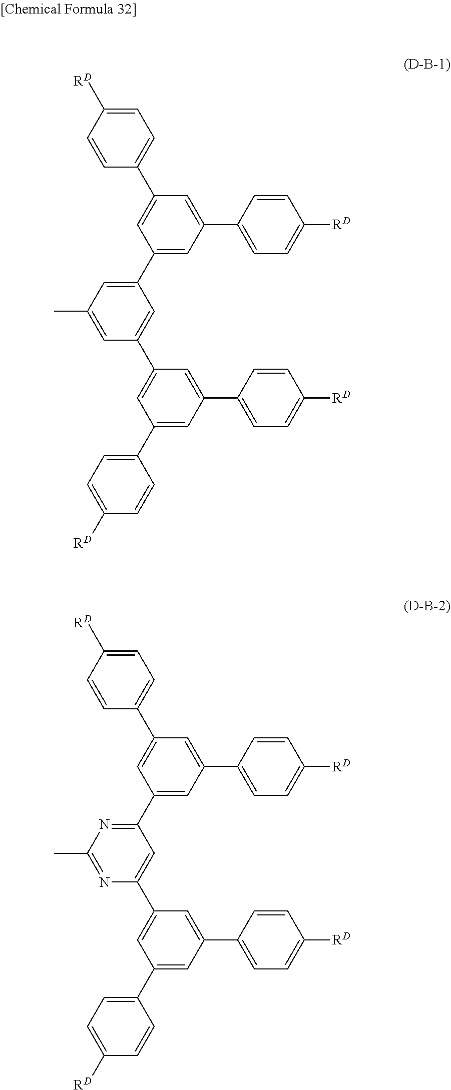

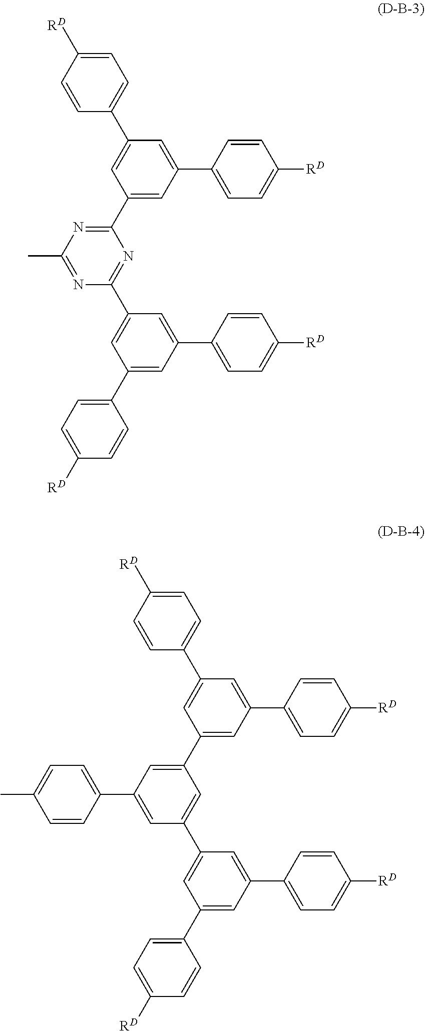

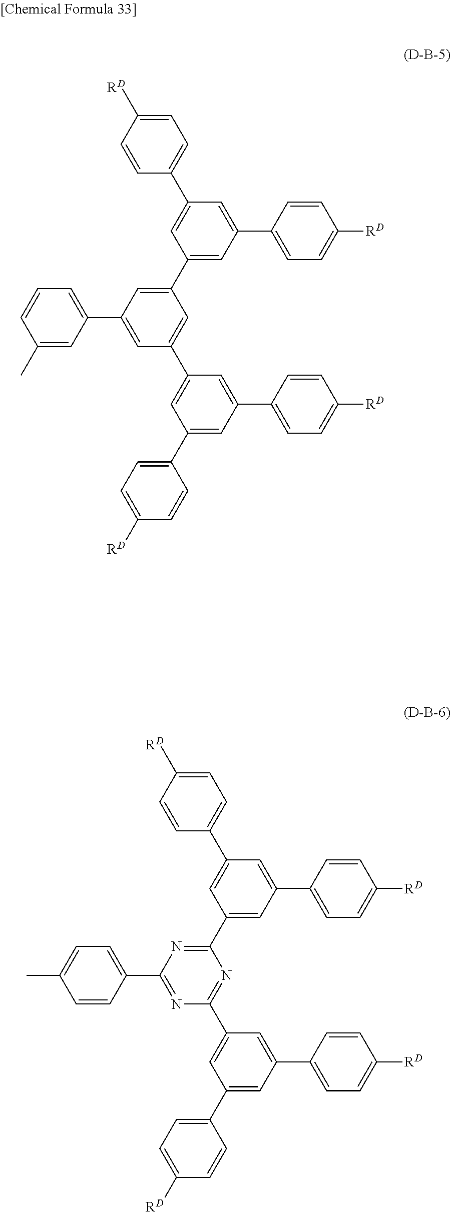

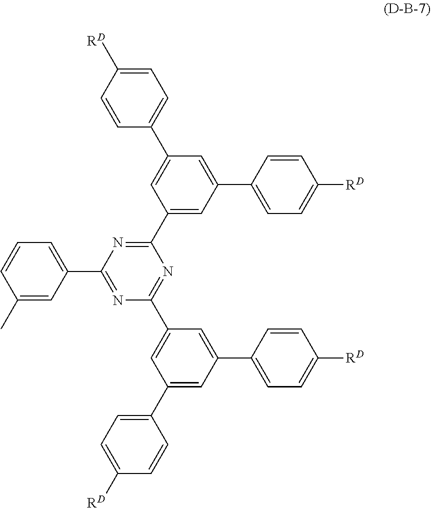

[wherein, R^{D} represents the same meaning as for R^{D} in the above-described formula (D-A-1) to formula (D-A-12)].

The group represented by the above-described formula (D-C) includes, for example, groups represented by the following formula (D-C-1) to formula (D-C-14).

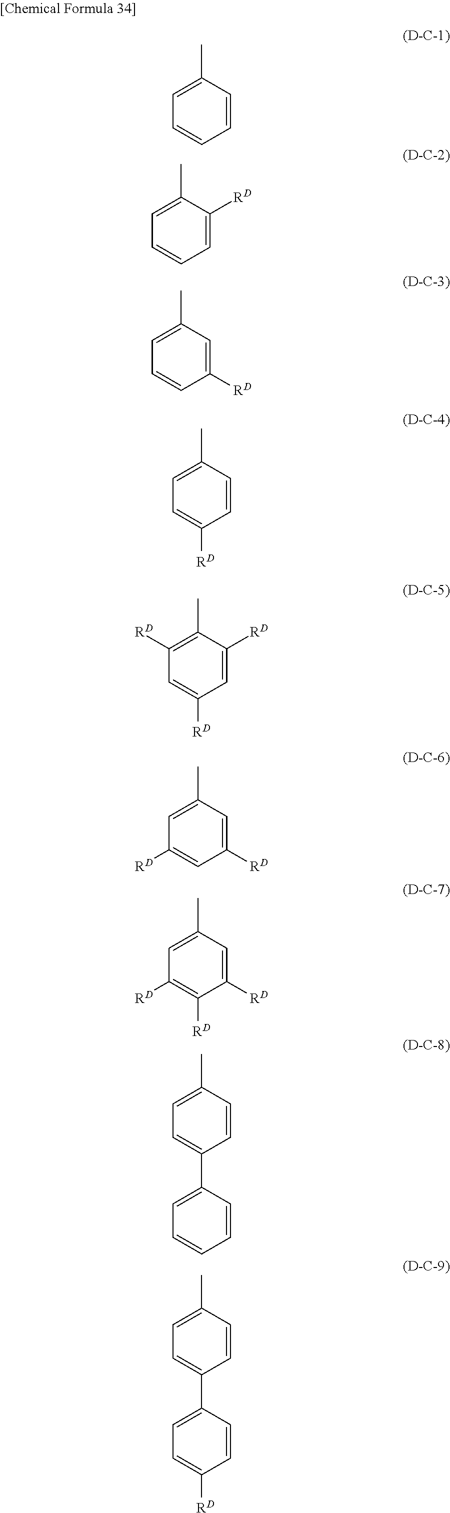

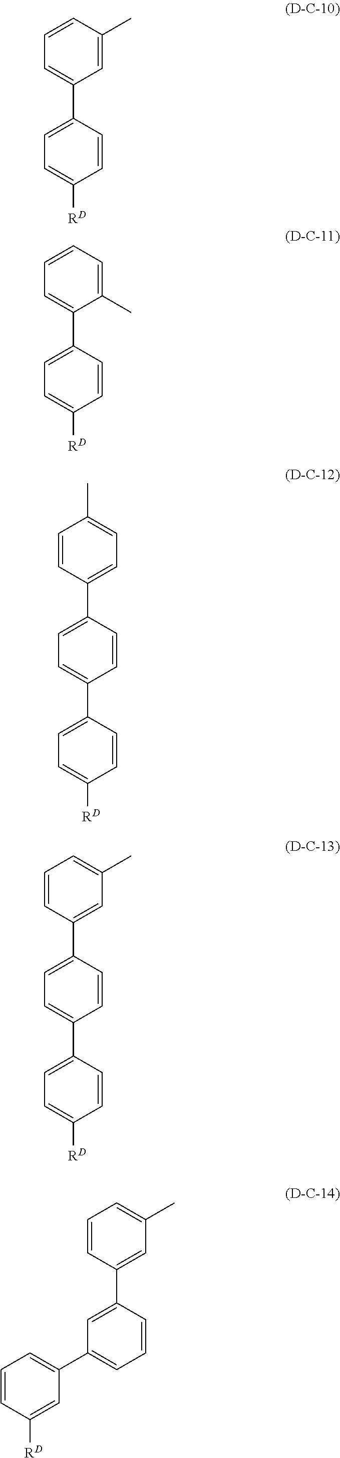

[wherein, R^{D} represents the same meaning as for R^{D} in the above-described formula (D-A-1) to formula (D-A-12)].

In one embodiment according to the present disclosure, the metal complex has a structure in which a group represented by the above-described formula (D-A), formula (D-B) or formula (D-C) is bonded to Ring R^{C1}, Ring R^{C2} or Ring R^{C3} in the above-described formula (0) When the metal complex has a structure in which a group represented by the above-described formula (D-A), formula (D-B) or formula (D-C) is bonded to Ring R^{C1}, Ring R^{C2} or Ring R^{C3} in the above-described formula (0), aggregation of a metal complex is suppressed, leading to further improvement in the luminescence lifetime of a light emitting device formed using the metal complex in some cases.

In another embodiment according to the present disclosure, the metal complex has a structure in which a group represented by the above-described formula (D-A), formula (D-B) or formula (D-C) is bonded to Ring R^{C2} or Ring R^{C3} in the above-described formula (0), or a structure in which R³ or R⁵ in the above-described formula (0) is a group represented by the above-described formula (D-A), formula (D-B) or formula (D-C). When the metal complex has a structure in which a group represented by the above-described formula (D-A), formula (D-B) or formula (D-C) is bonded to Ring R^{C2} or Ring R^{C3} in the above-described formula (0), or a structure in which R³ or R⁵ in the above-described formula (0) is a group represented by the above-described formula (D-A), formula (D-B) or formula (D-C), aggregation of a metal complex is suppressed, leading to further improvement in the luminescence lifetime of a light emitting device formed using the metal complex in some cases.

### <Anionic Bidentate Ligand>

The anionic bidentate ligand represented by A¹-G¹-A² includes, for example, ligands represented by the following formulae. However, the anionic bidentate ligand represented by A¹-G¹-A² is different from a ligand of which number is defined by index n¹.

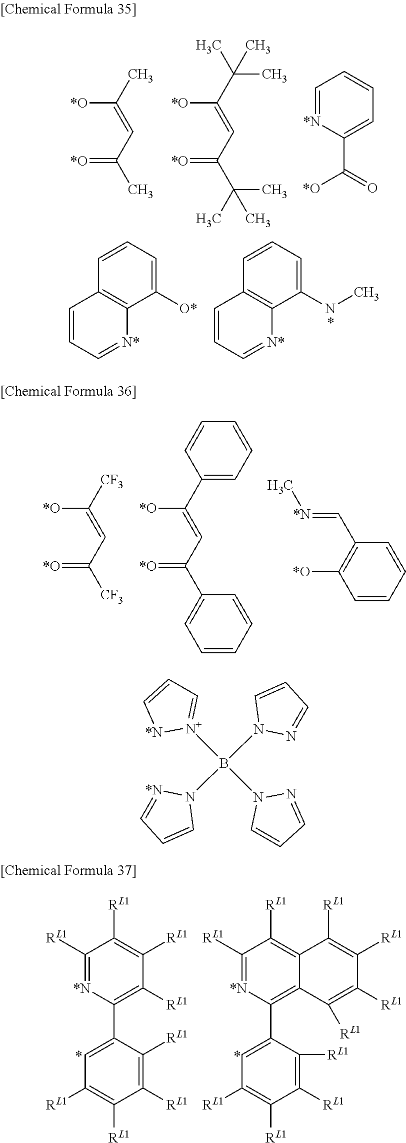

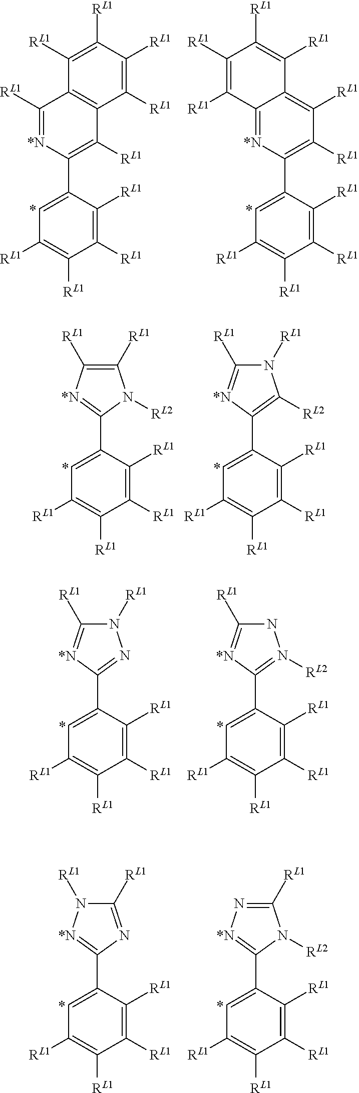

[wherein,

* represents a site binding to M;

R^{L1} represents a hydrogen atom, an alkyl group, a cycloalkyl group, an aryl group, a monovalent heterocyclic ring group or a halogen atom, and these groups optionally have a substituent; a plurality of R^{L1} may be the same or different;

R^{L2} represents an alkyl group, a cycloalkyl group, an aryl group, a monovalent heterocyclic ring group or a halogen atom, and these groups optionally have a substituent].

Specific examples of the metal complex of the present embodiment include metal complexes represented by the following formula (Ir-101) to formula (Ir-136).

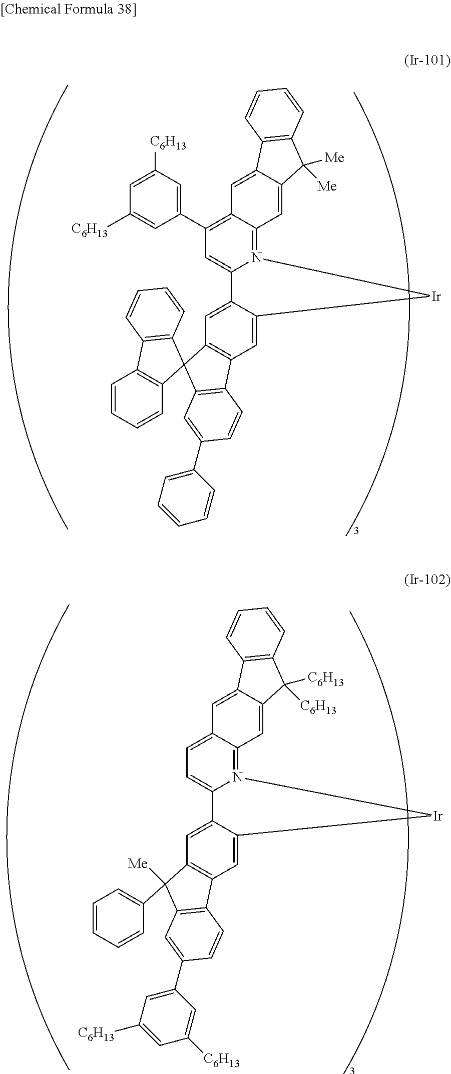

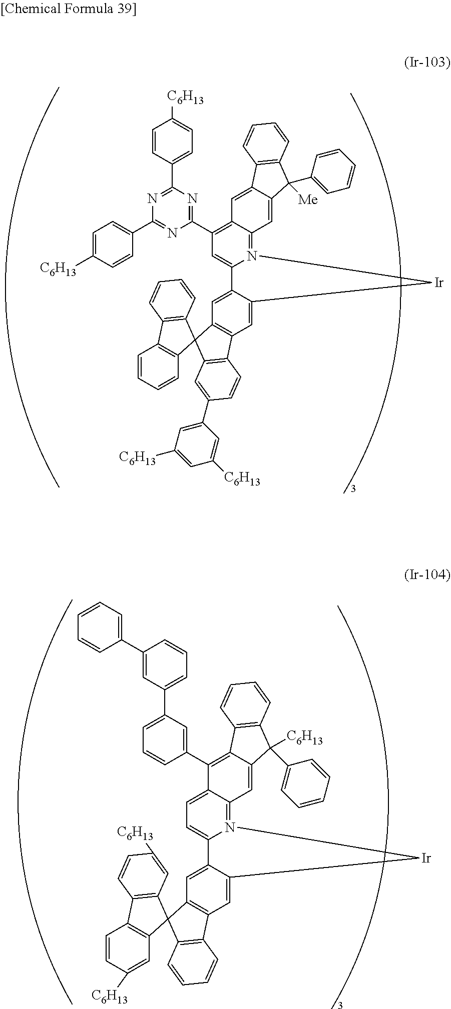

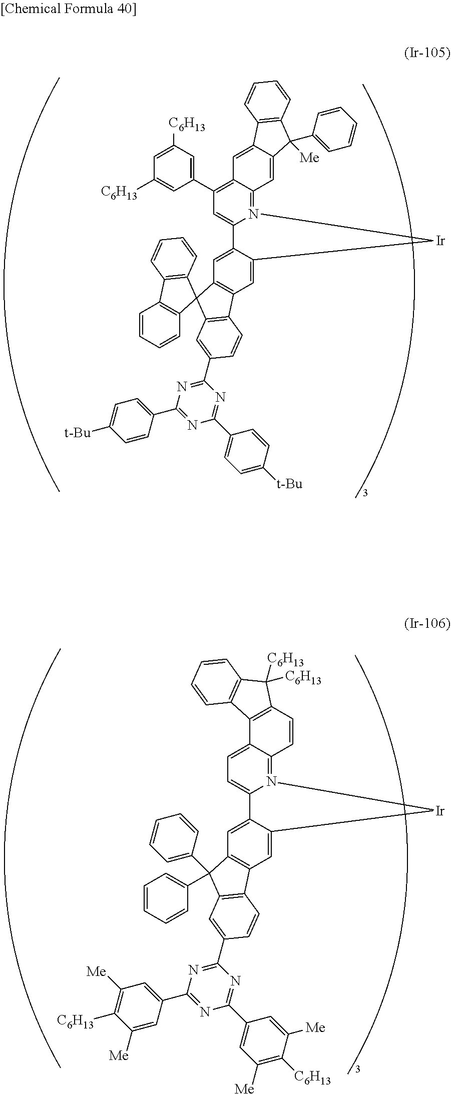

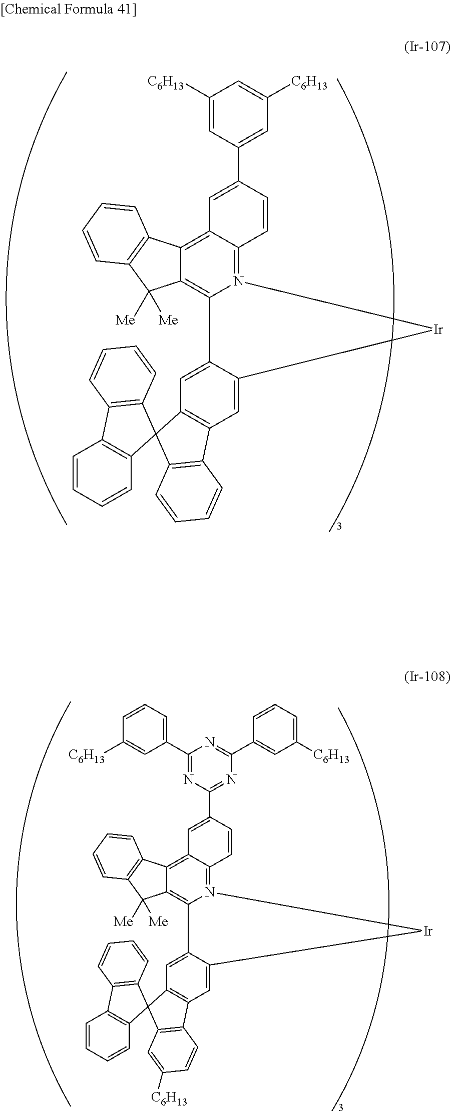

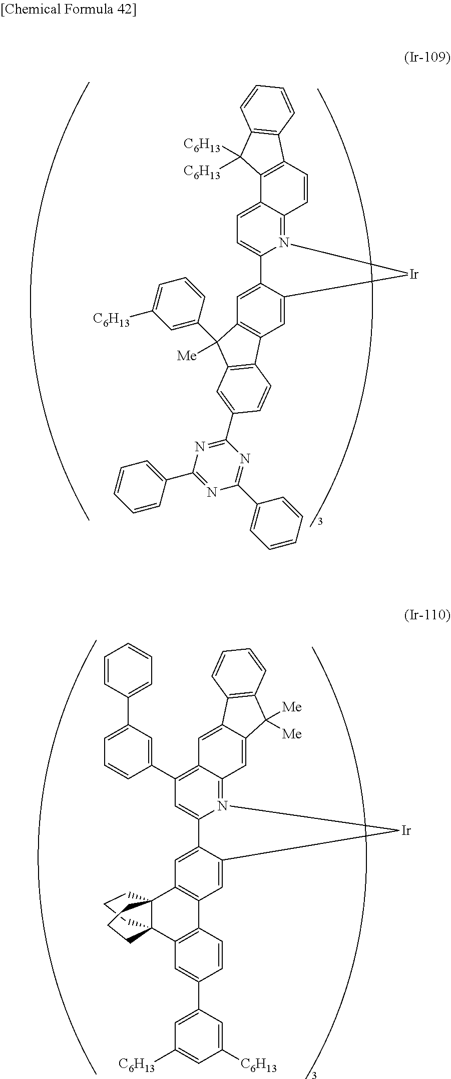

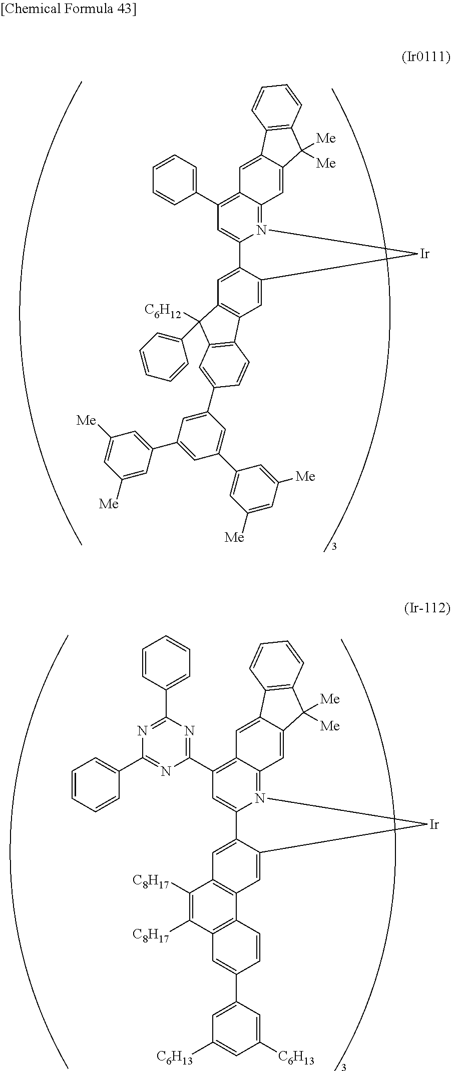

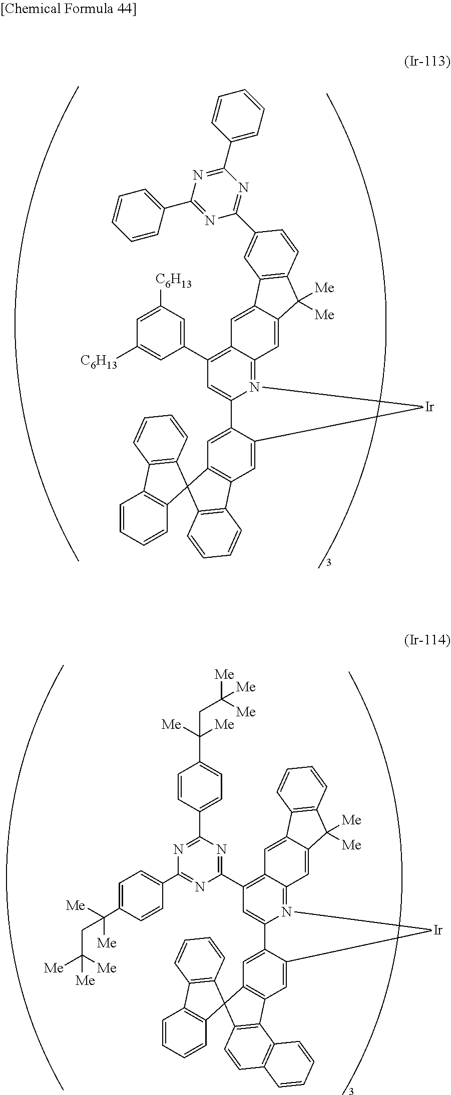

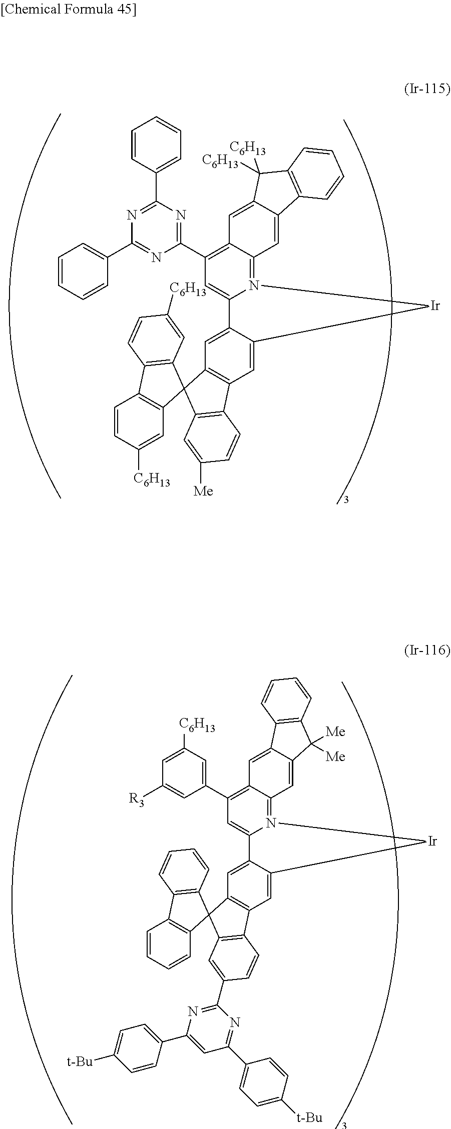

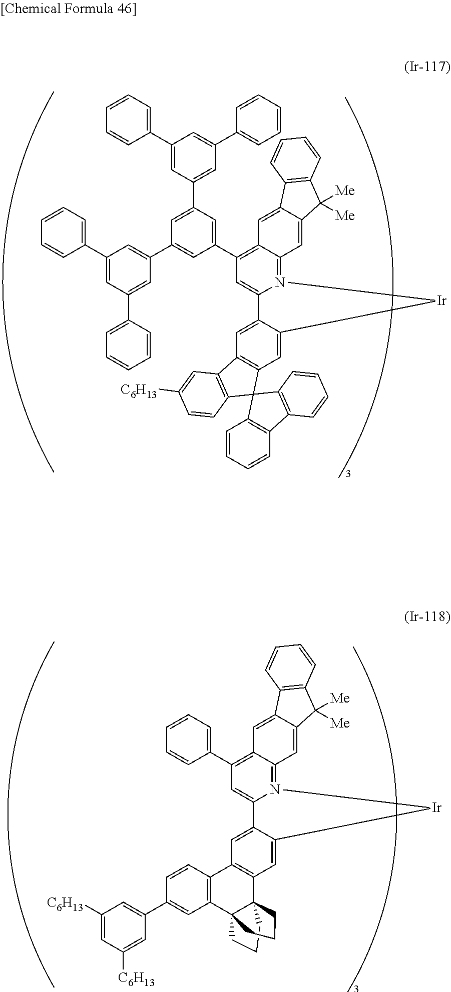

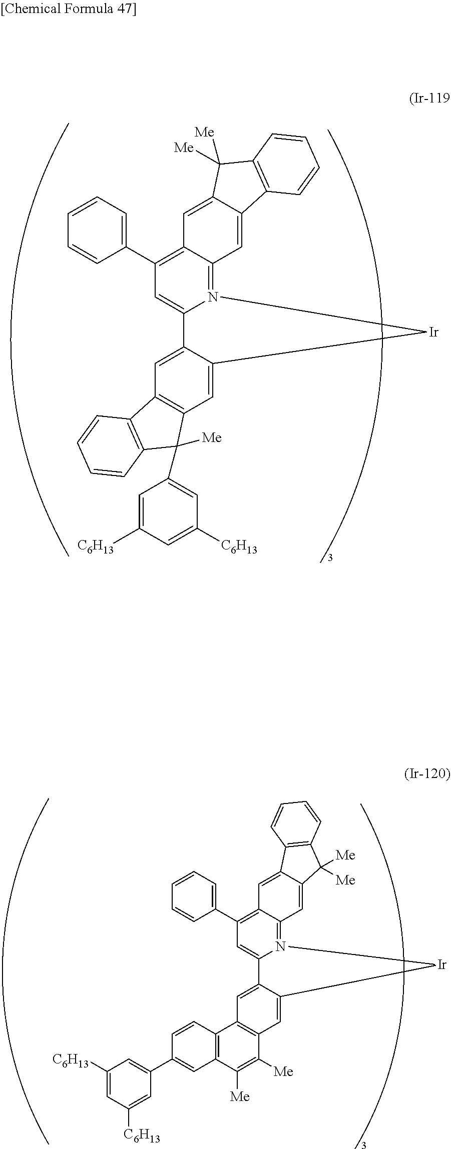

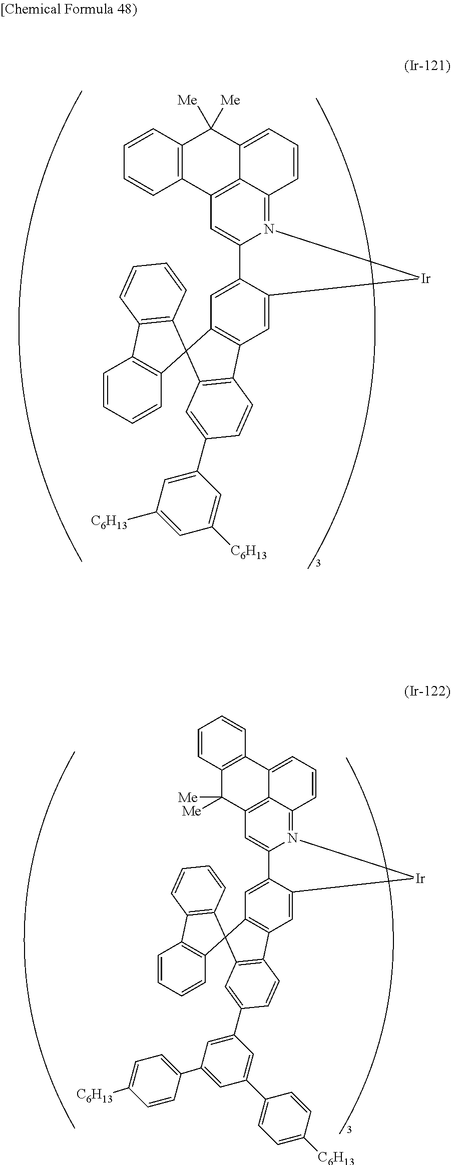

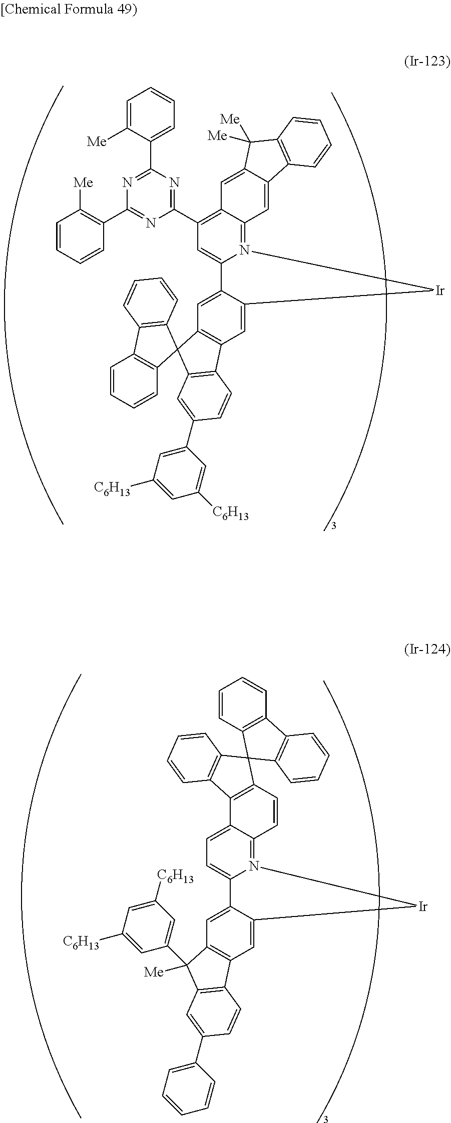

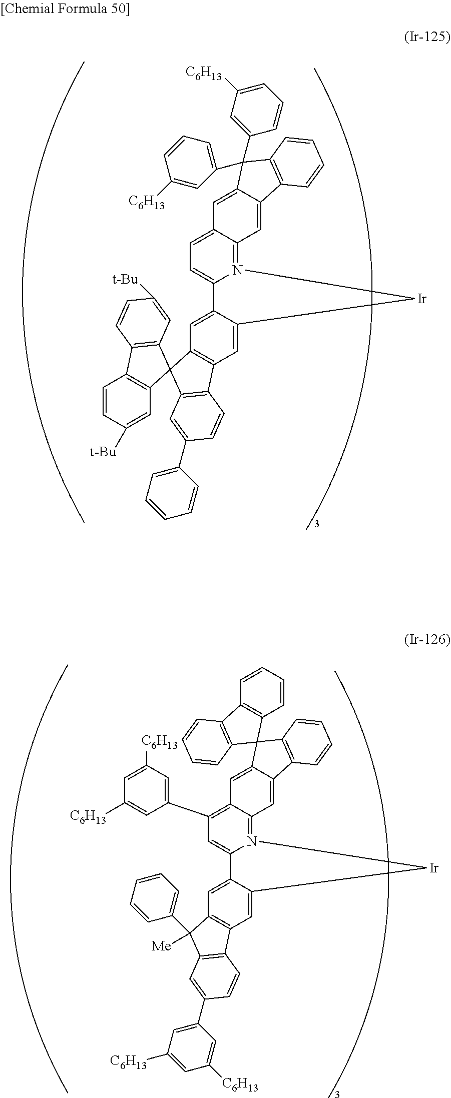

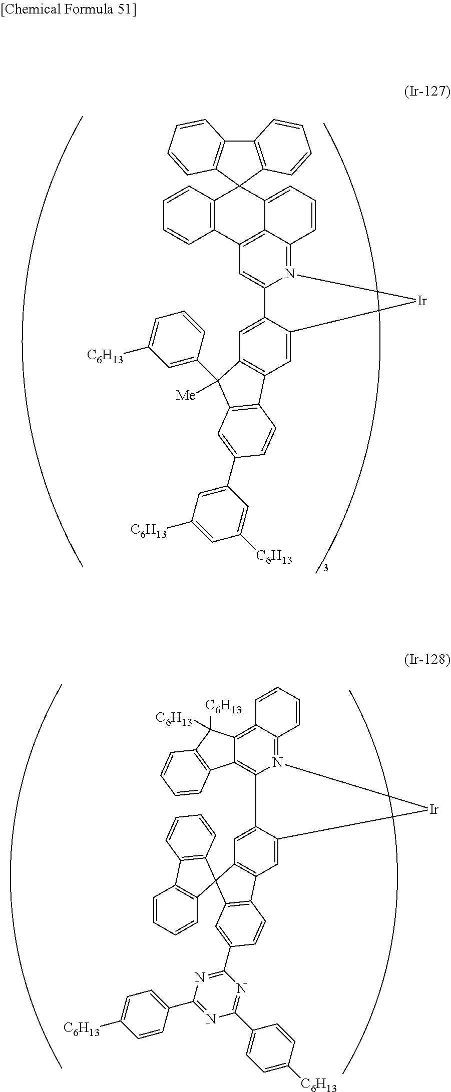

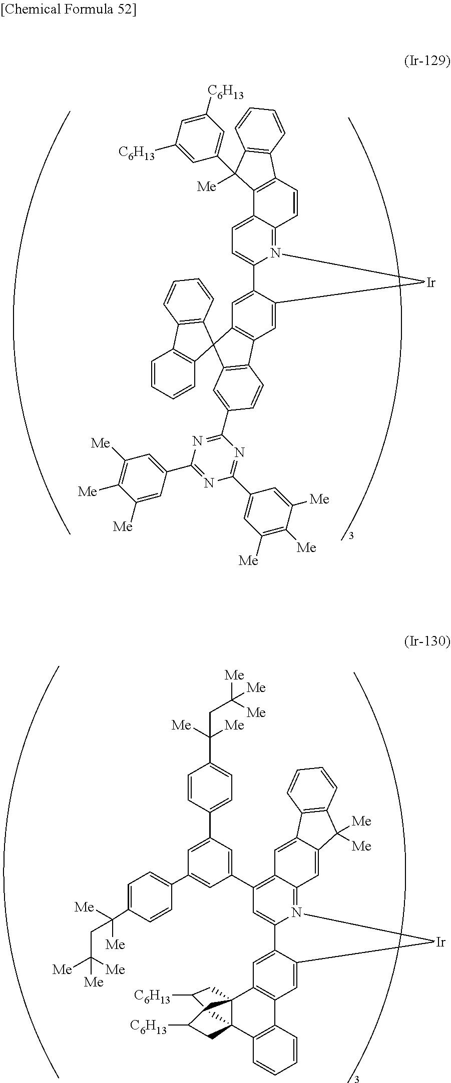

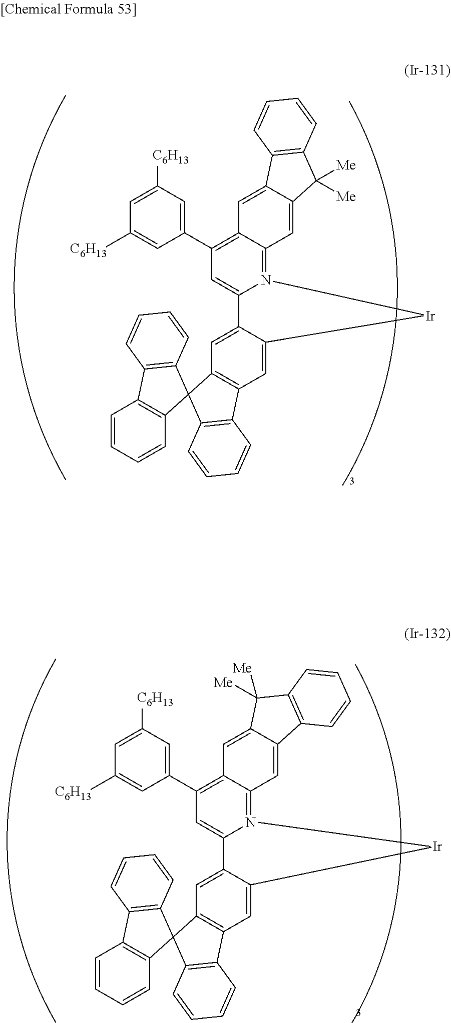

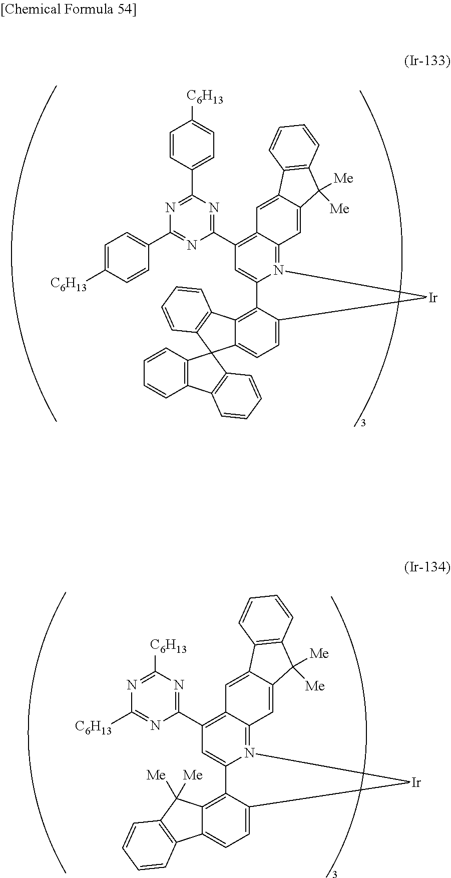

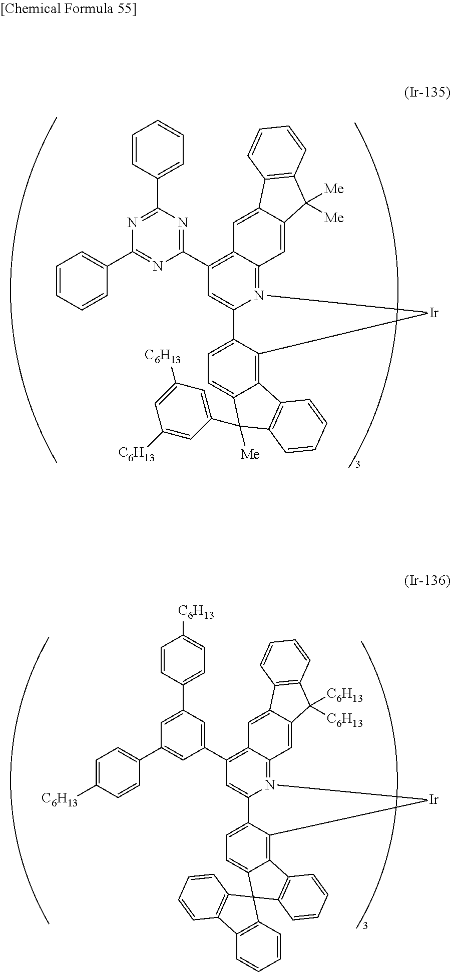

The metal complex of the present embodiment may be used singly or in combination of two or more.

The metal complex of the present embodiment includes a plurality of geometric isomers envisaged, and may be any geometric isomer, and the proportion of a facial geometry is preferably 80% by mol or more, more preferably 90% by mol or more, further more preferably 99% by mol or more, and particularly preferably 100% by mol (namely, not containing other geometric isomers), with respect to the whole metal complex, since the full width at half maximum in the emission spectrum of the metal complex of the present embodiment is more excellent.

### <Production Method of Metal Complex Represented by the Formula (0)>

Methods for producing the above-described metal complex X as a typical metal complex among metal complexes of the present embodiment will be described.

### [Production Method 1]

The metal complex X can be produced, for example, by a method of reacting a compound as a ligand and a metal compound. If necessary, the functional group converting reaction of the ligand of the metal complex may be conducted. Of metal complexes represented by the above-described formula (0), metal complexes other than the metal complex X can also be produced in a like manner.

Of the metal complexes X, those in which M is an iridium atom and n₁ is 2 or 3 can be produced by methods containing, for example,

(i) a step A1 of reacting a compound represented by the following formula (M2-1) and an iridium compound or its hydrate, to synthesize a metal complex represented by the following formula (M2-2), and

(ii) a step B1 of reacting the metal complex represented by the following formula (M2-2), and a compound represented by the following formula (M2-1) or a precursor of a ligand represented by A¹-G¹-A².

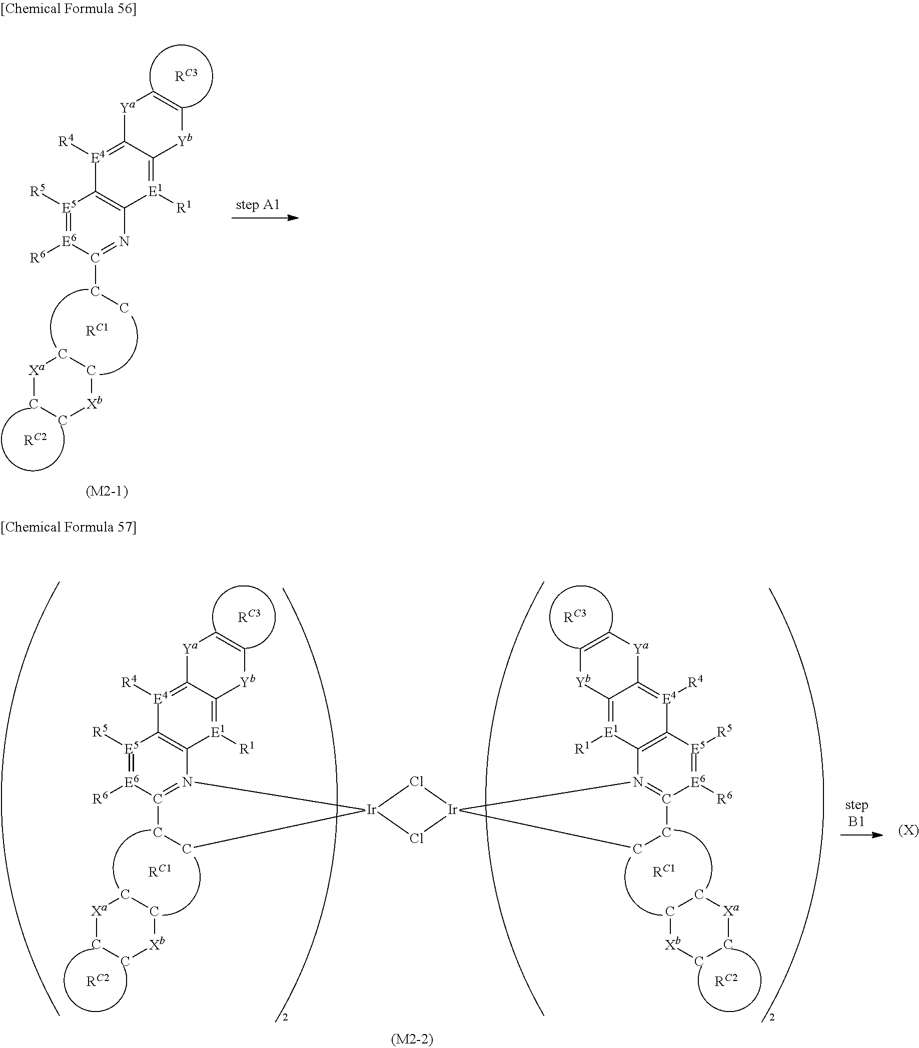

[in the above-described formula (M2-1) and formula (M2-2), R^{C1}, Ring R^{C2}, Ring R^{C3}, X^{a}, X^{b}, Y^{a}, Y^{b}, R¹, R⁴, R⁵, R⁶, E¹, E⁴, E⁵ and E⁶ represent the same meaning as for Ring R^{C1}, Ring R^{C2}, Ring R^{C3}, X^{a}, X^{b}, Y^{a}, Y^{b}, R¹, R⁴, R⁵, R⁶, E¹, E⁴, E⁵ and E⁶ in the above-described formula (0), respectively; provided that, in respective ligands constituting the formula (M2-1) and the formula (M2-2), at least one of Ring R^{C1}, Ring R^{C2} and Ring R^{C3} has a group represented by the above-described formula (D-A), formula (D-B) or formula (D-C)].

In the step A1, the iridium compound includes, for example, iridium chloride, tris(acetylacetonato)iridium (III), chloro (cyclooctadiene)iridium (I) dimer, and iridium(III) acetate, and the iridium compound hydrate includes, for example, iridium chloride.trihydrate.

The step A1 and the step B1 are conducted usually in a solvent. The solvent includes, for example, alcohol solvents such as methanol, ethanol, propanol, ethylene glycol, glycerin, 2-methoxyethanol, 2-ethoxyethanol, and the like; ether solvents such as diethyl ether, tetrahydrofuran, dioxane, cyclopentyl methyl ether, diglyme, and the like; halogen solvents such as methylene chloride, chloroform, and the like; nitrile solvents such as acetonitrile, benzonitrile, and the like; hydrocarbon solvents such as hexane, decalin, toluene, xylene, mesitylene, and the like; amide solvents such as N,N-dimethylformamide, N,N-dimethylacetamide, and the like; and, acetone, dimethyl sulfoxide, and water.

In the step A1 and the step B1, the reaction time is usually 30 minutes to 150 hours, and the reaction temperature is usually between the melting point and the boiling point of a solvent present in the reaction system.

In the step A1, the amount of a compound represented by the formula (M2-1) is usually 2 to 20 mol with respect to 1 mol of an iridium compound or its hydrate.

In the step B1, the amount of a compound represented by the formula (M2-1) or a precursor of a ligand represented by A¹-G¹-A² is usually 1 to 100 mol with respect to 1 mol of a metal complex represented by the formula (M2-2).

In the step B1, the reaction is conducted preferably in the presence of a silver compound such as silver trifluoromethanesulfonate, and the like. When a silver compound is used, its amount is usually 2 to 20 mol with respect to 1 mol of a metal complex represented by the formula (M2-2).

The compound represented by the above-described formula (M2-1) can be synthesized, for example, by a step of coupling-reacting a compound represented by the following formula (M1-3) and a compound represented by the following formula (M1-4), such as in the Suzuki reaction, the Kumada reaction, the Stille reaction, and the like.

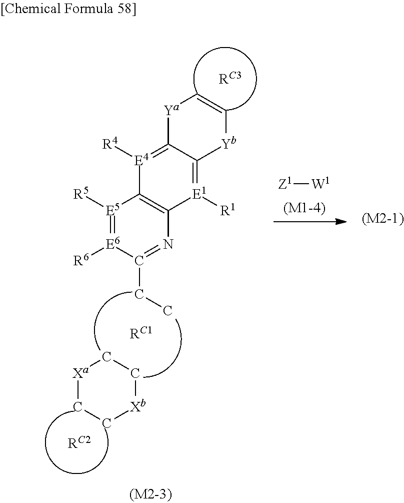

[in the formula (M1-3), Ring R^{C1}, Ring R^{C2}, Ring R^{C3}, X^{a}, X^{b}, Y^{a}, Y^{b}, R¹, R⁴, R⁵, R⁶, E¹, E⁴, E⁵ and E⁶ represent the same meaning as for Ring R^{C1}, Ring R^{C2}, Ring R^{C3}, X^{a}, X^{b}, Y^{a}, Y^{b}, R¹, R⁴, R⁵, R⁶, E¹, E⁴, E⁵ and E⁶ in the above-described formula (0), respectively; provided that, at least one of Ring R^{C1}, Ring R^{C2} and Ring R^{C3} has a group represented by —B(OR^{W1})₂, an alkylsulfonyloxy group, a cycloalkylsulfonyloxy group, an arylsulfonyloxy group, a chlorine atom, a bromine atom or an iodine atom, as a substituent, instead of the group represented by the formula (D-A), the formula (D-B) or the formula (D-C) in the above-described formula (M2-1);

R^{W1} represents a hydrogen atom, an alkyl group, a cycloalkyl group, an aryl group or an amino group, and these groups optionally have a substituent. The plurality of R^{W1} may be the same or different and may be combined together to form a ring structure together with an oxygen atom to which they are attached].

[in the formula (M1-4), Z¹ represents a group represented by the above-described formula (D-A), formula (D-B) or formula (D-C). W¹ represents a group represented by —B(OR^{W1})₂, an alkylsulfonyloxy group, a cycloalkylsulfonyloxy group, an arylsulfonyloxy group, a chlorine atom, a bromine atom or an iodine atom, and these groups optionally have a substituent; R^{W1} represents the same meaning as for R^{W1} in the formula (M1-3)].

The group represented by —B(OR^{W1})₂ includes, for example, groups represented by the following formula (W-1) to formula (W-10).

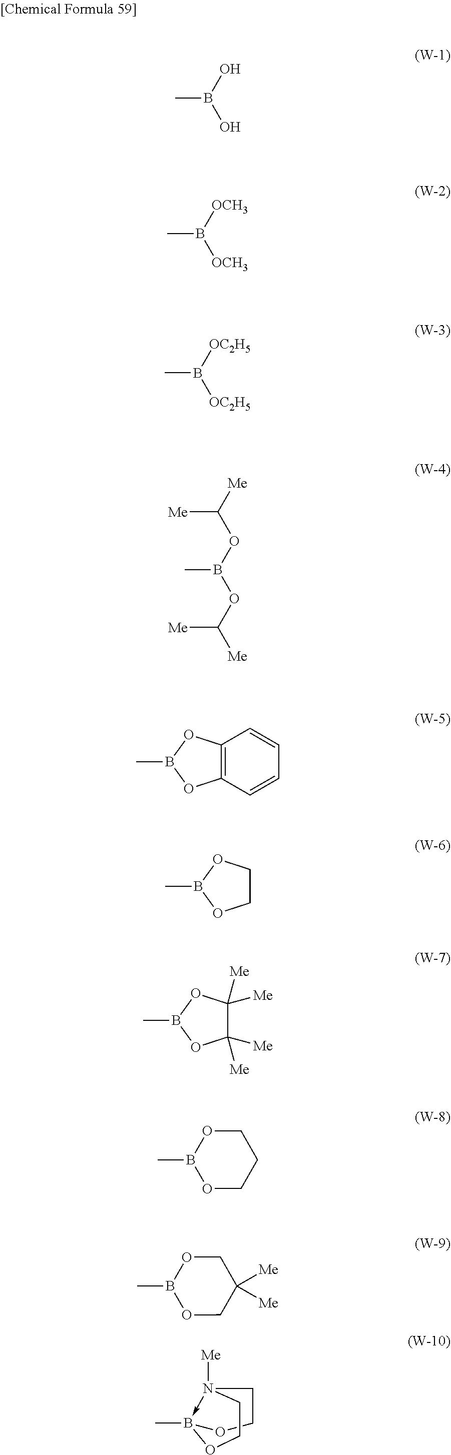

The alkylsulfonyloxy group represented by W¹ includes, for example, a methanesulfonyloxy group, an ethanesulfonyloxy group, and a trifluoromethanesulfonyloxy group.

The arylsulfonyloxy group represented by W¹ includes, for example, a p-toluenesulfonyloxy group.

W¹ is more preferably a chlorine atom, a bromine atom or a group represented by the above-described formula (W-7), since a coupling reaction of a compound represented by the above-described formula (M1-4) and a metal complex represented by the above-described formula (M1-3) proceeds easily.

The alkylsulfonyloxy group, the cycloalkylsulfonyloxy group and the arylsulfonyloxy group as the substituent which at least one of Ring R^{C1}, Ring R^{C2} and Ring R^{C3} has instead of the group represented by the above-described formula (D-A), formula (D-B) or formula (D-C) represent the same meaning as for the alkylsulfonyloxy group, the cycloalkylsulfonyloxy group and the arylsulfonyloxy group represented by W¹, respectively.

Z¹ is preferably a group represented by the above-described formula (D-A), and more preferably a group represented by the above-described formula (D-A1) to formula (D-A3).

The coupling reaction of a compound represented by the above-described formula (M1-3) and a compound represented by the above-described formula (M1-4) is usually conducted in a solvent. The solvent, the reaction time and the reaction temperature to be used may be the same as those explained for the step A1 and the step B1.

In the coupling reaction of a compound represented by the above-described formula (M1-3) and a compound represented by the above-described formula (M1-4), the amount of a compound represented by the formula (M1-4) is usually 0.05 to 20 mol with respect to 1 mol of a compound represented by the formula (M1-3).

The compound represented by the above-described formula (M1-4) includes, for example, compounds in which Z¹ is a group represented by the above-described formula (D-A1) to formula (D-A3) and W¹ is a group represented by —B(OR^{W1})₂, a trifluoromethanesulfonyloxy group, a bromine atom or an iodine atom.

A compound represented by the following formula (M1-4-1) which is one embodiment of the compound represented by the above-described formula (M1-4) can be synthesized, for example, by the following method.

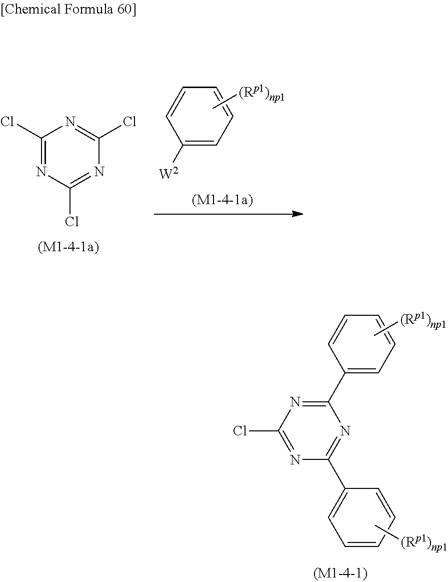

[wherein,

R^{p1} represents an alkyl group, a cycloalkyl group, an alkoxy group, or a cycloalkoxy group; when a plurality of R^{p1} are present, they may be the same or different; np1 represents an integer of 0 to 5; a plurality of np1 may be the same or different;

W² represents a group represented by —B(OR^{W1})₂, an alkylsulfonyloxy group, a cycloalkylsulfonyloxy group, an arylsulfonyloxy group, a chlorine atom, a bromine atom or an iodine atom, and these groups optionally have a substituent].

The compound represented by the above-described formula (M1-4-1) can be produced, for example, by coupling-reacting a compound represented by the above-described formula (M1-4-1a) and a compound represented by the above-described formula (M1-4-1b). This coupling reaction may be the same reaction as explained for the compound represented by the above-described formula (M2-1).

A compound represented by the following formula (M1-4-2) which is one embodiment of the compound represented by the above-described formula (M1-4) can be synthesized, for example, by the following method.

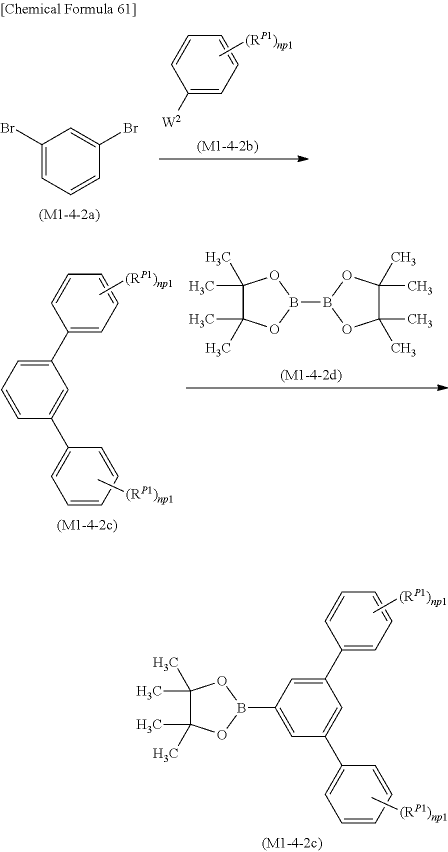

[wherein, R^{p1}, n^{p1} and W² represent the same meaning as for R^{p1}, n^{p1} and W² in the above-described formula M (1-4-1) and formula M (1-4-1b)].

The compound represented by the above-described formula (M1-4-2c) can be produced, for example, by coupling-reacting a compound represented by the above-described formula (M1-4-2a) and a compound represented by the above-described formula (M1-4-2b). This coupling reaction may be the same reaction as explained for the compound represented by the above-described formula (M2-1).

The compound represented by the above-described formula (M1-4-2) can be synthesized, for example, by Ishiyama-Miyaura-Hartwig-reacting a compound represented by the above-described formula (M1-4-2c) and a compound represented by the above-described formula (M1-4-2d).

The compound represented by the above-described formula (M1-3) can be produced, for example, by coupling-reacting a compound represented by the following formula (M1-5) and a compound represented by the following formula (M1-6). This coupling reaction may be the same reaction as explained for the compound represented by the above-described formula (M2-1).

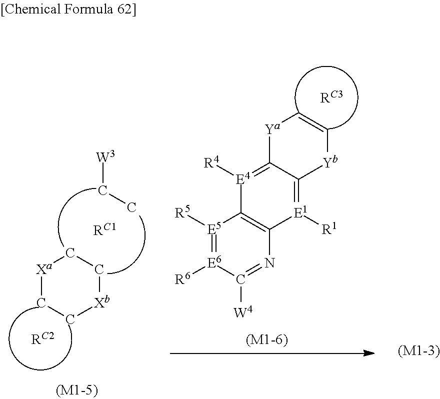

[in the formula (M1-5), Ring R^{C1}, Ring R^{C2}, X^{a} and X^{b} represent the same meaning as for Ring R^{C1}, Ring R^{C2}, X^{a} and X^{b} in the above-described formula (0), respectively;

in the formula (M1-6), Ring R^{C3}, Y^{a}, Y^{b}, R¹, R⁴, R⁵, R⁶, E¹, E⁴, E⁵ and E⁶ represent the same meaning as for Ring R^{C3}, Y^{a}, Y^{b}, R¹, R⁴, R⁵, R⁶, E¹, E⁴, E⁵ and E⁶ in the above-described formula (0), respectively; provided that, at least one of Ring R^{C1}, Ring R^{C2} and Ring R^{C3} has a group represented by —B(OR^{W1})₂, an alkylsulfonyloxy group, a cycloalkylsulfonyloxy group, an arylsulfonyloxy group, chlorine atom, a bromine atom or an iodine atom, as a substituent, instead of the group represented by the above-described formula (D-A), formula (D-B) or formula (D-C);

W³ and W⁴ each independently represent a group represented by —B(OR^{W1})₂, an alkylsulfonyloxy group, a cycloalkylsulfonyloxy group, an arylsulfonyloxy group, a chlorine atom, a bromine atom or an iodine atom, and these groups optionally have a substituent].

Of the metal complexes X, those in which M is an iridium atom and n₁ is 1 can be produced by methods containing, for example,

(i) a step A1′ of reacting a precursor of a ligand represented by A¹-G¹-A², and an iridium compound or its hydrate, and

(ii) a step B1′ of reacting the metal complex obtained in the step A1′, and a compound represented by the above-described formula (M2-1).

The step A1′ and the step B1′ can be carried out with reference to the step A1 and the step B1, respectively.

### [Production Method 2]

The above-described metal complex X can also be produced, for example, by a method of reacting a precursor of a metal complex and a precursor of a ligand of a metal complex.

The metal complex X can be produced, for example, by coupling-reacting a compound represented by the above-described formula (M1-4), and a metal complex represented by the following formula (M1-7) This coupling reaction may be the same reaction as explained for the compound represented by the above-described formula (M2-1).

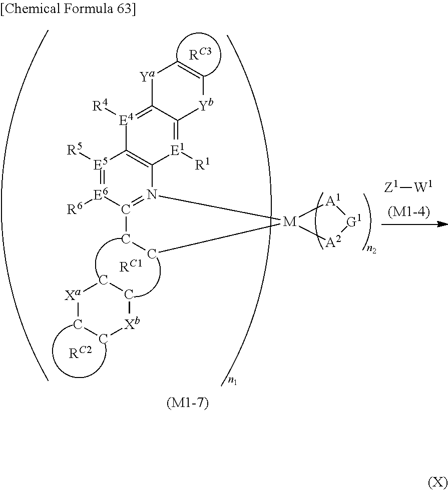

[in the formula (M1-7), n₁, n₂, Ring R^{C1}, Ring R^{C2}, Ring R^{C3}, X^{a}, X^{b}, Y^{a}, Y^{b}, R¹, R⁴, R⁵, R⁶, E¹, E⁴, E⁵, E⁶ and A¹-G¹-A² represent the same meaning as for n₁, n₂, Ring R^{C1}, Ring R^{C2}, Ring R^{C3}, X^{a}, X^{b}, Y^{a}, Y^{b}, R¹, R⁴, R⁵, R⁶, E¹, E⁴, E⁵, E⁶ and A¹-G¹-A² in the above-described formula (0), respectively; provided that, in respective ligands or one ligand of which number is defined by index n¹, at least one of Ring R^{C1}, Ring R^{C2} and Ring R^{C3} has a group represented by —B(OR^{W1})₂, an alkylsulfonyloxy group, a cycloalkylsulfonyloxy group, an arylsulfonyloxy group, a chlorine atom, a bromine atom or an iodine atom, as a substituent, instead of the group represented by the above-described formula (D-A), formula (D-B) or formula (D-C);

R^{W1} represents a hydrogen atom, an alkyl group, a cycloalkyl group, an aryl group or an amino group, and these groups optionally have a substituent. The plurality of R^{W1} may be the same or different and may be combined together to form a ring structure together with an oxygen atom to which they are attached].

The metal complex represented by the above-described formula (M1-7) can be synthesized, for example, by using a compound represented by the above-described formula (M1-3) instead of the compound represented by the above-described formula (M2-1) in the step A1 and the step B1 in “Production method 1” described above.

The compound, the catalyst and the solvent used in each reaction explained in “<Production method of metal complex represented by the formula (0)>” describe above may be used each singly or in combination of two or more.

### <Composition>

The composition of the present embodiment contains at least one material selected from the group consisting of a host material, a light emitting material different from the metal complex of the present embodiment, an antioxidant and a solvent, and the metal complex of the present embodiment.

In the composition of the present embodiment, the metal complex of the present embodiment may be contained singly or in combination of two or more.

### [Host Material]

“The host material” is a material having at least one function selected from hole injectability, hole transportability, electron injectability and electron transportability. The host material can constitute a composition together with a guest material. The guest material includes, for example, the metal complex of the present embodiment. By converting the metal complex of the present embodiment into a composition with the host material, the luminescence lifetime of a light emitting device obtained using the metal complex of the present embodiment is more excellent. In the composition of the present embodiment, the host material may be contained singly or in combination of two or more.

In a composition containing the metal complex of the present embodiment and the host material, the content of the metal complex of the present embodiment is usually 0.01 parts by mass to 80 parts by mass, preferably 0.05 parts by mass to 40 parts by mass, more preferably 0.1 parts by mass to 20 parts by mass, and further preferably 1 part by mass to 20 parts by mass, when the sum the metal complex of the present embodiment and the host material is taken as 100 parts by mass.

It is preferable that the lowest excited triplet state (T₁) of the host material has energy level equivalent to or higher than the energy level of the lowest excited triplet state (T₁) of the metal complex of the present embodiment, since the luminescence lifetime of a light emitting device obtained using the composition of the present embodiment is more excellent.

The host material is preferably one having solubility in a solvent capable of dissolving the metal complex of the present embodiment, since a light emitting device obtained using the composition of the present embodiment can be fabricated by a solution applying process.

The host material is classified into a low molecular compound used for the host material (hereinafter, referred to also as “low molecular host”), and a polymer compound used for the host material (hereinafter, referred to also as “polymer host”). Examples of the host material include hole injection materials, hole transporting materials, electron injection materials, and electron transporting materials described later, in addition to the following preferred low molecular hosts and polymer hosts.

### <Low Molecular Host>

The low molecular host is preferably a compound represented by the following formula (H-1).

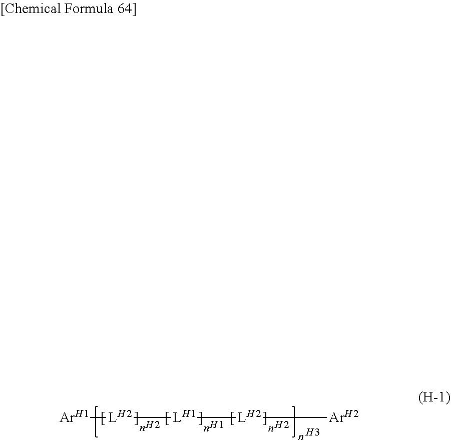

[wherein,

Ar^{H1} and Ar^{H2} each independently represent an aryl group or a monovalent heterocyclic ring group, and these groups optionally have a substituent;

n^{H1} and n^{H2} each independently represent 0 or 1. When a plurality of n^{H1} are present, they may be the same or different; when a plurality of n^{H2} are present, the plurality of n^{H2} may be the same or different;

n^{H3} represents an integer of 0 or more;

L^{H1} represents an arylene group, a divalent heterocyclic ring group, or a group represented by —[C(R^{H11})₂]n^{H11}-, and these groups optionally have a substituent; when a plurality of L^{H1} are present, they may be the same or different; n^{H11} represents an integer of 1 or more and 10 or less; R^{H11} represents a hydrogen atom, an alkyl group, a cycloalkyl group, an alkoxy group, a cycloalkoxy group, an aryl group or a monovalent heterocyclic ring group, and these groups optionally have a substituent; a plurality of R^{H11} may be the same or different and may be combined together to form a ring together with carbon atoms to which they are attached;

L^{H2} represents a group represented by —N(-L^{H21}-R^{H21})—; when a plurality of L^{H2} are present, the plurality of L^{H2} may be the same or different; L^{H21} is a single bond, an arylene group or a divalent heterocyclic ring group, and these groups optionally have a substituent; R^{H21} represents a hydrogen atom, an alkyl group, a cycloalkyl group, an aryl group or a monovalent heterocyclic ring group, and these groups optionally have a substituent].

Ar^{H1} and Ar^{H2} represent preferably a phenyl group, a fluorenyl group, a spirobifluorenyl group, a pyridyl group, a pyrimidinyl group, a triazinyl group, a quinolinyl group, an isoquinolinyl group, a thienyl group, a benzothienyl group, a dibenzothienyl group, a furyl group, a benzofuryl group, a dibenzofuryl group, a pyrrolyl group, an indolyl group, an azaindolyl group, a carbazolyl group, an azacarbazolyl group, a diazacarbazolyl group, a phenoxazinyl group or a phenothiazinyl group, more preferably a phenyl group, a spirobifluorenyl group, a pyridyl group, a pyrimidinyl group, a triazinyl group, a dibenzothienyl group, a dibenzofuryl group, a carbazolyl group or an azacarbazolyl group, further preferably a phenyl group, a pyridyl group, a carbazolyl group or an azacarbazolyl group, particularly preferably a group represented by the above-described formula (TDA-1) or formula (TDA-3), and especially preferably a group represented by the above-described formula (TDA-3), and these groups optionally have a substituent.

The substituent which Ar^{H1} and Ar^{H2} optionally have is preferably a halogen atom, an alkyl group, a cycloalkyl group, an alkoxy group, a cycloalkoxy group, an aryl group or a monovalent heterocyclic ring group, more preferably an alkyl group, an alkoxy group or a cycloalkoxy group, and further preferably an alkyl group or a cycloalkoxy group, and these groups optionally further have a substituent.

n^{H1} is preferably 1. n^{H2} is preferably 0.

n^{H3} is usually an integer of 0 or more and 10 or less, preferably an integer of 0 or more and 5 or less, further preferably an integer of 1 or more and 3 or less, and particularly preferably 1.

n^{H11} is preferably an integer of 1 or more and 5 or less, more preferably an integer of 1 or more and 3 or less, and further preferably 1.

R^{H11} is preferably a hydrogen atom, an alkyl group, a cycloalkyl group, an aryl group or a monovalent heterocyclic ring group, more preferably a hydrogen atom, an alkyl group or a cycloalkyl group, and further preferably a hydrogen atom or an alkyl group, and these groups optionally have a substituent.

L^{H1} is preferably an arylene group or a divalent heterocyclic ring group.

L^{H1} is preferably a group represented by the above-described formula (A-1) to formula (A-3), formula (A-8) to formula (A-10), formula (AA-1) to formula (AA-6), formula (AA-10) to formula (AA-21) or formula (AA-24) to formula (AA-34), more preferably a group represented by the above-described formula (A-1), formula (A-2), formula (A-8), formula (A-9), formula (AA-1) to formula (AA-4), formula (AA-10) to formula (AA-15) or formula (AA-29) to formula (AA-34), further preferably a group represented by the above-described formula (A-1), formula (A-2), formula (A-8), formula (A-9), formula (AA-2), formula (AA-4), formula (AA-10) to formula (AA-15), particularly preferably a group represented by the above-described formula (A-1), formula (A-2), formula (A-8), formula (AA-2), formula (AA-4), formula (AA-10), formula (AA-12) or formula (AA-14), and especially preferably a group represented by the above-described formula (A-1), formula (A-2), formula (AA-2), formula (AA-4) or formula (AA-14).

The substituent which L^{H1} optionally has is preferably a halogen atom, an alkyl group, a cycloalkyl group, an alkoxy group, a cycloalkoxy group, an aryl group or monovalent heterocyclic ring group, more preferably an alkyl group, an alkoxy group, an aryl group or a monovalent heterocyclic ring group, and further preferably an alkyl group, an aryl group or a monovalent heterocyclic ring group, and these groups optionally further have a substituent.

L^{H21} is preferably a single bond or an arylene group, and more preferably a single bond, and this arylene group optionally has a substituent.

The examples and the preferable range of the arylene group or the divalent heterocyclic ring group represented by L^{H21} are the same as the examples and the preferable range of, the arylene group or the divalent heterocyclic ring group represented by L^{H1}.

R^{H21} is preferably an aryl group or a monovalent heterocyclic ring group, and these groups optionally have a substituent.

The examples and the preferable ranges of the aryl group and the monovalent heterocyclic ring group represented by R^{H21} are the same as the examples and the preferable ranges of the aryl group and the monovalent heterocyclic ring group represented by Ar^{H1} and Ar^{H2}.

The examples and the preferable range of the substituent which R^{H21} optionally has are the same as the examples and the preferable range of the substituent which Ar^{H1} and Ar^{H2} optionally have.

The compound represented by the above-described formula (H-1) is preferably a compound represented by the following formula (H-2).

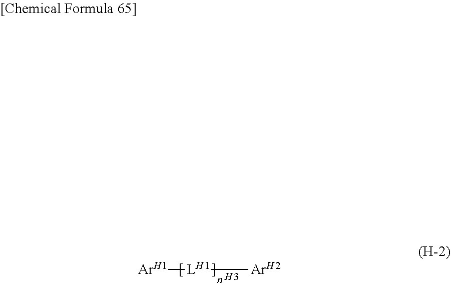

[wherein, Ar^{H1}, Ar^{H2}, n^{H3} and L^{H1} represent the same meaning as for Ar^{H1}, Ar^{H2}, n^{H3} and L^{H1} in the above-described formula (H-1), respectively].

Specific examples of the low molecular host include compounds represented by the following formulae.

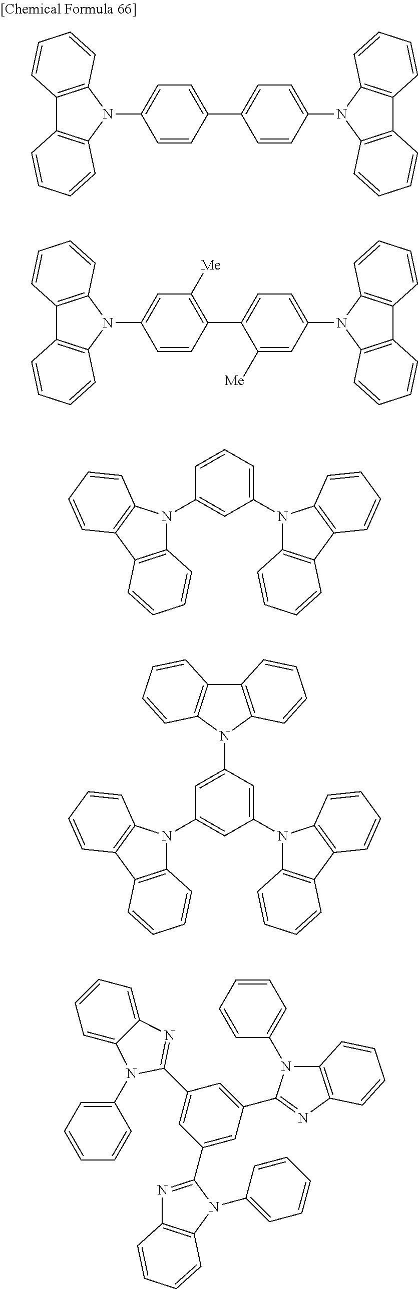

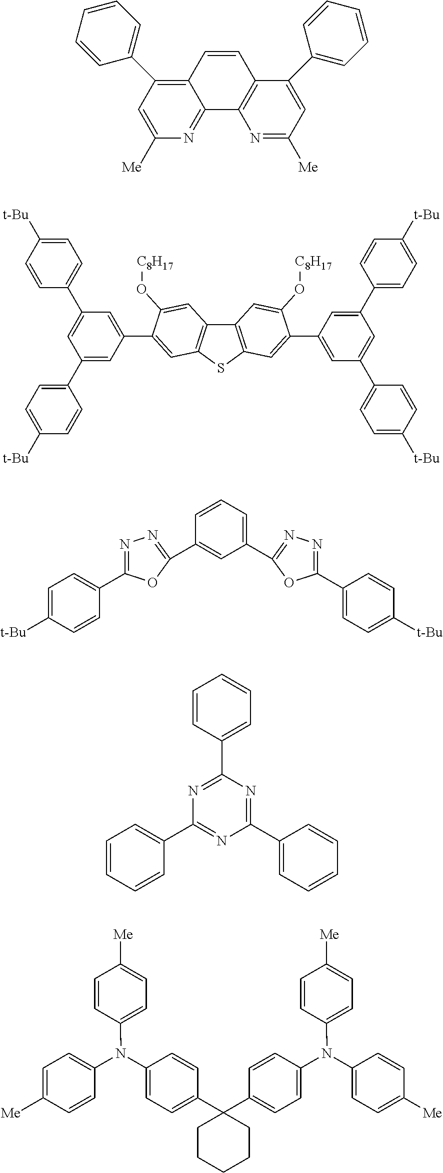

### <Polymer Host>

The polymer host includes, for example, polymer compounds as the hole transporting material described later, and polymer compounds as the electron transporting material described later.

The polymer compound which is preferred as the host material will be illustrated.

The polymer host is preferably a polymer compound containing a constitutional unit represented by the following formula (Y).

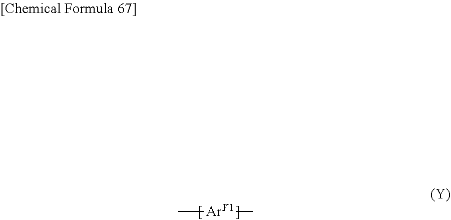

[wherein, Ar^{Y1} represents an arylene group, a divalent heterocyclic ring group, or a divalent group in which an arylene group and a divalent heterocyclic ring group are bonded directly, and these groups optionally have a substituent].

The arylene group represented by Ar^{Y1} is more preferably a group represented by the above-described formula (A-1), formula (A-2), formula (A-6) to formula (A-10), formula (A-19) or formula (A-20), and further preferably a group represented by the above-described formula (A-1), formula (A-2), formula (A-7), formula (A-9) or formula (A-19), and these groups optionally have a substituent.

The divalent heterocyclic ring group represented by Ar^{Y1} is more preferably a group represented by the above-described formula (AA-1) to formula (AA-4), formula (AA-10) to formula (AA-15), formula (AA-18) to formula (AA-21), formula (A-33) or formula (A-34), and further preferably a group represented by the above-described formula (AA-4), formula (AA-10), formula (AA-12), formula (AA-14) or formula (AA-33), and these groups optionally have a substituent.

The more preferable ranges and the further preferable ranges of an arylene group and a divalent heterocyclic ring group in the divalent group in which an arylene group and a divalent heterocyclic ring group are bonded directly represented by Ar^{Y1} are the same as the more preferable ranges and the further preferable ranges of the arylene group and the divalent heterocyclic ring group represented by Ar^{Y1} described above, respectively.

“The divalent group in which an arylene group and a divalent heterocyclic ring group are bonded directly” includes, for example, groups represented by the following formulae, and these optionally have a substituent.

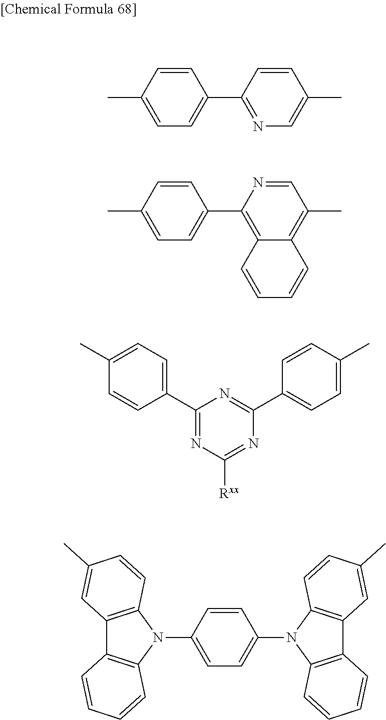

[wherein, R^{XX} represents a hydrogen atom, an alkyl group, a cycloalkyl group, an aryl group or a monovalent heterocyclic ring group, and these groups optionally have a substituent].

R^{XX} is preferably an alkyl group, a cycloalkyl group or an aryl group, and these groups optionally have a substituent.

The substituent which a group represented by Ar^{Y1} optionally has is preferably an alkyl group, a cycloalkyl group or an aryl group, and these groups optionally further have a substituent.

The constitutional unit represented by the above-described formula (Y) includes, for example, constitutional units represented by the following formula (Y-1), formula (Y-2), and formula (Y-4) to formula (Y-10).

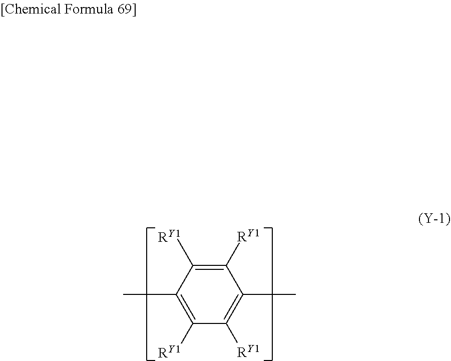

[wherein, R^{Y1} represents a hydrogen atom, an alkyl group, a cycloalkyl group, an alkoxy group, a cycloalkoxy group, an aryl group or a monovalent heterocyclic ring group, and these groups optionally have a substituent; a plurality of R^{Y1} may be the same or different, and adjacent groups R^{Y1} may be combined together to form a ring together with carbon atoms to which they are attached].

R^{Y1} is preferably a hydrogen atom, an alkyl group, a cycloalkyl group or an aryl group, and these groups optionally have a substituent.

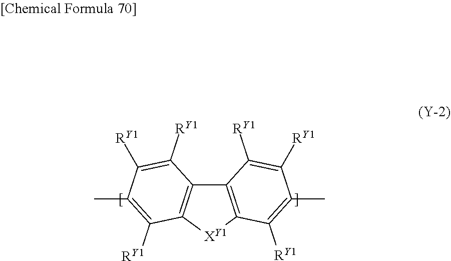

[wherein, R^{Y1} represents the same meaning as for R^{Y1} in the above-described formula (Y-1); X^{Y1} represents a group represented by —C(R^{Y2})₂—, —C(R^{Y2})═C(R^{Y2})—, or C(R^{Y2})₂—C(R^{Y2})₂—; R^{Y2} represents a hydrogen atom, an alkyl group, a cycloalkyl group, an alkoxy group, a cycloalkoxy group, an aryl group or a monovalent heterocyclic ring group, and these groups optionally have a substituent; a plurality of R^{Y2} may be the same or different, and the plurality of R^{Y2} may be combined together to form a ring together with carbon atoms to which they are attached].

R^{Y2} is preferably an alkyl group, a cycloalkyl group, an aryl group or a monovalent heterocyclic ring group, and more preferably an alkyl group, a cycloalkyl group or an aryl group, and these groups optionally have a substituent.

In X^{Y1}, the combination of two groups R^{Y2} in the group represented by —C(R^{Y2})₂— is preferably a combination in which both are alkyl groups or cycloalkyl groups, both are aryl groups, both are monovalent heterocyclic ring groups, or one is an alkyl group or a cycloalkyl group and the other is an aryl group or a monovalent heterocyclic ring group, and more preferably a combination in which one is an alkyl group or a cycloalkyl group and the other is an aryl group, and these groups optionally have a substituent. Two groups R^{Y2} may be combined together to form a ring together with atoms to which they are attached, and when R^{Y2} forms a ring, the group represented by —C(R^{Y2})₂— is preferably a group represented by the following formula (Y-A1) to formula (Y-A5), and more preferably a group represented by the following formula (Y-A4), and these groups optionally have a substituent.

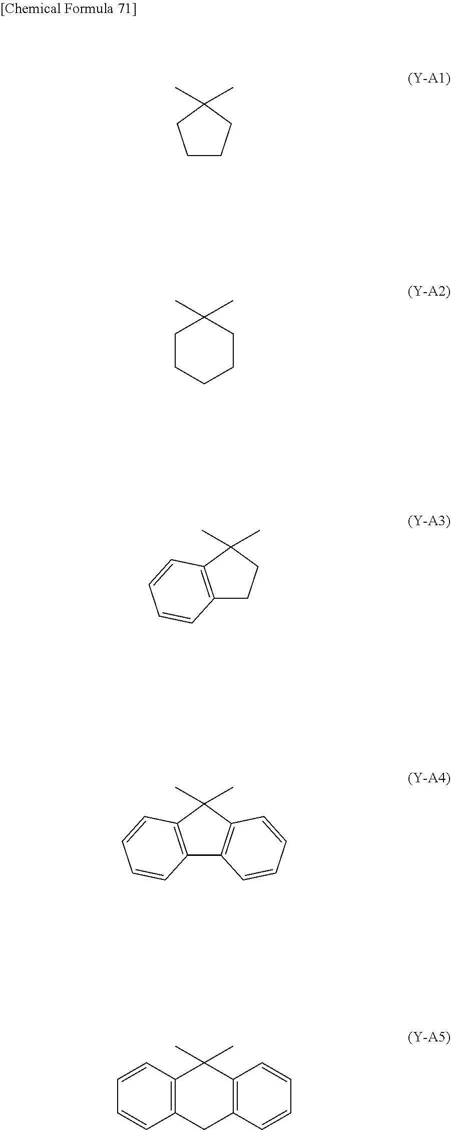

In X^{Y1}, the combination of two groups R^{Y2} in the group represented by —C(R^{Y2})═C(R^{Y2})— is preferably a combination in which both are alkyl groups or cycloalkyl groups, or one is an alkyl group or a cycloalkyl group and the other is an aryl group, and these groups optionally have a substituent.

In X^{Y1}, four groups R^{Y2} in the group represented by —C(R^{Y2})₂—C(R^{Y2})₂— are preferably alkyl groups or cycloalkyl groups optionally having a substituent. A plurality of R^{Y2} may be combined together to form a ring together with atoms to which they are attached, and when R^{Y2} forms a ring, the group represented by —C(R^{Y2})₂—C(R^{Y2})₂— is preferably a group represented by the following formula (Y-B1) to formula (Y-B5), and more preferably a group represented by the following formula (Y-B3), and these groups optionally have a substituent.

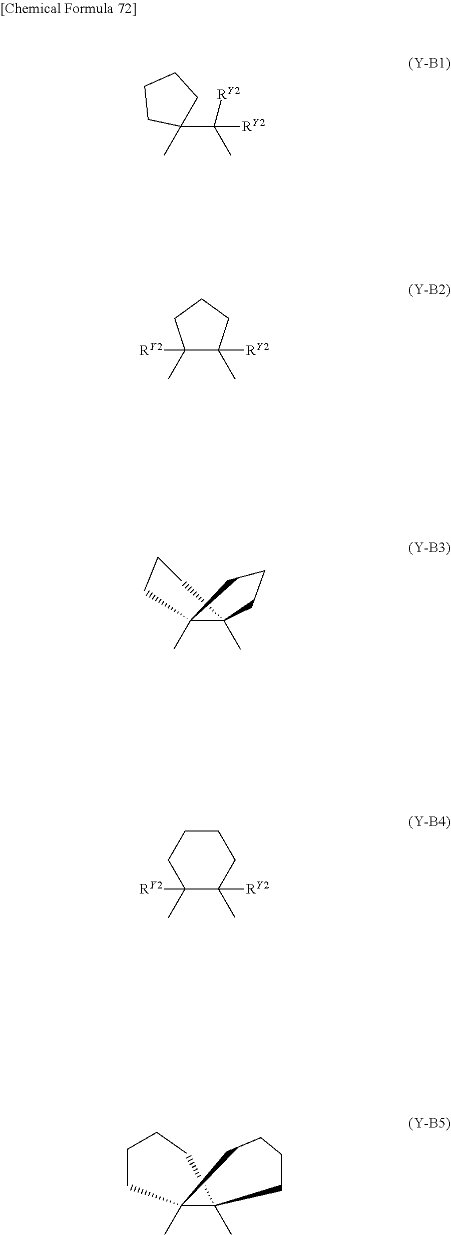

[wherein, R^{Y2} represents the same meaning as for R^{Y2} in the above-described formula (Y-2)].

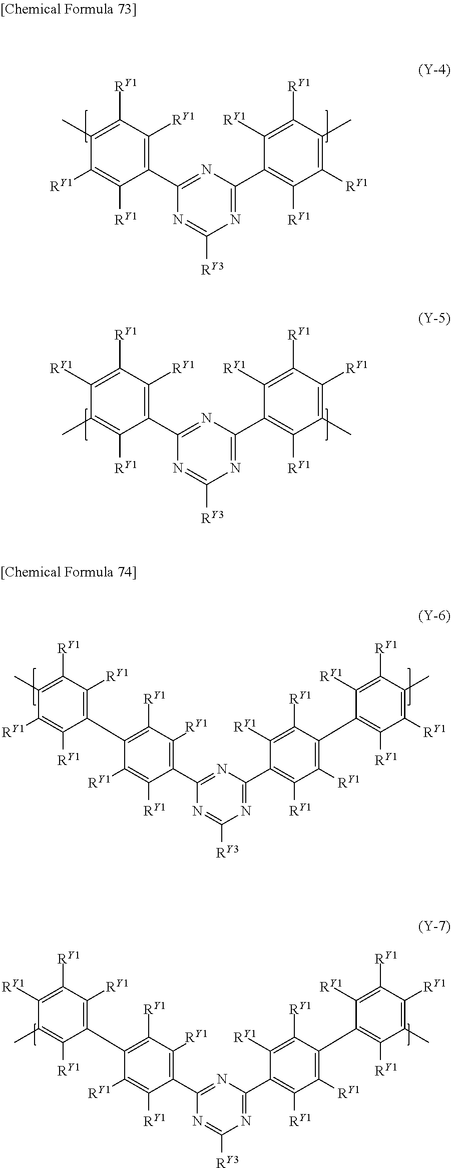

[wherein, R^{Y1} represents the same meaning as for R^{Y1} in the above-described formula (Y-1); R^{Y3} represents a hydrogen atom, an alkyl group, a cycloalkyl group, an alkoxy group, a cycloalkoxy group, an aryl group or a monovalent heterocyclic ring group, and these groups optionally have a substituent].

R^{Y3} is preferably an alkyl group, a cycloalkyl group, an alkoxy group, a cycloalkoxy group, an aryl group or a monovalent heterocyclic ring group, and more preferably an aryl group, and these groups optionally have a substituent.

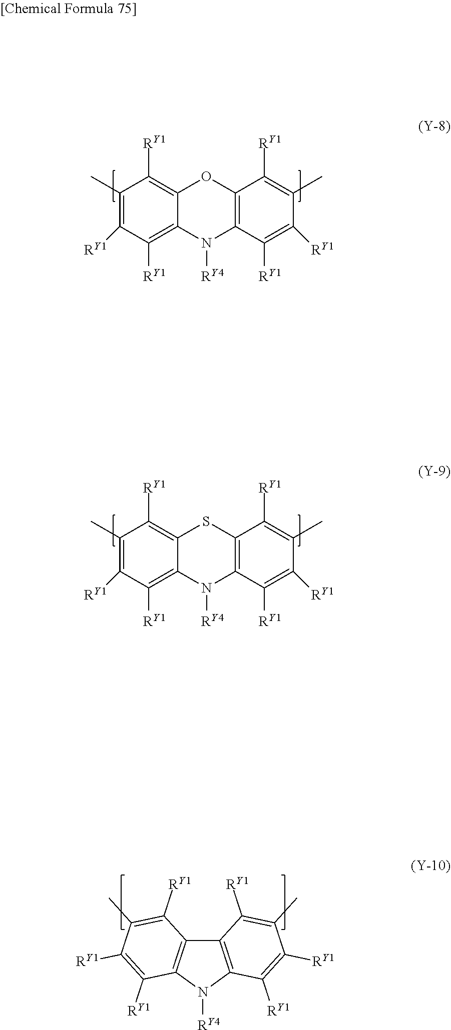

[wherein, R^{Y1} represents the same meaning as for R^{Y1} in the above-described formula (Y-1); R^{Y4} represents a hydrogen atom, an alkyl group, a cycloalkyl group, an alkoxy group, a cycloalkoxy group, an aryl group or a monovalent heterocyclic ring group, and these groups optionally have a substituent].

R^{Y4} is preferably an alkyl group, a cycloalkyl group, an alkoxy group, a cycloalkoxy group, an aryl group or a monovalent heterocyclic ring group, and more preferably an aryl group, and these groups optionally have a substituent.

The constitutional unit represented by the above-described formula (Y) includes, for example, constitutional units composed of an arylene group represented by the following formula (Y-101) to formula (Y-121), constitutional units composed of a divalent heterocyclic ring group represented by the following formula (Y-201) to formula (Y-206), and constitutional units composed of a divalent group in which an arylene group and a divalent heterocyclic ring group are bonded directly represented by the following formula (Y-301) to formula (Y-304).

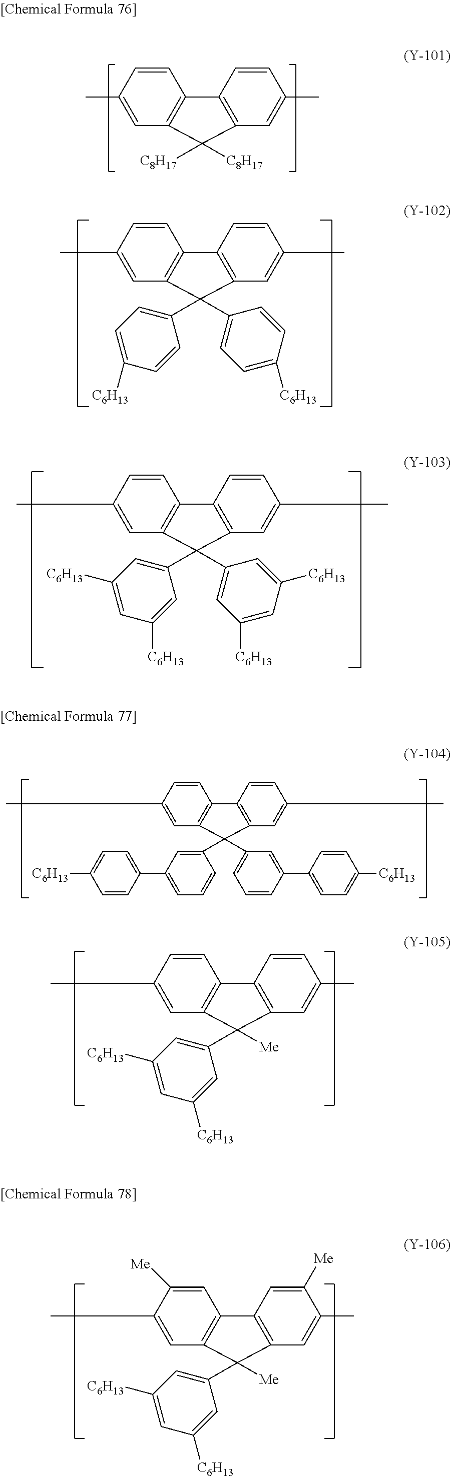

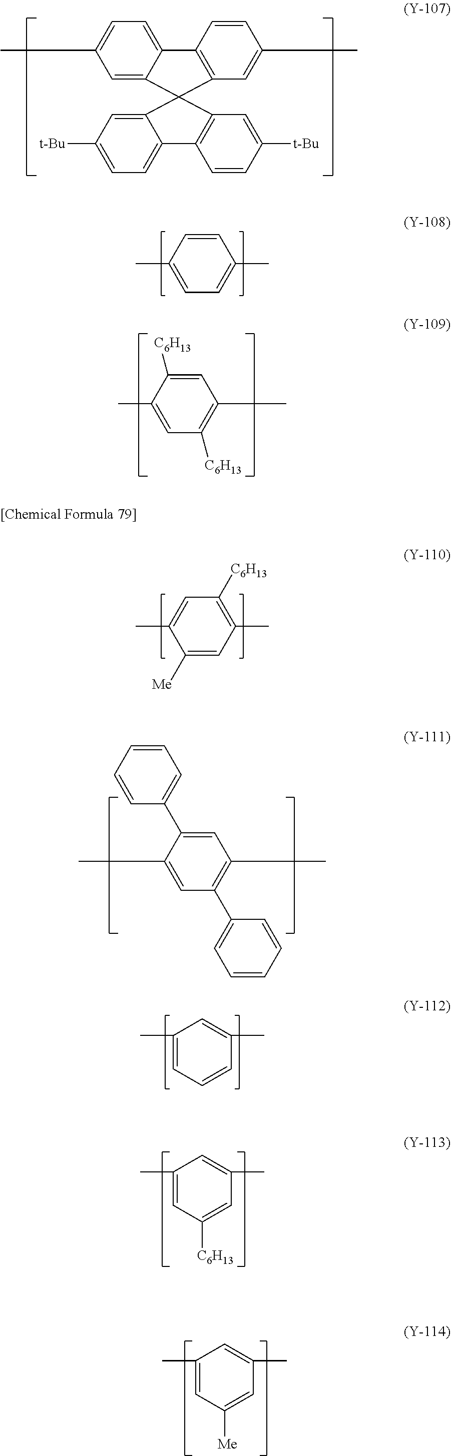

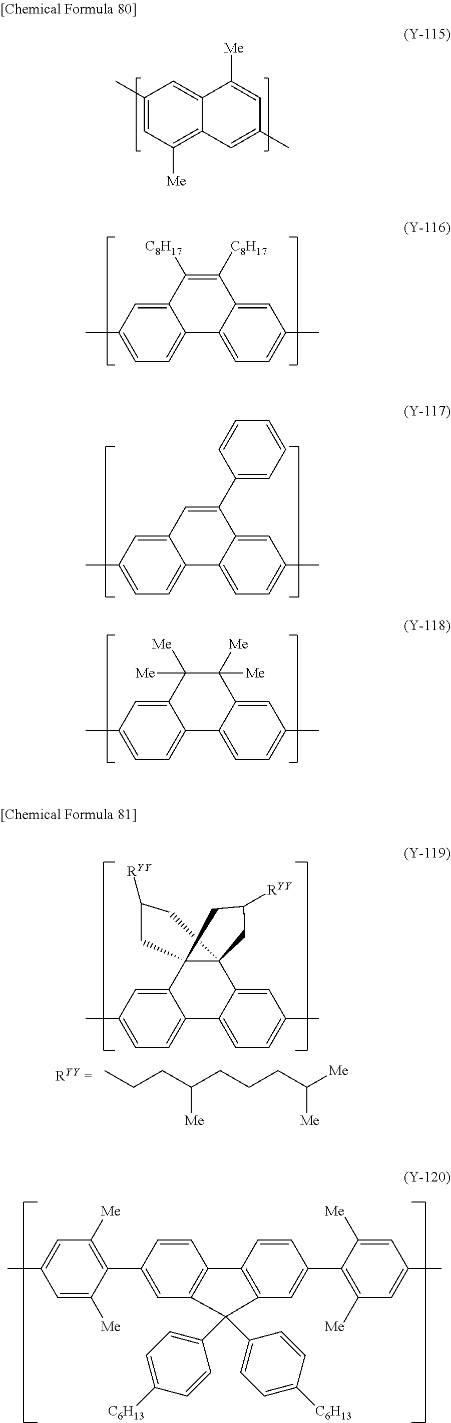

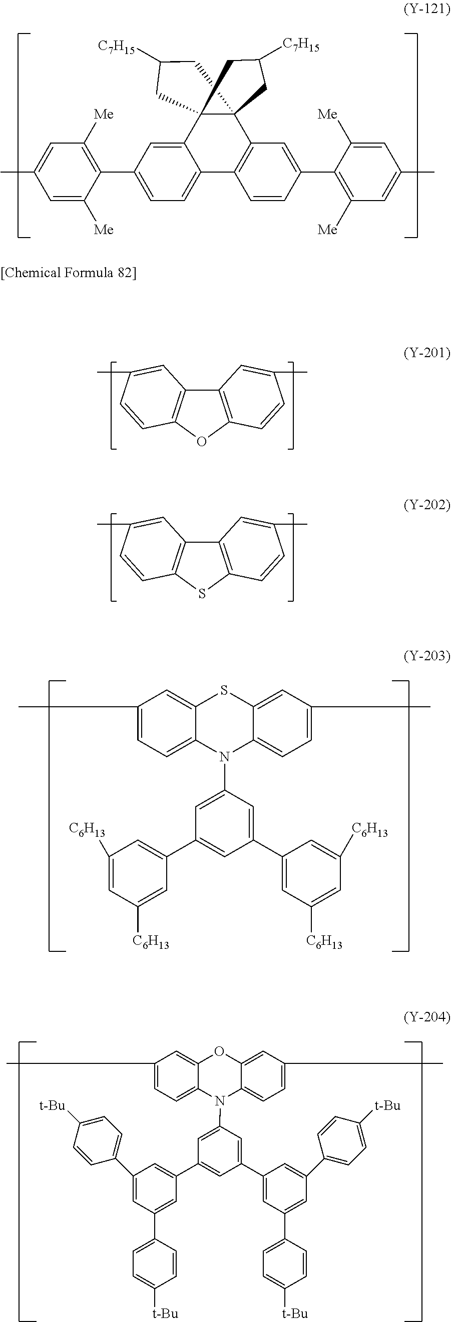

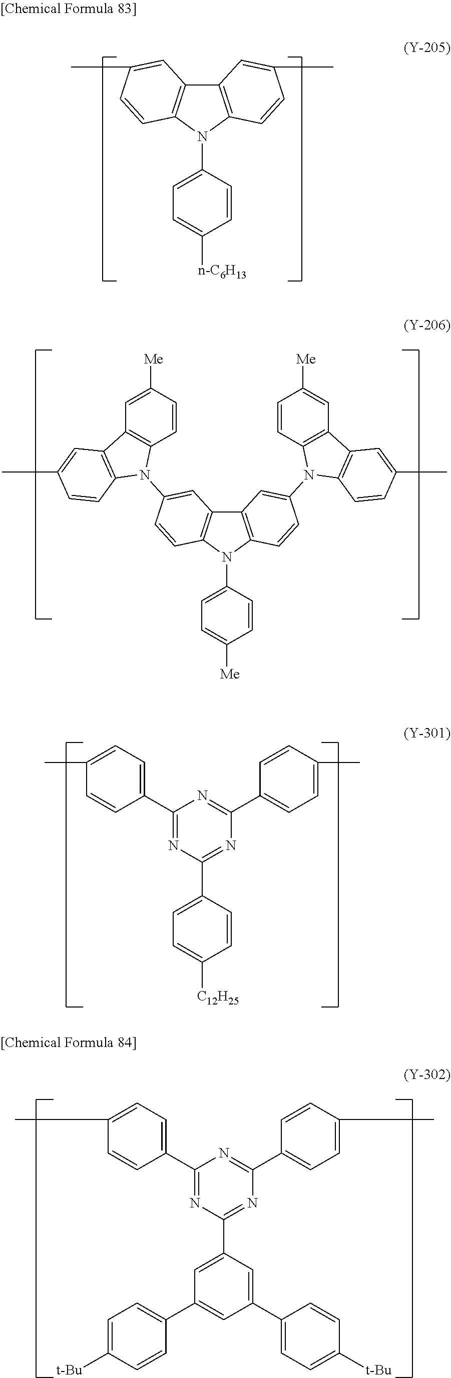

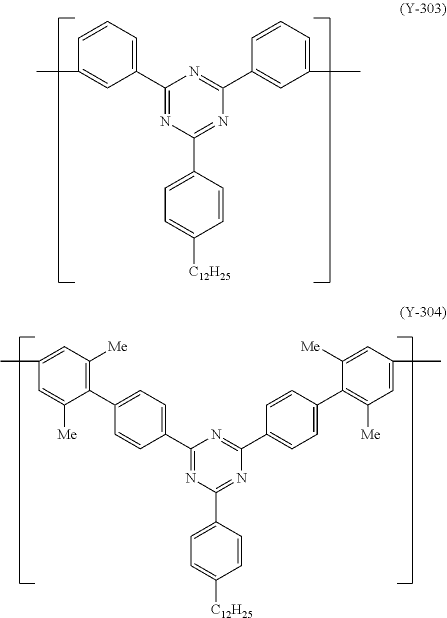

The amount of the constitutional unit represented by the above-described formula (Y) in which Ar^{Y1} is an arylene group is preferably 0.5 to 100% by mol, and more preferably 60 to 95% by mol, with respect to the total amount of constitutional units contained in a polymer compound, since a light emitting device using a composition containing a polymer host and the metal complex of the present embodiment is excellent luminescence lifetime.

The amount of the constitutional unit represented by the above-described formula (Y) in which Ar^{Y1} is a divalent heterocyclic ring group, or a divalent group in which an arylene group and a divalent heterocyclic ring group are bonded directly is preferably 0.5 to 30% by mol, and more preferably 3 to 20% by mol, with respect to the total amount of constitutional units contained in a polymer compound, since a light emitting device using a composition containing a polymer host and the metal complex of the present embodiment is excellent in charge transportability.

The constitutional unit represented by the above-described formula (Y) may be contained only singly or in combination of two or more in a polymer host.

The polymer host preferably further contains a constitutional unit represented by the following formula (X), in addition to the constitutional unit represented by the above-described formula (Y), since its hole transportability is excellent.

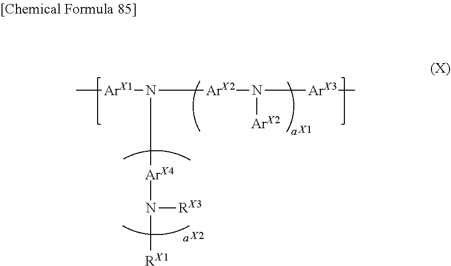

[wherein, a^{X1} and a^{X2} each independently represent an integer of 0 or more; Ar^{X1} and Ar^{X3} each independently represent an arylene group or a divalent heterocyclic ring group, and these groups optionally have a substituent; Ar^{X2} and Ar^{X4} each independently represent an arylene group, a divalent heterocyclic ring group, or a divalent group in which an arylene group and a divalent heterocyclic ring group are bonded directly, and these groups optionally have a substituent; R^{X1}, R^{X2} and R^{X3} each independently represent a hydrogen atom, an alkyl group, a cycloalkyl group, an aryl group or a monovalent heterocyclic ring group, and these groups optionally have a substituent].

a^{X1} is preferably 2 or less, and more preferably 1, since a light emitting device using a composition containing a polymer host and the metal complex of the present embodiment is excellent in luminescence lifetime.

a^{X2} is preferably 2 or less, and more preferably 0, since a light emitting device using a composition containing a polymer host and the metal complex of the present embodiment is excellent in luminescence lifetime.

R^{X1}, R^{X2} and R^{X3} represent preferably an alkyl group, a cycloalkyl group, an aryl group or a monovalent heterocyclic ring group, and more preferably an aryl group, and these groups optionally have a substituent.

The arylene group represented by Ar^{X1} and Ar^{X3} is more preferably a group represented by the above-described formula (A-1) or formula (A-9), and further preferably a group represented by the above-described formula (A-1), and these groups optionally have a substituent.

The divalent heterocyclic ring group represented by Ar^{X1} and Ar^{X3} is more preferably a group represented by the above-described formula (AA-1), formula (AA-2) or formula (AA-7) to formula (AA-26), and these groups optionally have a substituent.

Ar^{X1} and Ar^{X3} represent preferably an arylene group optionally having a substituent.

The arylene group represented by Ar^{X2} and Ar^{X4} is more preferably a group represented by the above-described formula (A-1), formula (A-6), formula (A-7), formula (A-9) to formula (A-11) or formula (A-19), and these groups optionally have a substituent.

The more preferable range of the divalent heterocyclic ring group represented by Ar^{X2} and Ar^{X4} is the same as the more preferable range of the divalent heterocyclic ring group represented by Ar^{X1} and Ar^{X3}.

The more preferable ranges and the further preferable range of an arylene group and a divalent heterocyclic ring group in the divalent group in which an arylene group and a divalent heterocyclic ring group are bonded directly represented by Ar^{X2} and Ar^{X4} are the same as the more preferable ranges and the further preferable ranges of the arylene group and the divalent heterocyclic ring group represented by Ar^{X1} and Ar^{X3}, respectively.

The divalent group in which an arylene group and a divalent heterocyclic ring group are bonded directly represented by Ar^{X2} and Ar^{X4} includes the same groups as the divalent group in which an arylene group and a divalent heterocyclic ring group are bonded directly represented by Ar^{Y1} in the above-described formula (Y).

Ar^{X2} and Ar^{X4} represent preferably an arylene group optionally having a substituent.

The substituent which a group represented by Ar^{X1} to Ar^{X4} and R^{X1} to R^{X3} optionally has is preferably an alkyl group, a cycloalkyl group or an aryl group, and these groups optionally further have a substituent.

The constitutional unit represented by the above-described formula (X) is preferably a constitutional unit represented by the following formula (X-1) to formula (X-7).

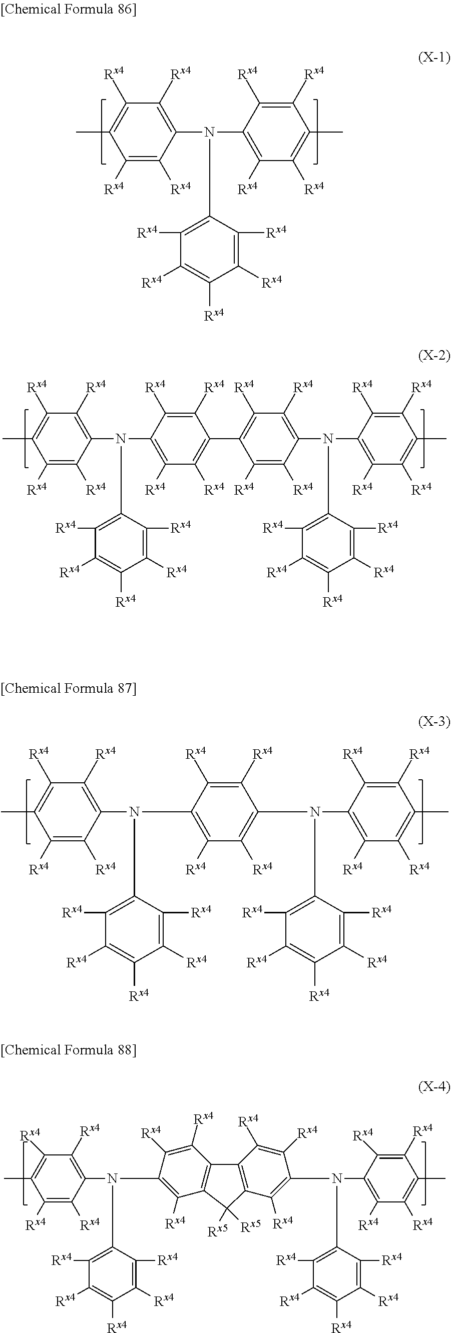

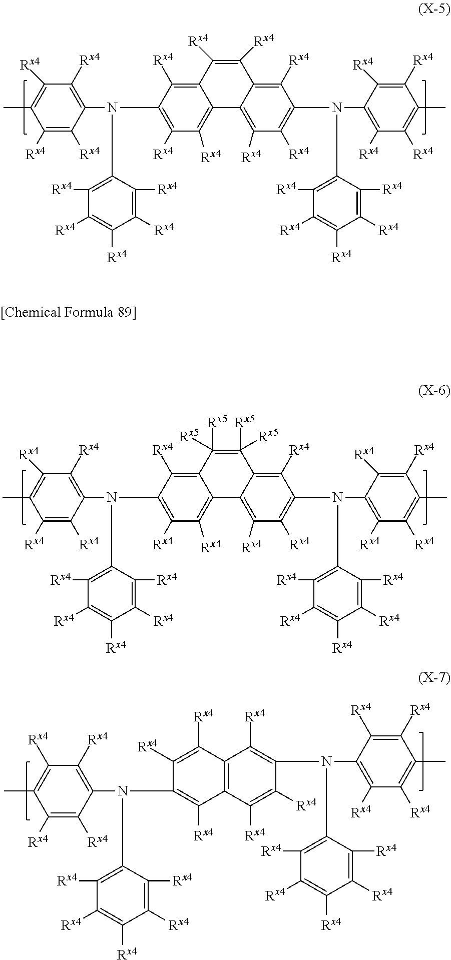

[wherein, R^{X4} and R^{X5} each independently represent a hydrogen atom, an alkyl group, a cycloalkyl group, an alkoxy group, a cycloalkoxy group, an aryl group, an aryloxy group, a halogen atom, a monovalent heterocyclic ring group or a cyano group, and these groups optionally have a substituent; a plurality of R^{X4} may be the same or different; a plurality of R^{X4} may be the same or different, and adjacent groups R^{X5} may be combined together to form a ring together with carbon atoms to which they are attached].

The amount of the constitutional unit represented by the above-described formula (X) is preferably 0.1 to 50% by mol, more preferably 1 to 40% by mol, and further preferably 2 to 30% by mol, with respect to the total amount of constitutional units contained in a polymer host, since its hole transportability is excellent.

The constitutional unit represented by the above-described formula (X) includes, for example, constitutional units represented by the following formula (X1-1) to formula (X1-17).

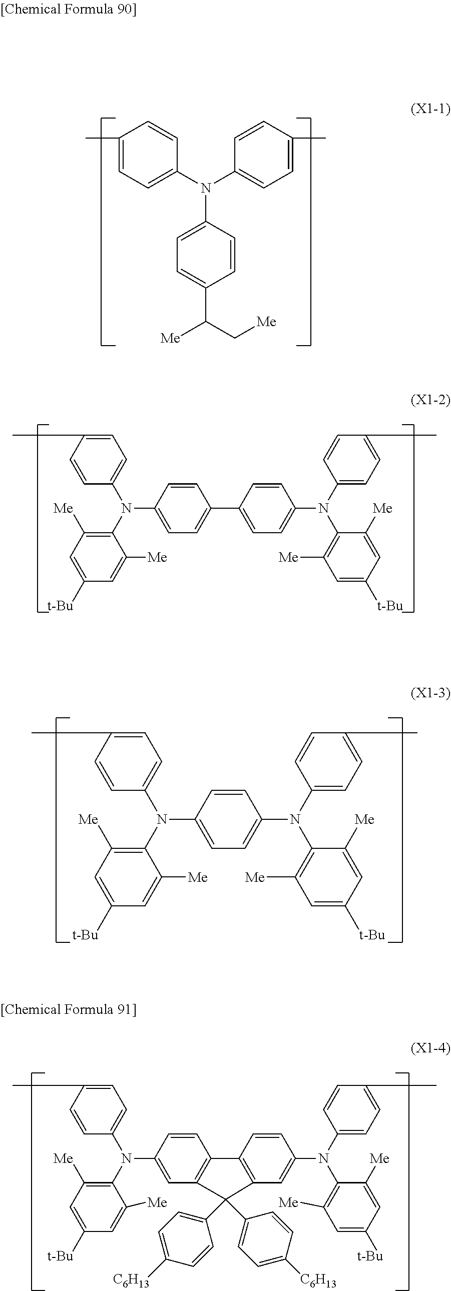

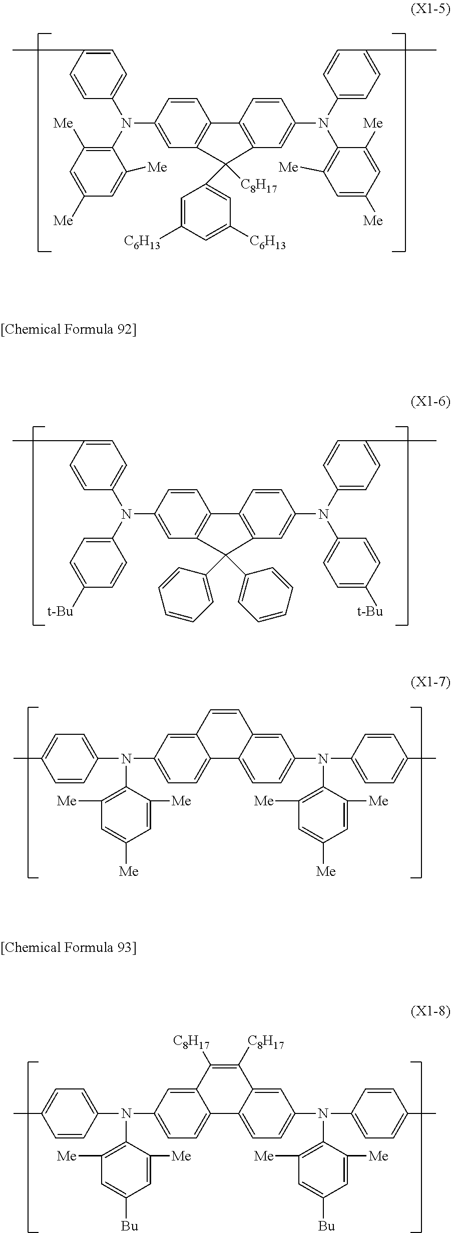

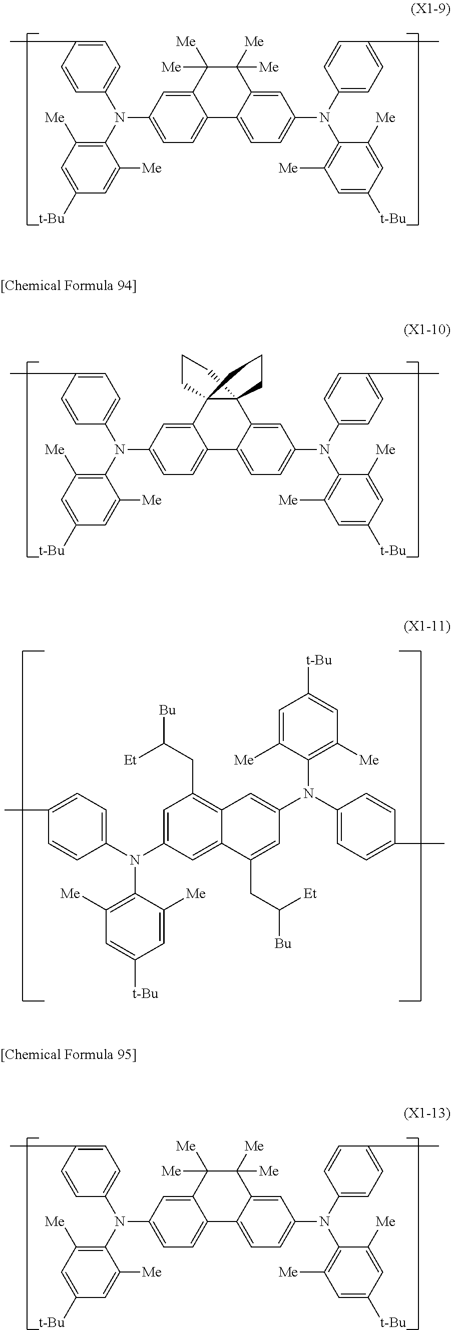

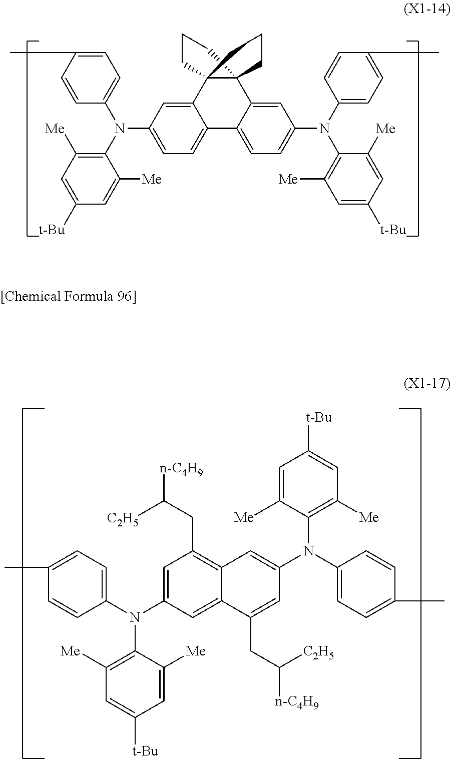

In the polymer host, the constitutional unit represented by the above-described formula (X) may be contained only singly or in combination of two or more.

The polymer host includes, for example, polymer compounds P-1 to P-7 in Table 1. In Table 1, “other” constitutional unit means a constitutional unit represented by the following formula (V), other than the constitutional unit represented by the above-described formula (Y) and the constitutional unit represented by the above-described formula (X).

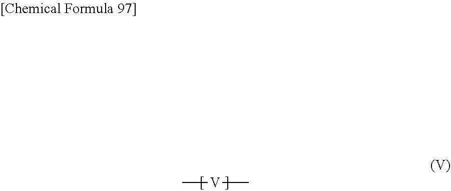

In the formula (V), V represents an oxygen atom, a sulfur atom, a methylene group optionally having a substituent, a group represented by —N(R^{V})— (wherein, R^{V} represents a hydrogen atom, an alkyl group, a cycloalkyl group, an aryl group, or a monovalent heterocyclic ring group, and these groups optionally have a substituent), or a monocyclic or condensed cyclic arylene group optionally having a substituent, and the formula (V) does not correspond to any of the formula (Y) and the formula (X).

In the above-described formula (V), the substituent which V optionally has includes, for example, a hydrogen atom, an alkyl group, a cycloalkyl group, an alkoxy group, a cycloalkoxy group, an aryl group, an aryloxy group, a substituted amino group, and a halogen atom.

TABLE 1

| | Constitutional unit and its molar ratio | | | | |
|---|---|---|---|---|---|
| | | | | Formula | |
| | Formula (Y) | | | (X) | |
| | Formula | Formula | Formula | Formula | |
| | (Y-1) to | (Y-4) to | (Y-8) to | (X-1) to | |
| | formula | formula | formula | formula | |
| Polymer | (Y-2) | (Y-7) | (Y-10) | (X-7) | other |
| compound | p | q | r | s | t |
| P-1 | 0.1~99.9 | 0.1~99.9 | 0 | 0 | 0~30 |
| P-2 | 0.1~99.9 | 0 | 0.1~99.9 | 0 | 0~30 |
| P-3 | 0.1~99.9 | 0 | 0 | 0.1~99.9 | 0~30 |
| P-4 | 0.1~99.8 | 0.1~99.8 | 0 | 0.1~99.8 | 0~30 |
| P-5 | 0.1~99.8 | 0.1~99.8 | 0.1~99.8 | 0 | 0~30 |
| P-6 | 0.1~99.8 | 0 | 0.1~99.8 | 0.1~99.8 | 0~30 |
| P-7 | 0.1~99.7 | 0.1~99.7 | 0.1~99.7 | 0.1~99.7 | 0~30 |

In Table 1, p, q, r, s and t represent the molar ratio of each constitutional unit. p+q+r+s+t=100, and 100≥p+q+r+s≥70. The other constitutional unit means a constitutional unit represented by the above-described formula (V), other than the constitutional unit represented by the above-described formula (Y) and the constitutional unit represented by the above-described formula (X).

The polymer host may be any of a block copolymer, a random copolymer, an alternative copolymer or a graft copolymer, and may also be in other form, and from the above-described standpoint, a copolymer obtained by copolymerizing multiple kinds of raw material monomers is preferred.

The polymer host can be produced using a known polymerization method described in Chemical Reviews (Chem. Rev.), vol. 109, pp. 897-1091 (2009), and the like, and exemplified are methods for polymerizing by a coupling reaction using a transition metal catalyst, such as the Suzuki reaction, the Yamamoto reaction, the Buchwald reaction, the Stille reaction, the Negishi reaction, the Kumada reaction, and the like.

In the above-described polymerization method, the method of charging monomers includes a method of charging the entire amount of monomers into the reaction system at once, a method in which a part of monomers is charged and reacted, and then the remaining monomers are charged in a batch, continuously or dividedly, a method of charging monomers continuously or dividedly, and the like.

The transition metal catalyst includes, for example, a palladium catalyst and a nickel catalyst.

For the post treatment of the polymerization reaction, known methods, for example, a method of removing water-soluble impurities by liquid separation, a method in which the reaction liquid after the polymerization reaction is added to a lower alcohol such as methanol and the like, the deposited precipitate is filtrated, then, dried, and other methods, can be used singly or in combination of two or more. When the purity of the polymer host is low, it can be purified by usual methods such as, for example, recrystallization, reprecipitation, continuous extraction with Soxhlet extractor, column chromatography, and the like.

### [Hole Transporting Material]

The hole transporting material is classified into a low molecular compound and a polymer compound, and a polymer compound is preferable, with a polymer compound having a cross-linkable group being more preferred.

The polymer compound constituting the hole transporting material includes, for example, polyvinylcarbazole and derivatives thereof; and polyarylenes having an aromatic amine structure in the side chain or main chain, and derivatives thereof. Further, the polymer compound constituting the hole transporting material may be a polymer compound containing the constitutional unit represented by the above-described formula (Y), and optionally, the constitutional unit represented by the above-described formula (X), and it is preferable that the polymer compound further has a constitutional unit having a cross-linkable group. The constitutional unit having a cross-linkable group includes, for example, constitutional units represented by the above-described formula (Y) in which an arylene group or a divalent heterocyclic ring group has a cross-linkable group. The polymer compound constituting the hole transporting material may be a compound to which an electron accepting site is bonded. The electron accepting site includes, for example, fullerene, tetrafluorotetracyanoquinodimethane, tetracyanoethylene, trinitrofluorenone, and the like, preferably fullerene.

When the composition of the present embodiment contains a hole transporting material, the blending amount of the hole transporting material is usually 1 part by mass to 400 parts by mass, and preferably 5 parts by mass to 150 parts by mass, with respect to 100 parts by mass of the metal complex of the present embodiment.

The hole transporting material may be used singly or in combination of two or more.

### [Electron Transporting Material]

The electron transporting material is classified into a low molecular compound and a polymer compound. The electron transporting material may have a cross-linkable group.

The low molecular compound constituting the electron transporting material includes, for example, a metal complex having 8-hydroxyquinoline as a ligand, oxadiazole, anthraquinodimethane, benzoquinone, naphthoquinone, anthraquinone, tetracyanoanthraquinodimethane, fluorenone, diphenyldicyanoethylene and diphenoquinone, and derivatives thereof.

The polymer compound constituting the electron transporting material includes, for example, polyphenylene and polyfluorene, and derivatives thereof. The polymer compound may be doped with a metal. Further, the polymer compound constituting the electron transporting material may be a polymer compound containing a constitutional unit represented by the above-described formula (Y), and the polymer compound may further have a constitutional unit having a cross-linkable group. The constitutional unit having a cross-linkable group includes, for example, constitutional units represented by the above-described formula (Y) in which an arylene group or a divalent heterocyclic ring group has a cross-linkable group.

When the composition of the present embodiment contains an electron transporting material, the blending amount of the electron transporting material is usually 1 part by mass to 400 parts by mass, and preferably 5 parts by mass to 150 parts by mass, with respect to 100 parts by mass of the metal complex of the present embodiment.

The electron transporting material may be used singly or in combination of two or more.

### [Hole Injection Material and Electron Injection Material]

The hole injection material and the electron injection material are each classified into a low molecular compound and a polymer compound. The hole injection material and the electron injection material may have a cross-linkable group.

The low molecular compound constituting each of the hole injection material and the electron injection material includes, for example, metal phthalocyanines such as copper phthalocyanine, and the like; carbon; oxides of metals such as molybdenum, tungsten, and the like; metal fluorides such as lithium fluoride, sodium fluoride, cesium fluoride, potassium fluoride, and the like.

The polymer compound constituting each of the hole injection material and the electron injection material includes electrically conductive polymers such as, for example, polyaniline, polythiophene, polypyrrole, polyphenylenevinylene, polythienylenevinylene, polyquinoline and polyquinoxaline, and derivatives thereof; polymers having a group represented by the above-described formula (X) in the main chain or side chain, and the like.

When the composition of the present embodiment contains a hole injection material and/or an electron injection material, the blending amounts of the hole injection material and the electron injection material are usually 1 part by mass to 400 parts by mass, and preferably 5 parts by mass to 150 parts by mass, respectively, with respect to 100 parts by mass of the metal complex of the present embodiment.

The hole injection material and the electron injection material each may be used singly or in combination of two or more.

### [Ion Doping]

When the hole injection material and/or the electron injection material contains a conductive polymer, the electric conductivity of the conductive polymer is preferably 1×10⁻³ S/cm to 1×10³ S/cm. In order to regulate the electric conductivity of the conductive polymer within such a range, the conductive polymer can be doped with an appropriate amount of ions.

The kind of the ion to be doped is an anion for the hole injection material, and a cation for the electron injection material. The anion includes, for example, a polystyrenesulfonate ion, an alkylbenzenesulfonate ion, and a camphorsulfonate ion. The cation includes, for example, a lithium ion, a sodium ion, a potassium ion, and a tetrabutylammonium ion.

The ion to be doped may be used only singly or in combination of two or more.

### [Light Emitting Material]

The light emitting material (different from the metal complex of the present embodiment) is classified into a low molecular compound and a polymer compound. The light emitting material may have a cross-linkable group.

The low molecular compound constituting the light emitting material includes, for example, naphthalene and derivatives thereof, anthracene and derivatives thereof, perylene and derivatives thereof, and, triplet light emitting complexes having iridium, platinum or europium as the central metal.

The polymer compound constituting the light emitting material includes polymer compounds containing, for example, a phenylene group, a naphthalenediyl group, an anthracenediyl group, a fluorenediyldiyl group, a phenanthrenediyl group, a dihydrophenanthrenediyl group, a group represented by the above-described formula (X), a carbazolediyl group, a phenoxazinediyl group, a phenothiazinediyl group, an anthracenediyl group, and/or a pyrenediyl group, and the like.

The light emitting material may contain a low molecular compound and a polymer compound, and preferably contains a triplet light emitting complex and a polymer compound.

The triplet light emitting complex includes, for example, metal complexes shown below.

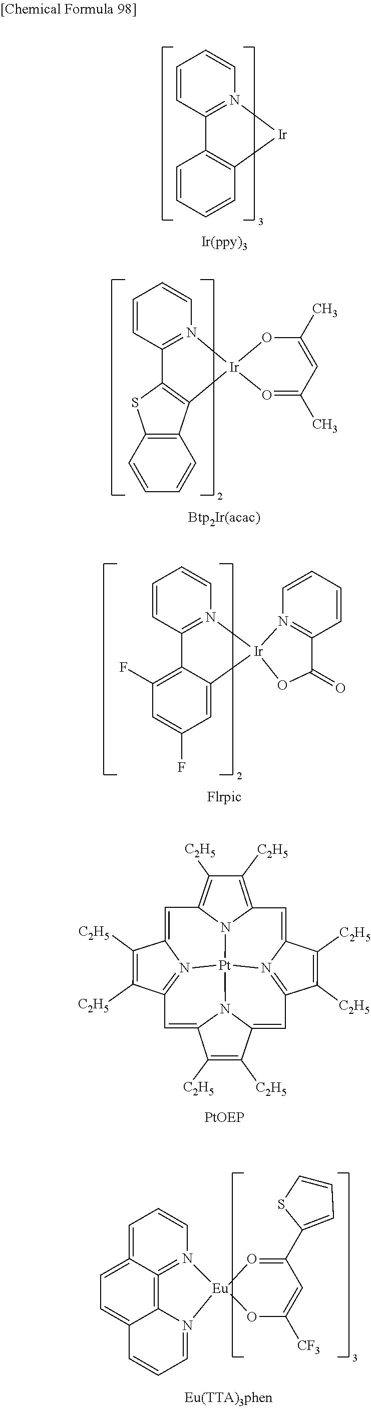

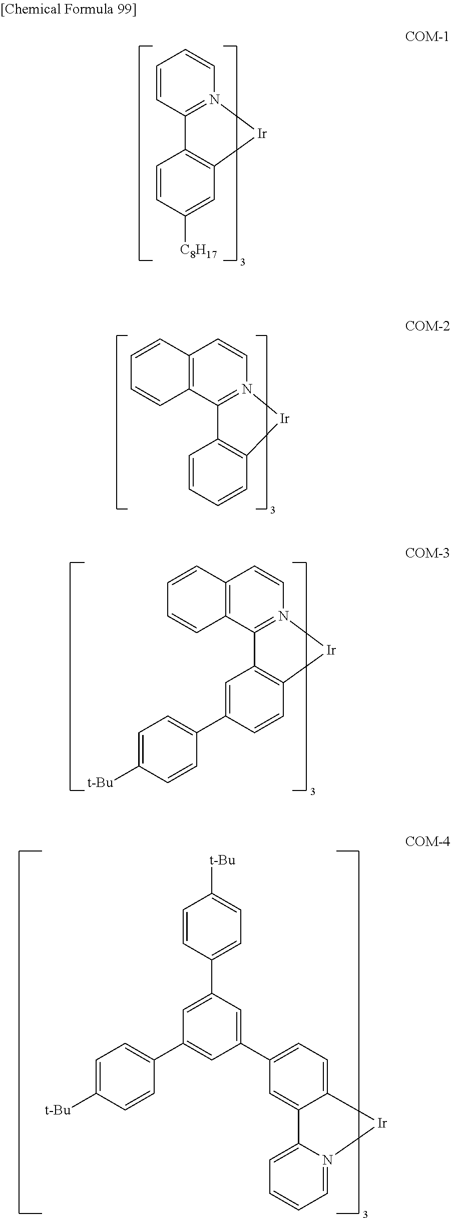

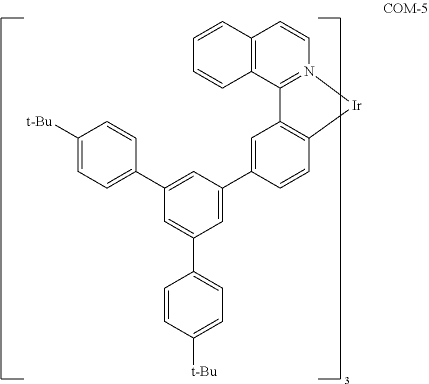

When the composition of the present embodiment contains a light emitting material, the content of the light emitting material is usually 0.1 parts by mass to 400 parts by mass, with respect to 100 parts by mass of the metal complex of the present embodiment.

### [Solvent]

A composition containing the metal complex of the present embodiment and a solvent (hereinafter, referred to as “ink” in some cases) is suitable for fabrication of a light emitting device using a printing method such as an inkjet print method, a nozzle print method, and the like.

The viscosity of the ink may be controlled depending on the type of the printing method, and when applied to printing methods in which a solution passes through a discharge device such as an inkjet print method, and the like, it is preferably 1 to 20 mPa·s at 25° C., for preventing clogging and flight bend in discharging.

The solvent contained in the ink is preferably a solvent capable of dissolving or uniformly dispersing the solid content in the ink. The solvent includes, for example, chlorine-based solvents such as 1,2-dichloroethane, 1,1,2-trichloroethane, chlorobenzene, o-dichlorobenzene, and the like; ether solvents such as tetrahydrofuran, dioxane, anisole, 4-methylanisole, and the like; aromatic hydrocarbon solvents such as toluene, xylene, mesitylene, ethylbenzene, n-hexylbenzene, cyclohexylbenzene, and the like; aliphatic hydrocarbon solvents such as cyclohexane, methylcyclohexane, n-pentane, n-hexane, n-heptane, n-octane, n-nonane, n-decane, n-decane, bicyclohexyl, and the like; ketone solvents such as acetone, methyl ethyl ketone, cyclohexanone, acetophenone, and the like; ester solvents such as ethyl acetate, butyl acetate, ethylcellosolve acetate, methyl benzoate, phenyl acetate, and the like; polyhydric alcohol solvents such as ethylene glycol, glycerin, 1,2-hexanediol, and the like; alcohol solvents such as isopropyl alcohol, cyclohexanol, and the like; sulfoxide solvents such as dimethyl sulfoxide, and the like; and, amide solvents such as N-methyl-2-pyrrolidone, N,N-dimethylformamide, and the like. The solvent may be used singly or in combination of two or more.

In the ink, the blending amount of the solvent is usually 1000 parts by mass to 100000 parts by mass, and preferably 2000 parts by mass to 20000 parts by mass, with respect to 100 parts by mass the metal complex of the present embodiment.

The antioxidant may be a compound not disturbing light emission and charge transportation, and includes, for example, phenolic antioxidants and phosphoric acid-based antioxidants. When the composition contains a solvent, it is preferable that the antioxidant is soluble in the solvent.

When the composition of the present embodiment contains an antioxidant, the blending amount of the antioxidant is usually 0.0001 parts by mass to 10 parts by mass, with respect to 100 parts by mass of the metal complex of the present embodiment.

The antioxidant may be used singly or in combination of two or more.

### <Film>

The film contains the metal complex of the present embodiment.

The film includes also an insolubilized film obtained by insolubilizing the metal complex of the present embodiment in a solvent by crosslinking. The insolubilized film is a film obtained by crosslinking the metal complex of the present embodiment by an external stimulus such as heating (temperature is usually 25 to 300° C.) and/or light irradiation (light is, for example, ultraviolet light, near ultraviolet light, visible light), and the like. Since the insolubilized film is substantially insoluble in a solvent, hence, it can be used for lamination of a light emitting device.

The film is suitable as a hole transporting layer or a hole injection layer in a light emitting device.

The film can be fabricated by, for example, a spin coat method, a casting method, a micro gravure coat method, a gravure coat method, a bar coat method, a roll coat method, a wire bar coat method, a dip coat method, a spray coat method, a screen printing method, a flexo printing method, an offset printing method, an inkjet printing method, a capillary coat method, or a nozzle coat method, using an ink.

The thickness of the film is usually 1 nm to 10 μm.

### <Light Emitting Device>

The light emitting device of the present embodiment is a light emitting device containing the metal complex of the present embodiment.

The constitution of the light emitting device of the present embodiment includes, for example, a constitution containing electrodes consisting of an anode and a cathode, and a layer containing the metal complex of the present embodiment disposed between the electrodes.

### [Layer Constitution]

The layer constituting the light emitting device of the present embodiment is usually at least one layer selected from a light emitting layer, a hole transporting layer, a hole injection layer, an electron transporting layer and an electron injection layer. At least any of layers constituting the light emitting device of the present embodiment contains the metal complex of the present embodiment, and it is preferable that the light emitting layer contains the metal complex of the present embodiment.

The light emitting layer, the hole transporting layer, the hole injection layer, the electron transporting layer and the electron injection layer can be formed by the same method as for the above-described film fabrication using inks prepared by dissolving the materials constituting the layers in the solvent described above, respectively. The materials of the light emitting layer, the hole transporting layer, the hole injection layer, the electron transporting layer and the electron injection layer include the above-described host material, a light emitting material different from the metal complex of the present embodiment, a hole transporting material, a hole injection material, an electron transporting material, and an electron injection material, in addition to the metal complex according to the present embodiment. The light emitting layer can contain, for example, the metal complex of the present embodiment and a host material, and can further contain a light emitting material different from the metal complex of the present embodiment. Further, the hole transporting layer, the hole injection layer, the electron transporting layer and the electron injection layer can contain a hole transporting material, a hole injection material, an electron transporting material and an electron injection material, respectively.

The light emitting device has a light emitting layer between an anode and a cathode. The light emitting device of the present embodiment preferably has at least one of a hole injection layer and a hole transporting layer between an anode and a light emitting layer, from the standpoint of hole injectability and hole transportability, and preferably has at least one of an electron injection layer and an electron transporting layer between a cathode and a light emitting layer, from the standpoint of electron injectability and electron transportability.

When the material of a hole transporting layer, the material of an electron transporting layer and the material of a light emitting layer are soluble in solvents used in forming layers adjacent to the hole transporting layer, the electron transporting layer and the light emitting layer, respectively, in fabrication of a light emitting layer, it is preferable that the materials have a cross-linkable group for avoiding dissolution of the materials in the solvents. By crosslinking the cross-linkable group after forming each layer using the material having the cross-linkable group, the layer can be insolubilized.

The method of forming each of a light emitting layer, a hole transporting layer, an electron transporting layer, a hole injection layer, an electron injection layer and the like in the light emitting device of the present embodiment includes, for example, a vacuum vapor deposition method from a powder and a method by film formation from a solution or melted state when a low molecular compound is used, and, for example, a method by film formation from a solution or melted state when a polymer compound is used.

The order, number and thickness of layers to be laminated may be adjusted in consideration of, for example, external quantum efficiency and device life.

### [Substrate/Electrode]

The substrate in the light emitting device may be a substrate on which electrodes can be formed and which does not chemically change when forming an organic layer, and is a substrate made of a material such as, for example, glass, plastic, silicon, and the like. In the case of an opaque substrate, it is preferable that the electrode farthest from the substrate is transparent or semitransparent.

The anode material includes, for example, electrically conductive metal oxides and semitransparent metals, and preferably includes indium oxide, zinc oxide and tin oxide; electrically conductive compounds such as indium.tin.oxide (ITO), indium.zinc.oxide and the like; argentine-palladium-copper composite (APC); and NESA, gold, platinum, silver, and copper.

The cathode material includes, for example, metals such as lithium, sodium, potassium, rubidium, cesium, beryllium, magnesium, calcium, strontium, barium, aluminum, zinc, and indium, and the like; alloys composed of two or more of them; alloys composed of one or more of metals such as lithium, sodium, potassium, rubidium, cesium, beryllium, magnesium, calcium, strontium, barium, aluminum, zinc, and indium, and the like and one or more of silver, copper, manganese, titanium, cobalt, nickel, tungsten, and tin; and, graphite and graphite intercalation compounds. The alloy includes, for example, a magnesium-silver alloy, a magnesium-indium alloy, a magnesium-aluminum alloy, an indium-silver alloy, a lithium-aluminum alloy, a lithium-magnesium alloy, a lithium-indium alloy, and a calcium-aluminum alloy.

The anode and the cathode each may take a laminated structure composed of two or more layers.

### [Applications]

The light emitting device of the present embodiment is useful for, for example, display and illumination.

Suitable embodiments of the present invention are described above, but the present invention is not limited to the above-described embodiments.

### EXAMPLES

The present invention will be illustrated further in detail by examples below, but the present invention is not limited to these examples.

LC-MS was measured by the following method.

A measurement sample was dissolved in chloroform or tetrahydrofuran so as to give a concentration of about 2 mg/mL, and about 1 μL of the solution was injected into LC-MS (manufactured by Agilent, trade name: 1290 Infinity LC and 6230 TOF LC/MS). As the mobile phase for LC-MS, acetonitrile and tetrahydrofuran were used while changing the ratio thereof, and flowed at a flow rate of 1.0 mL/min. As the column, SUMIPAX ODS Z-CLUE (manufactured by Sumika Chemical Analysis Service, Ltd., internal diameter: 4.6 mm, length: 250 mm, particle diameter: 3 μm) was used.

NMR was measured by the following method.

Five to ten milligrams (5 to 10 mg) of a measurement sample was dissolved in about 0.5 mL of deuterated chloroform (CDCl₃), deuterated tetrahydrofuran, deuterated dimethyl sulfoxide, deuterated acetone, deuterated N,N-dimethylformamide, deuterated toluene, deuterated methanol, deuterated ethanol, deuterated 2-propanol or deuterated methylene chloride, and NMR was measured using an NMR apparatus (trade name: JNM-ECZ400S/L1, manufactured by JEOL RESONANCE, or trade name: AVANCE600, manufactured by Buker).

As the index of the purity of a compound, the value of high performance liquid chromatography (HPLC) area percentage was used. This value was defined as the value at UV=254 nm in HPLC (manufactured by Shimadzu Corp., trade name: LC-20A), unless otherwise stated. In this procedure, a compound to be measured was dissolved in tetrahydrofuran or chloroform so as to give a concentration of 0.01 to 0.2% by mass, and 1 to 10 μL of the solution was injected into HPLC depending on the concentration. As the mobile phase for HPLC, acetonitrile and tetrahydrofuran were used while changing the ratio of acetonitrile/tetrahydrofuran from 100/0 to 0/100 (volume ratio), and flowed at a flow rate of 1.0 mL/min. As the column, SUMIPAX ODS Z-CLUE (manufactured by Sumika Chemical Analysis Service, Ltd., internal diameter: 4.6 mm, length: 250 mm, particle diameter: 3 μm) or an ODS column having the equivalent ability was used. As the detector, a photodiode array detector (manufactured by Shimadzu Corp., trade name: SPD-M20A) was used.

### Example 1

### Synthesis of Metal Complex M1

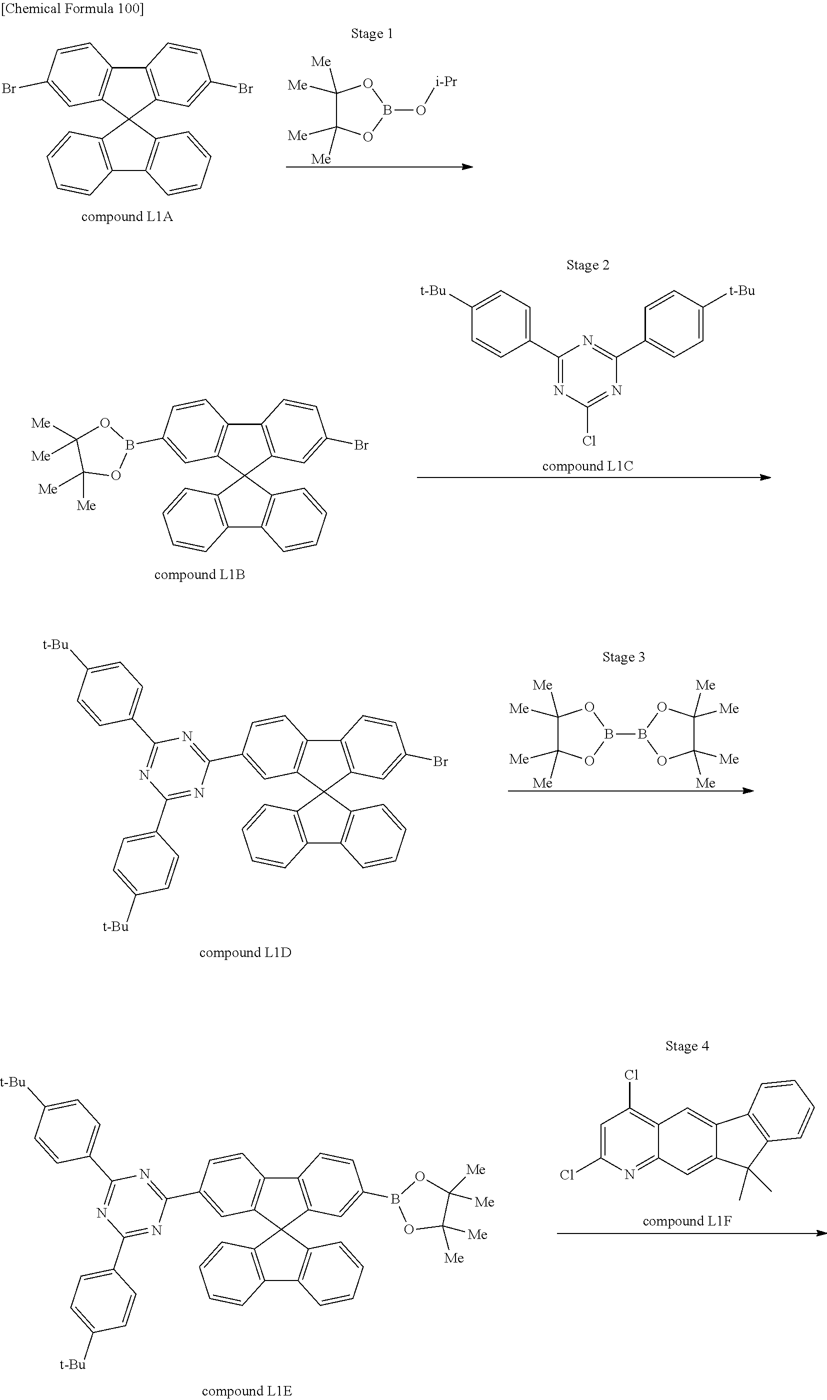

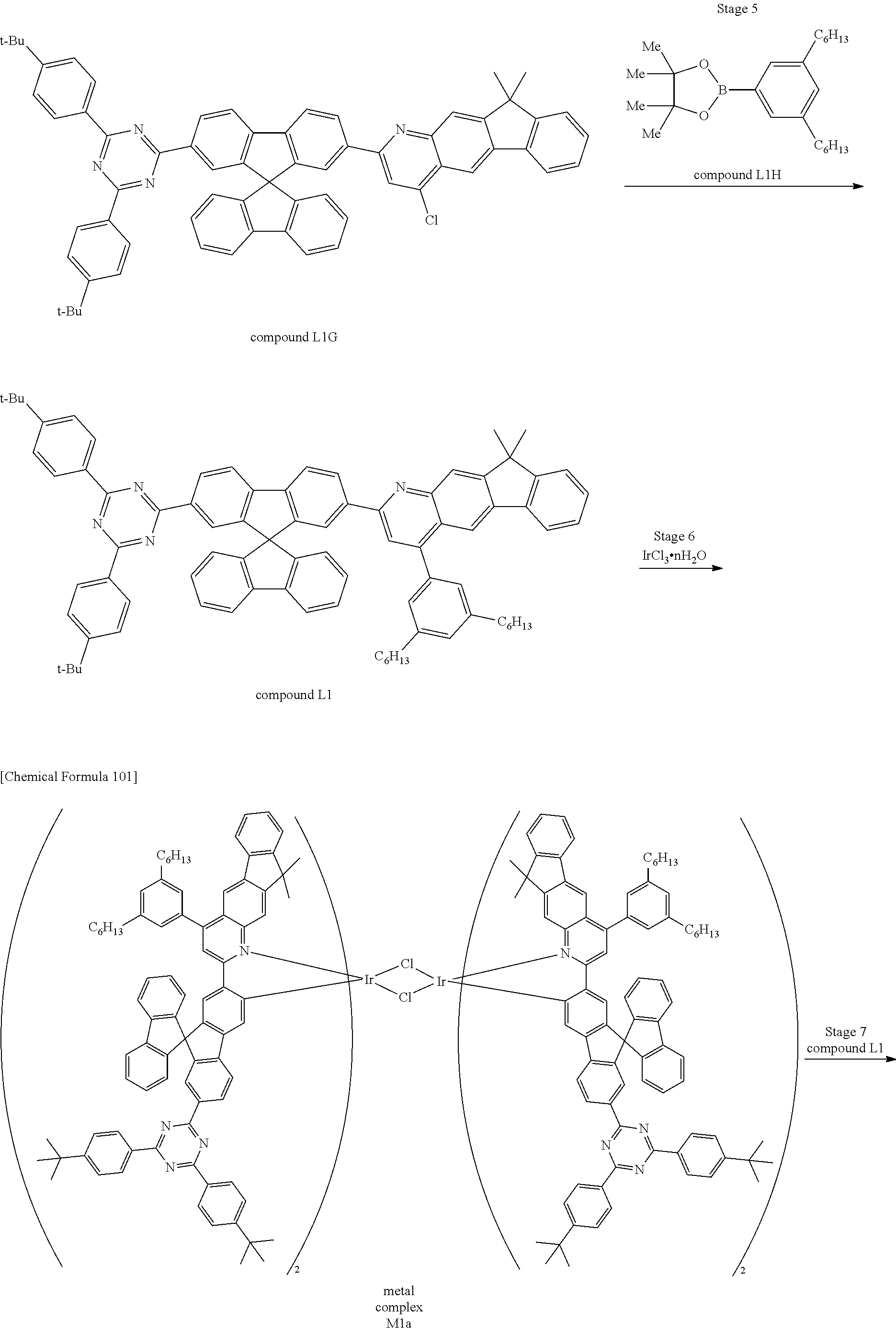

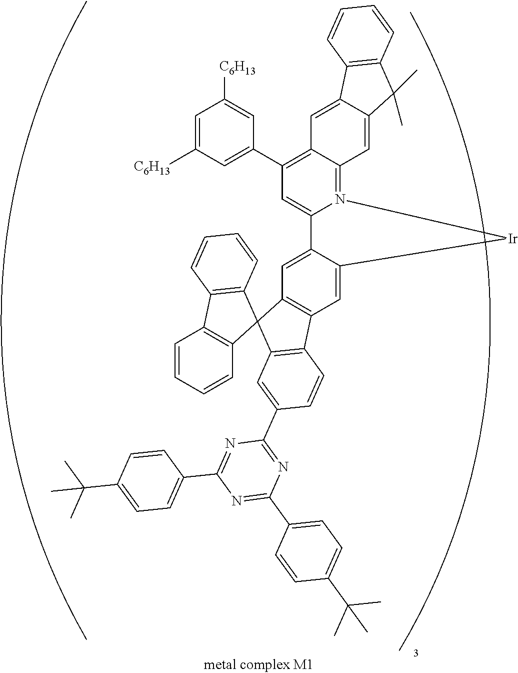

### Synthesis of Compound L1F

A compound L1F was synthesized with reference to a method described in International Publication WO2019/017697.

### Stage 1: Synthesis of Compound L1B

A nitrogen atmosphere was prepared in a reaction vessel, then, a compound L1A (12.2 g) and tetrahydrofuran (600 g) were added, and the mixture was cooled down to −70° C. To this was added a 1.6 M n-butyllithium n-hexane solution (18 mL) slowly, and the mixture was stirred at −70° C. for 1 hour. To this was added isopropoxyboronic acid pinacol ester (7.2 g) slowly, and thereafter, the mixture was stirred at −65° C. for 1 hour. The temperature of the resultant reaction liquid was adjusted to room temperature, then, ion-exchanged water (180 g) and toluene (180 g) were added slowly, and an aqueous layer was removed. The resultant organic layer was washed with ion-exchanged water, and the resultant organic layer was dried over magnesium sulfate, then, filtrated. The resultant filtrate was concentrated under reduced pressure, to obtain a crude product. The resultant crude product was crystallized using toluene and acetonitrile, then, dried under reduced pressure at 50° C., to obtain a compound L1B (8.9 g). The compound L1B had an LC area percentage value of 99.4%.

### Stage 2: Synthesis of Compound L1D

A nitrogen atmosphere was prepared in a reaction vessel, then, the compound L1B (8.0 g), a compound L1C (8.7 g), tetrakis(triphenylphosphine)palladium(0) (1.8 g), a 20% by mass tetraethylammonium hydroxide aqueous solution (50.9 g), ion-exchanged water (50.9 mL), ethanol (80 ml) and toluene (400 mL) were added, and the mixture was stirred at 50° C. for 1 hour. The resultant reaction liquid was cooled down to room temperature, then, and an aqueous layer was removed. The resultant organic layer was washed with ion-exchanged water, then, filtrated, and the resultant filtrate was concentrated under reduced pressure, to obtain a crude product. The resultant crude product was purified by silica gel column chromatography (a mixed solvent of toluene and n-hexane), then, crystallized using toluene and ethanol, and dried under reduced pressure at 50° C., to obtain a compound L1D (7.1 g). The compound L1D had an HPLC area percentage value of 99.2%.

¹H-NMR (400 MHz, CD₂Cl₂) δ (ppm)=7.94-7.93 (m, 3H), 7.82-7.75 (m, 2H), 7.52 (dd, 1H), 7.42 (td, 2H), 7.15 (td, 2H), 7.03 (s, 1H), 6.83 (d, 1H), 6.69 (d, 2H), 1.23 (s, 12H).

### Stage 3: Synthesis of Compound L1E

A nitrogen atmosphere was prepared in a reaction vessel, then, the compound L1D (6.5 g), bis(pinacolato)diboron (2.7 g), dimethoxyethane (130 mL), toluene (130 ml), [1,1′-bis(diphenylphosphino)ferrocene]palladium(II) dichloride.dichloromethane adduct (359 mg) and potassium acetate (1.7 g) were added, and the mixture was stirred at 80° C. for 9 hours. To the resultant reaction liquid was added Celite (6.9 g), then, the liquid was filtrated through a filter paved with silica gel, and the resultant filtrate was concentrated under reduced pressure, to obtain a crude product. To the resultant crude product were added toluene and activated carbon, and the mixture was stirred at room temperature for 30 minutes, then, the liquid was filtrated through a filter paved with Celite, and the resultant filtrate was concentrated under reduced pressure, then, washed with toluene and acetonitrile, and dried under reduced pressure at 50° C., to obtain a compound L1E (6.6 g) as a white solid. The compound L1E had an HPLC area percentage value of 99.5% or more.

¹H-NMR (400 MHz, CD₂Cl₂) δ (ppm)=8.91 (dd, 1H), 8.54 (d, 4H), 8.08 (d, 1H), 8.03 (d, 1H), 7.97 (d, 2H), 7.86 (d, 1H), 7.60-7.52 (m, 5H), 7.46 (t, 2H), 7.18 (t, 2H), 6.86 (d, 1H), 6.79 (d, 2H), 1.38 (s, 18H).

### Stage 4: Synthesis of Compound L1G

A nitrogen atmosphere was prepared in a reaction vessel, then, the compound L1E (6.0 g), the compound L1F (2.6 g), tetrakis(triphenylphosphine)palladium(0) (882 mg), a 20% by mass tetraethylammonium hydroxide aqueous solution (44.6 g), ion-exchanged water (44.6 mL) and toluene (480 mL) were added, and the mixture was stirred at 95° C. for 5 hours. The resultant reaction liquid was cooled down to room temperature, then, ion-exchanged water (120 mL) was added, and an aqueous layer was removed. The resultant organic layer was washed with ion-exchanged water, then, filtrated, and the resultant filtrate was concentrated under reduced pressure, to obtain a crude product. The resultant crude product was crystallized with toluene and ethanol, and dried under reduced pressure at 50° C., to obtain a compound L1G (7.0 g). The compound L1G had an HPLC area percentage value of 99.5% or more.

¹H-NMR (400 MHz, CD₂Cl₂) δ (ppm)=8.91 (d, 1H), 8.54 (d, 4H), 8.13 (d, 1H), 8.05-7.96 (m, 4H), 7.85 (d, 1H), 7.56 (d, 4H), 7.46 (t, 2H), 7.17 (t, 2H), 7.05 (s, 1H), 6.76 (d, 2H), 1.38 (s, 18), 1.25 (s, 12H).

### Stage 5: Synthesis of Compound L1

A nitrogen atmosphere was prepared in a reaction vessel, then, the compound L1G (6.8 g), a compound L1H (3.0 g), tris(dibenzylideneacetone)dipalladium(0) (98.5 mg), 2-dicyclohexylphosphino-2′,6′-dimethoxybiphenyl (150.0 mg), a 40% by mass tetrabutylammonium hydroxide aqueous solution (14.1 g), ion-exchanged water (14.1 mL) and toluene (136 mL) were added, and the mixture was stirred at 80° C. for 1 hour. The resultant reaction liquid was cooled down to room temperature, then, ion-exchanged water (130 mL) was added, and an aqueous layer was removed. The resultant organic layer was washed with ion-exchanged water, then, filtrated, and the resultant filtrate was concentrated under reduced pressure, to obtain a crude product. The resultant crude product was crystallized with toluene and ethanol, and dried under reduced pressure at 50° C., to obtain a compound L1 (8.3 g). The compound L1 had an HPLC area percentage value of 99.5% or more.

¹H-NMR (400 MHz, CD₂Cl₂) δ (ppm)=8.94 (dd, 1H), 8.55 (dd, 4H), 8.35 (dd, 4H), 8.18-8.13 (m, 3H), 8.11 (s, 1H), 8.06-8.02 (m, 3H), 7.72 (s, 1H), 7.70-7.63 (m, 2H), 7.57 (d, 4H), 7.51-7.45 (m, 3H), 7.38-7.27 (m, 2H), 7.26-7.10 (m, 8H), 6.87 (d, 2H), 2.69 (t, 4H), 1.69 (quin, 4H), 1.58 (s, 6H), 1.46-1.30 (m, 30H), 0.90 (t, 6H).

### Stage 6: Synthesis of Metal Complex M1a

An argon gas atmosphere was prepared in a light-shielded reaction vessel, then, iridium(III) chloride hydrate (390 mg), the compound L1 (2.80 g) and 2-ethoxyethanol (12 mL) were added, and the mixture was stirred at 140° C. for 24 hours.

The resultant reaction mixture was cooled down to room temperature, then, added into methanol (20 mL), and the mixture was stirred at room temperature for 1 hour. Thereafter, filtration was performed, and the resultant residue was dried under reduced pressure, to obtain a red solid (1.98 g) containing a metal complex M1a.

### Stage 7: Synthesis of Metal Complex M1

An argon gas atmosphere was prepared in a light-shielded reaction vessel, then, the red solid (1.98 g) containing a metal complex M1a, the compound L1 (946.5 mg), silver(I) trifluoromethanesulfonate (272.4 mg), 2,6-lutidine (124 μL) and a diethylene glycol dimethyl ether (20 mL) were added, and the mixture was stirred at 157° C. for 72 hours.

The resultant reaction mixture was cooled down to room temperature, then, added into methanol (60 mL), and the mixture was stirred at room temperature for 1 hour. Thereafter, filtration was performed, to the resultant residue was added toluene (50 mL), the liquid was filtrated through a filter paved with Celite, and the resultant filtrate was concentrated under reduced pressure, to obtain a solid.

The resultant solid was purified by silica gel column chromatography (toluene/hexane) and crystallization (toluene/ethanol) in sequence, then, dried under reduced pressure, to obtain a metal complex M1 (705 mg, yield 24% with respect to the charged amount of IrCl₃.3H₂O wherein iridium(III) chloride hydrate is a trihydrate) as a red solid. The metal complex M1 showed an HPLC area percentage value of 99.4%.

LC-MS (APCI positive): m/z=3632.9[M]⁺

¹H-NMR (400 MHz, CD₂Cl₂) δ (ppm)=8.85-8.78 (m, 6H), 8.53 (d, 12H), 8.20 (s, 3H), 8.09 (s, 3H), 8.02-7.95 (m, 6H), 7.91 (s, 3H), 7.72-7.63 (m, 6H), 7.62-7.45 (m, 21H), 7.41 (t, 3H), 7.30-7.11 (m, 21H), 7.09 (t, 3H), 7.02 (d, 3H), 6.86 (d, 3H), 2.66 (t, 12H), 1.65 (t, 12H), 1.45 (m, 90H), 0.85 (s, 18H), 0.51 (s, 9H), 0.16 (s, 9H).

### Example 2

### Synthesis of Metal Complex M2

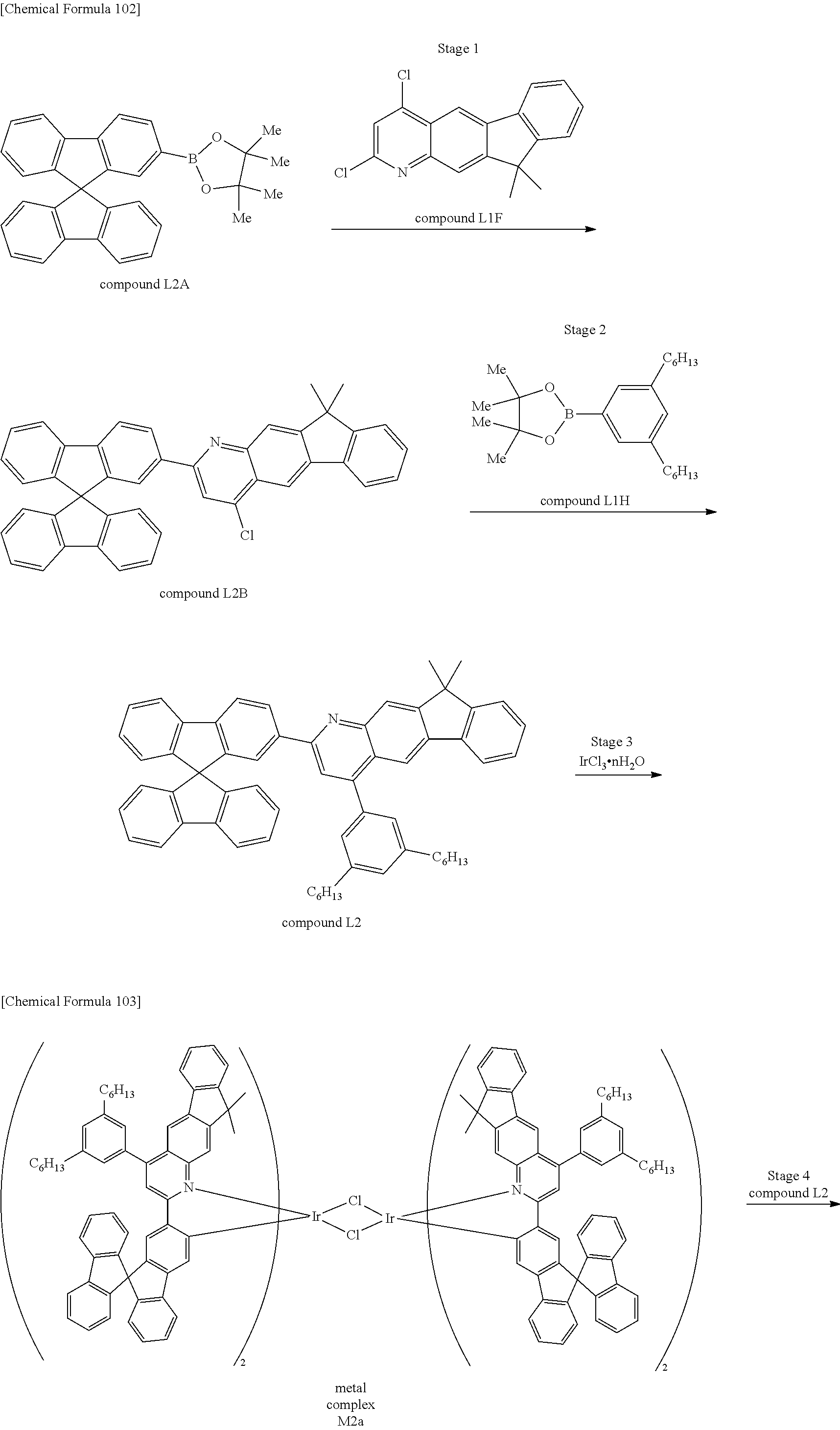

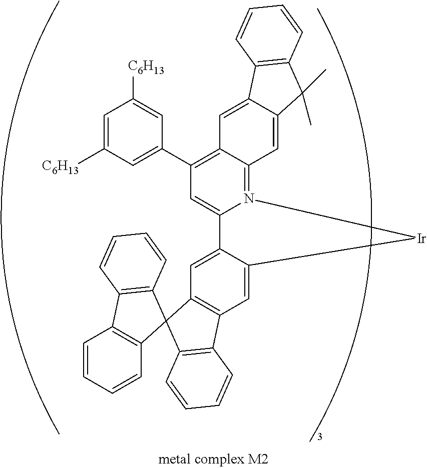

### Stage 1: Synthesis of Compound L2B

A nitrogen atmosphere was prepared in a reaction vessel, then, a compound L2A (13.9 g), the compound L1F (9.0 g), tetrakis(triphenylphosphine)palladium(0) (3.3 g), 20% by mass tetraethylammonium hydroxide aqueous solution (84.2 g) and toluene (180 mL) were added, and the mixture was stirred at 65° C. for 2 hours. The resultant reaction liquid was cooled down to room temperature, then, the solid was filtrated, and washed with ion-exchanged water, ethanol and toluene. The resultant solid was dissolved in chloroform, and the solution was filtrated through a filter paved with Celite and silica gel, and the resultant filtrate was concentrated under reduced pressure, to obtain a solid. The resultant crude product was crystallized with toluene and ethanol, and dried under reduced pressure at 50° C., to obtain a compound L2B (14.1 g). The compound L2B had an HPLC area percentage value of 99.5% or more.

### Stage 2: Synthesis of Compound L2

A nitrogen atmosphere was prepared in a reaction vessel, then, the compound L2B (13.0 g), the compound L1H (8.96 g), tris(dibenzylideneacetone)dipalladium(0) (307 mg), 2-dicyclohexylphosphino-2′,6′-dimethoxybiphenyl (453 mg), a 40% by mass tetrabutylammonium hydroxide aqueous solution (42.7 g), ion-exchanged water (42.7 mL) and toluene (260 mL) were added, and the mixture was stirred at 60° C. for 4 hours. The resultant reaction liquid was cooled down to room temperature, then, ion-exchanged water (130 mL) was added, and an aqueous layer was removed. The resultant organic layer was washed with ion-exchanged water, then, filtrated, and the resultant filtrate was concentrated under reduced pressure, to obtain a crude product. The resultant crude product was crystallized with toluene and ethanol, and dried under reduced pressure at 50° C., to obtain a compound L2 (15.6 g). The compound L2 had an HPLC area percentage value of 99.5% or more.

¹H-NMR (400 MHz, CDCl₃) δ (ppm)=8.33 (dd, 1H), 8.12 (s, 1H), 8.09 (s, 1H), 8.01 (d, 1H), 7.93-7.85 (m, 3H), 7.65 (d, 1H), 7.58 (s, 1H), 7.53 (d, 1H), 7.45 (d, 1H), 7.42-7.26 (m, 5H), 7.16-7.08 (m, 6H), 6.79 (d, 2H), 6.72 (d, 1H), 2.67 (t, 4H), 1.67 (quin, 4H), 1.58 (s, 6H), 1.44-1.26 (m, 12H), 0.89 (t, 6H).

### Stage 3: Synthesis of Metal Complex M2a

An argon gas atmosphere was prepared in a light-shielded reaction vessel, then, iridium(III) chloride hydrate (1.5 g), the compound L2 (7.2 g) and 2-ethoxyethanol (45 mL) were added, and the mixture was stirred at 130° C. for 24 hours.

The resultant reaction mixture was cooled down to room temperature, then, added into methanol (75 g), and the mixture was stirred at room temperature for 1 hour. Thereafter, filtration was performed, and the resultant residue was dried under reduced pressure, to obtain a red solid (7.5 g) containing a metal complex M2a.

### Stage 4: Synthesis of Metal Complex M2

An argon gas atmosphere was prepared in a light-shielded reaction vessel, then, the red solid (7.40 g) containing a metal complex M2a, the compound L2 (3.41 g), silver(I) trifluoromethanesulfonate (1.40 g), 2,6-lutidine (628 μL) and diethylene glycol dimethyl ether (74 mL) were added, and the mixture was stirred at 150° C. for 72 hours.

The resultant reaction mixture was cooled down to room temperature, then, added into methanol (100 mL), and the mixture was stirred at room temperature for 1 hour. Thereafter, filtration was performed, to the resultant residue was added toluene (120 mL), the liquid was filtrated through a filter paved with Celite, and the resultant filtrate was concentrated under reduced pressure, to obtain a solid.

The resultant solid was purified by silica gel column chromatography (toluene/hexane) and crystallization (toluene/heptane) in sequence, then, dried under reduced pressure, to obtain a metal complex M2 (3.0, yield 29% with respect to the charged amount of IrCl₃.3H₂O wherein iridium(III) chloride hydrate is a trihydrate) as a red solid. The metal complex M2 showed an HPLC area percentage value of 99.5% or more.

¹H-NMR (400 MHz, CD₂Cl₂) δ (ppm)=8.76 (s, 3H), 8.16 (s, 3H), 7.93-7.84 (m, 9H), 7.58 (d, 3H), 7.50-7.42 (m, 6H), 7.39-7.29 (m, 6H), 7.28-7.08 (m, 24H), 7.06-6.97 (m, 12H), 6.74 (d, 3H), 6.54 (d, 3H), 2.64 (t, 12H), 1.63 (quin, 12H), 1.40-1.20 (m, 36H), 0.88 (t, 18H), 0.48 (s, 9H), 0.13 (s, 9H).

### Comparative Example 1,2

### <Acquisition of Metal Complexes CM1 and CM2>

Metal complexes CM1 and CM2 manufactured by Luminescense Technology were used.

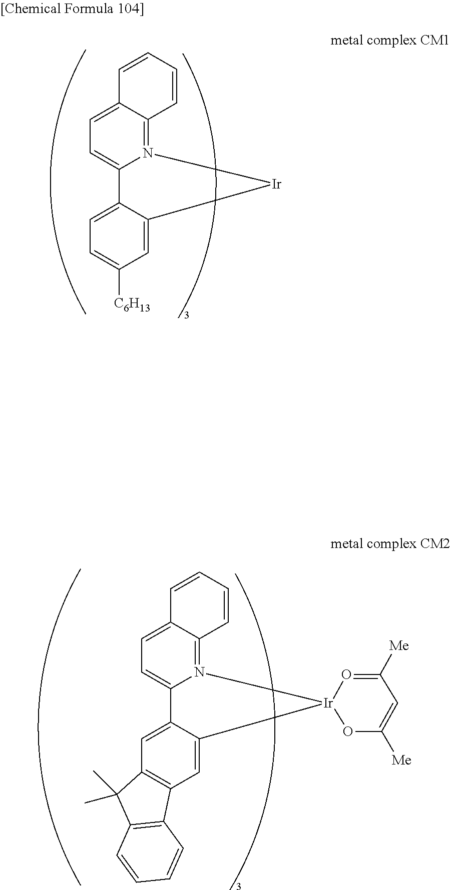

### Comparative Example 3

### Synthesis of Metal Complex CM3

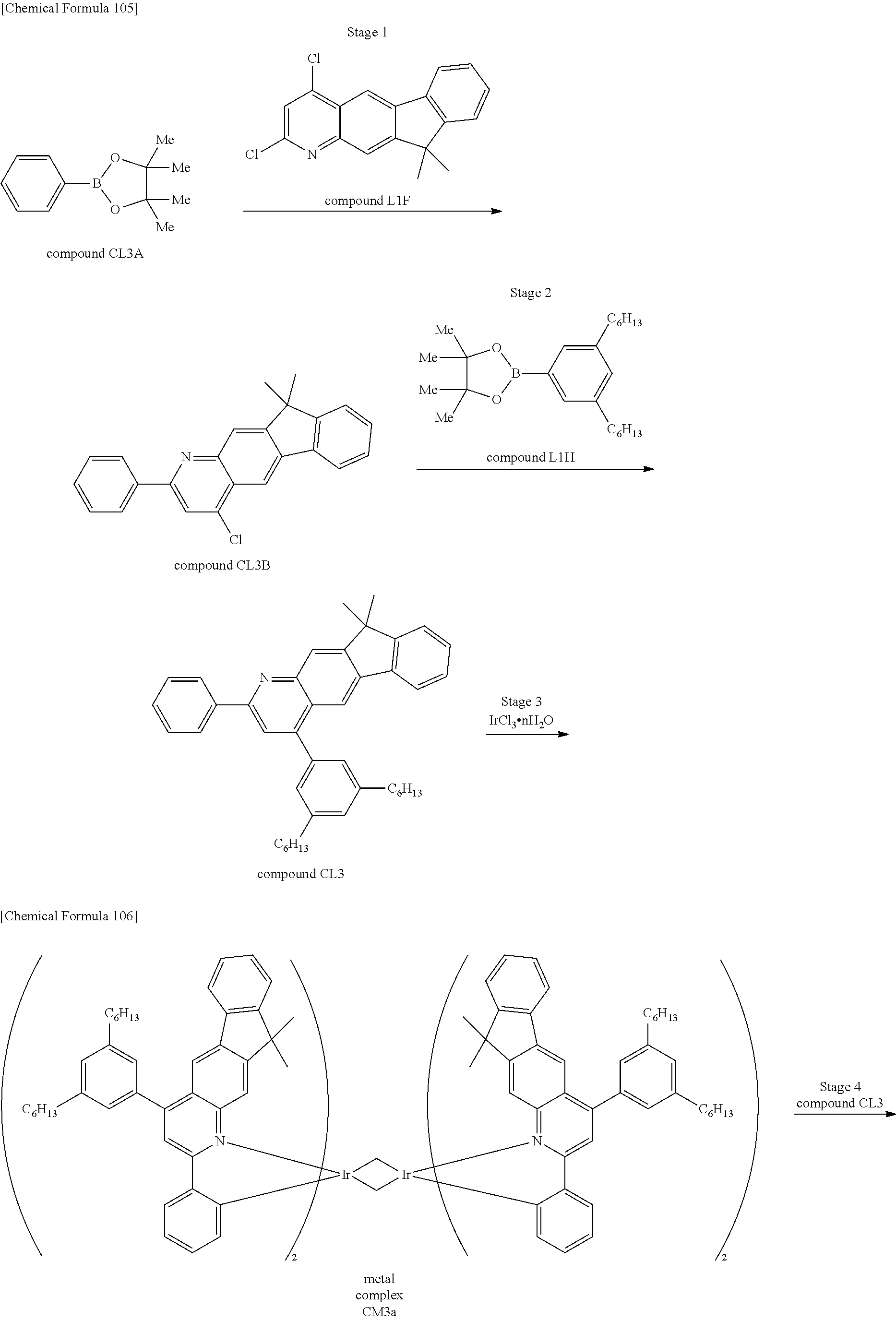

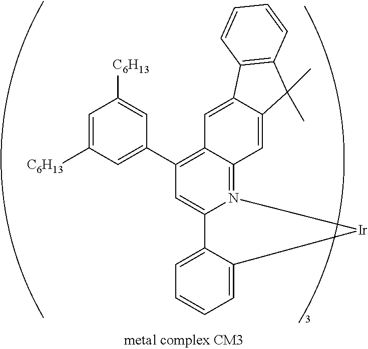

### Stage 1: Synthesis of Compound CL3B

A nitrogen atmosphere was prepared in a reaction vessel, then, a compound CL3A (6.4 g), the compound L1F (9.0 g), tetrakis(triphenylphosphine)palladium(0) (3.3 g), a 20% by mass tetraethylammonium hydroxide aqueous solution (48.2 g), ion-exchanged water (36 g) and toluene (180 g) were added, and the mixture was stirred at 70° C. for 3 hours. The resultant reaction liquid was cooled down to room temperature, then, ion-exchanged water (90 g) was added, and an aqueous layer was removed. The resultant organic layer was washed with ion-exchanged water, then, filtrated, and the resultant filtrate was concentrated under reduced pressure, to obtain crude product. The resultant crude product was dissolved in toluene, and the solution was filtrated through a filter paved with silica gel, and the resultant filtrate was concentrated under reduced pressure, to obtain a solid. The resultant solid was crystallized with toluene and ethanol, and dried under reduced pressure at 50° C., to obtain a compound CL3B (8.2 g). The compound CL3B had an HPLC area percentage value of 99.3%. This step was repeated until a necessary amount of the compound CL3B was obtained.

### Stage 2: Synthesis of Compound CL3

A nitrogen atmosphere was prepared in a reaction vessel, then, the compound CL3B (8.8 g), the compound L1H (10.1 g), tris(dibenzylideneacetone)dipalladium(0) (347 mg), 2-dicyclohexylphosphino-2′,6′-dimethoxybiphenyl (512 mg), a 40% by mass tetrabutylammonium hydroxide aqueous solution (48.1 g), ion-exchanged water (48.1 g) and toluene (88.0 g) were added, and the mixture was stirred at 70° C. for 2 hours. The resultant reaction liquid was cooled down to room temperature, then, ion-exchanged water (88.0 g) was added, and an aqueous layer was removed. The resultant organic layer was washed with ion-exchanged water, then, filtrated, and the resultant filtrate was concentrated under reduced pressure, to obtain a crude product. The resultant crude product was purified by silica gel column chromatography (toluene/hexane), and dried under reduced pressure at 50° C., to obtain a compound CL3 (12.6 g). The compound CL3 had an HPLC area percentage value of 99.3% or more.

¹H-NMR (400 MHz, CDCl₃) δ (ppm)=8.26-8.20 (m, 4H), 7.81 (s, 1H), 7.71 (d, 1H), 7.56-7.44 (m, 4H), 7.39-7.30 (m, 2H), 7.30-7.35 (m, 2H), 7.18 (s, 1H), 2.72 (t, 4H), 1.73 (quin, 4H), 1.63 (s, 6H), 1.49-1.25 (m, 12H), 0.90 (t, 6H).

### Stage 3: Synthesis of Metal Complex CM3a

An argon gas atmosphere was prepared in a light-shielded reaction vessel, then, iridium(III) chloride hydrate (1.46 g), the compound CL3 (5.19 g) and 2-ethoxyethanol (44 mL) were added, and the mixture was stirred at 140° C. for 24 hours.

The resultant reaction mixture was cooled down to room temperature, then, added into methanol (73 mL), and the mixture was stirred at room temperature for 1 hour. Thereafter, filtration was performed, and the resultant residue was dried under reduced pressure, to obtain a red solid (3.67 g) containing a metal complex CM3a.

### Stage 4: Synthesis of Metal Complex CM3

An argon gas atmosphere was prepared in a light-shielded reaction vessel, then, the red solid (1.98 g) containing a metal complex CM3a, the compound CL3 (946.5 mg), silver(I) trifluoromethanesulfonate (272.4 mg), 2,6-lutidine (124 μL) and a diethylene glycol dimethyl ether (20 mL) were added, and the mixture was stirred at 150° C. for 48 hours.

The resultant reaction mixture was cooled down to room temperature, then, added into methanol (60 mL), and the mixture was stirred at room temperature for 1 hour. Thereafter, filtration was performed, to the resultant residue was added toluene (50 mL), the liquid was filtrated through a filter paved with Celite, and the resultant filtrate was concentrated under reduced pressure, to obtain a solid.

The resultant solid was purified by silica gel column chromatography (toluene/hexane) and crystallization (toluene/heptane) in sequence, then, dried under reduced pressure, to obtain a metal complex CM3 (1.15 g, yield 16% with respect to the charged amount of IrCl₃.3H₂O wherein iridium(III) chloride hydrate is a trihydrate) as a red solid. The metal complex CM3 showed an HPLC area percentage value of 99.0%.

¹H-NMR (400 MHz, CD₂Cl₂) δ (ppm)=8.75 (s, 3H), 8.27 (s, 3H), 8.17 (s, 3H), 7.89 (d, 3H), 7.63 (d, 3H), 7.30-7.13 (m, 15H), 7.07 (d, 3H), 6.89 (t, 3H), 6.72 (t, 3H), 6.52 (d, 3H), 2.74 (t, 12H), 1.72 (quin, 12H), 1.46-1.22 (m, 36H), 0.88 (t, 18H), 0.53 (s, 9H), 0.22 (s, 9H).

### Synthesis Example 1

### Synthesis of Polymer Compound IP1

A polymer compound IP1 was synthesized by a method described in JP-A No. 2012-144722, using a monomer PM1 synthesized according to a method described in JP-A No. 2011-174062, a monomer PM2 synthesized according to a method described in International Publication WO2005/049546, a monomer PM3 synthesized according to a method described in International Publication WO2002/045184, and a monomer PM4 synthesized according to a method described in JP-A No. 2008-106241.

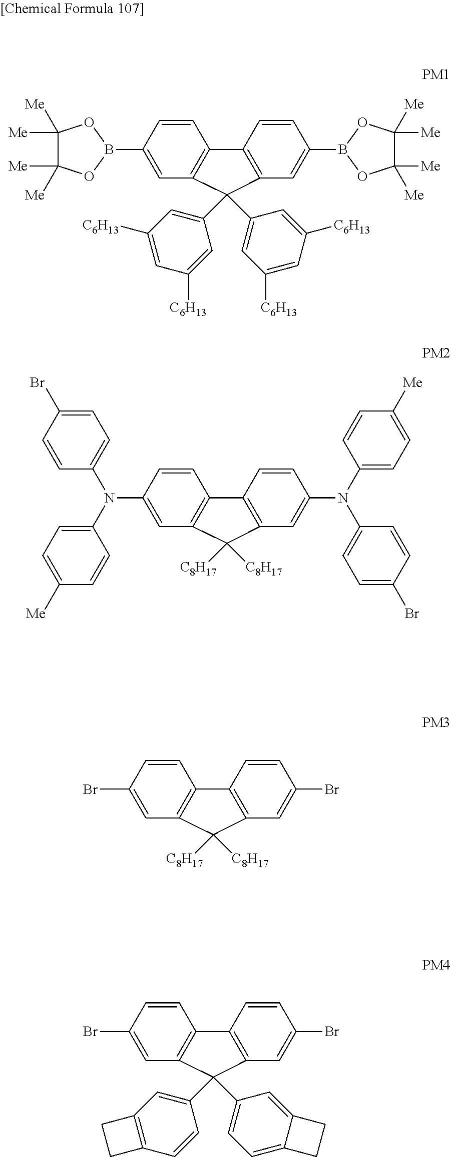

The polymer compound IP1 is a copolymer constituted of a constitutional unit derived from the monomer PM1, a constitutional unit derived from the monomer PM2, a constitutional unit derived from the monomer PM3, and a constitutional unit derived from the monomer PM4 at a molar ratio of 50:30:12.5:7.5, according to the theoretical values calculated from the amounts of the charging raw materials.

### Synthesis Example 2

### Synthesis of Polymer Compound P1

A polymer compound P1 was synthesized by a method described in International Publication WO2012/133381, using the above-described monomer PM3, a monomer PM5 synthesized according to a method described in International Publication WO2005/056633, and a monomer PM6 synthesized according to a method described in International Publication WO2011/078391.

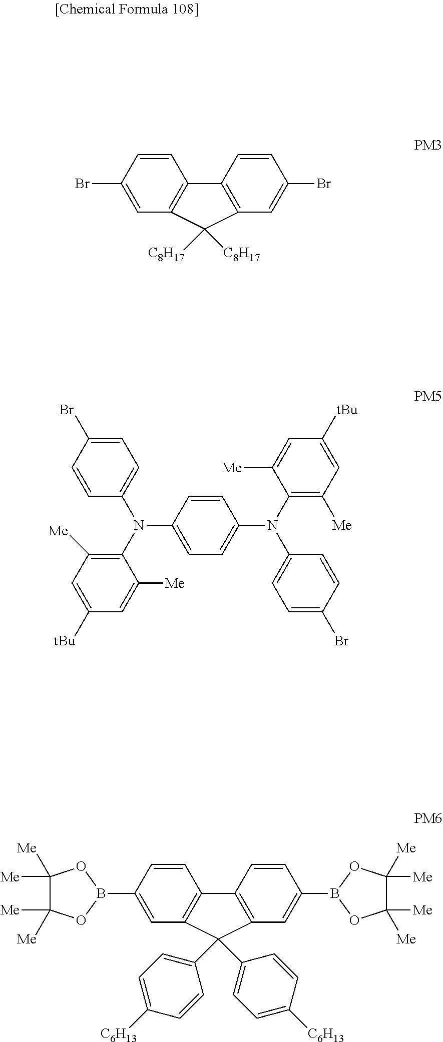

The polymer compound P1 is a copolymer constituted of a constitutional unit derived from the monomer PM3, a constitutional unit derived from the monomer PM5, and a constitutional unit derived from the monomer PM6 at a molar ratio of 50:5:45, according to the theoretical values calculated from the amounts of the charging raw materials.

### Example D1

### <Fabrication of Light Emitting Device D1>

### (Formation of Anode and Hole Injection Layer)

An ITO film was attached with a thickness of 45 nm on a glass substrate by a sputtering method, to form an anode. On the anode, a hole injection material ND-3202 (manufactured by Nissan Chemical Corporation) was spin-coated to form a film with a thickness of 65 nm. The substrate carrying the hole injection layer laminated thereon was placed under an air atmosphere, and heated on a hot plate at 50° C. for 3 minutes, and further, heated at 240° C. for 15 minutes, to form a hole injection layer.

### (Formation of Hole Transporting Layer)

The polymer compound IP1 as a hole transporting material was dissolved at a concentration of 0.6% by mass in xylene as a solvent. The resultant xylene solution was spin-coated on the hole injection layer to form a film with a thickness of 20 nm, which was then placed under a nitrogen gas atmosphere and heated on a hot plate at 180° C. for 60 minutes, to form a hole transporting layer.

### (Formation of Light Emitting Layer)

The polymer compound P1 and the metal complex M1 (P1/M1=92% by mass/8% by mass) as a host material were dissolved at a concentration of 2% by mass in xylene as a solvent. The resultant xylene solution was spin-coated on the hole transporting layer to form a film with a thickness of 90 nm, which was then placed under a nitrogen gas atmosphere and heated at 150° C. for 10 minutes, to form a light emitting layer.

### (Formation of Cathode)

The substrate carrying the light emitting layer formed thereon was placed in a vapor deposition machine, and the internal pressure thereof was reduced to 1.0×10⁻⁴ Pa or less, then, sodium fluoride was vapor-deposited with a thickness of about 4 nm on the light emitting layer, then, aluminum was vapor-deposited with a thickness of about 80 nm on the sodium fluoride layer, to form a cathode. After vapor deposition, sealing was performed using a glass substrate, to fabricate a light emitting device D1.

### <Evaluation of Light Emitting Device D1>

The voltage was applied to the light emitting device D1, to observe light emission having the maximum peak of the emission spectrum at 625 nm. The external quantum efficiency at CIE chromaticity coordinate (x, y)=(0.67, 0.33) and 1000 cd/m² was 12.6%. The current value was set so that the current density was 50 mA/cm², then, the device was driven at constant current density and the time change of luminance was measured, to evaluate the luminescence lifetime. The time until the luminance reached 80% of the initial luminance was 128 hours. The results are shown in Table 2 below.

### Example D2

### <Fabrication and Evaluation of Light Emitting Device D2>

A light emitting device D2 was fabricated in the same manner as in Example D1, except that the metal complex M2 was used instead of the metal complex M1.

The voltage was applied to the light emitting device D2, to observe light emission having the maximum peak of the emission spectrum at 610 nm. The external quantum efficiency at CIE chromaticity coordinate (x, y)=(0.65, 0.35) and 1000 cd/m² was 15%. The current value was set so that the current density was 50 mA/cm², then, the device was driven at constant current density and the time change of luminance was measured, to evaluate the luminescence lifetime. The time until the luminance reached 80% of the initial luminance was 24 hours. The results are shown in Table 2 below.

### Comparative Example CD1

### <Fabrication and Evaluation of Light Emitting Device CD1>

A light emitting device CD1 was fabricated in the same manner as in Example D1, except that the metal complex CM1 was used instead of the metal complex M1.

The voltage was applied to the light emitting device CD1, to observe light emission having the maximum peak of the emission spectrum at 590 nm. The external quantum efficiency at CIE chromaticity coordinate (x, y)=(0.57, 0.42) and 1000 cd/m² was 5.4%. The current value was set so that the current density was 50 mA/cm², then, the device was driven at constant current density and the time change of luminance was measured, to evaluate the luminescence lifetime. The time until the luminance reached 80% of the initial luminance was 3 hours. The results are shown in Table 2 below.

### Comparative Example CD2

### <Fabrication and Evaluation of Light Emitting Device CD2>

A light emitting device CD2 was fabricated in the same manner as in Example D1, except that the metal complex CM2 was used instead of the metal complex M1, in Example D1 described above.

The voltage was applied to the light emitting device CD2, to observe light emission having the maximum peak of the emission spectrum at 620 nm. The external quantum efficiency at CIE chromaticity coordinate (x, y)=(0.67, 0.33) and 1000 cd/m² was 3.8%. The current value was set so that the current density was 50 mA/cm², then, the device was driven at constant current density and the time change of luminance was measured, to evaluate the luminescence lifetime. The time until the luminance reached 80% of the initial luminance was 7 hours. The results are shown in Table 2 below.

### Comparative Example CD3

### <Fabrication and Evaluation of Light Emitting Device CD3>

A light emitting device CD3 was fabricated in the same manner as in Example D1, except that the metal complex CM3 was used instead of the metal complex M1, in Example D1 described above.

The voltage was applied to the light emitting device CD3, to observe light emission having the maximum peak of the emission spectrum at 600 nm. The external quantum efficiency at CIE chromaticity coordinate (x, y)=(0.61, 0.39) and 1000 cd/m² was 9.3%. The current value was set so that the current density was 50 mA/cm², then, the device was driven at constant current density and the time change of luminance was measured, to evaluate the luminescence lifetime. The time until the luminance reached 80% of the initial luminance was 13 hours. The results are shown in Table 2 below.

TABLE 2

| | Light | | Luminescence |
|---|---|---|---|
| | emitting | Metal | lifetime |
| | device | complex | (hour) |
| Example D1 | D1 | M1 | 128 |
| Example D2 | D2 | M2 | 24 |
| Comparative | CD1 | CM1 | 3 |
| Example CD1 | | | |
| Comparative | CD2 | CM2 | 7 |
| Example CD2 | | | |
| Comparative | CD3 | CM3 | 13 |
| Example CD3 | | | |

### INDUSTRIAL APPLICABILITY

According to the present invention, a metal complex which is useful for production of a light emitting device showing improved luminescence lifetime can be provided. Further, according to the present invention, a composition containing the metal complex, and a light emitting device containing the metal complex can be provided.

1. A metal complex represented by the following formula (0): 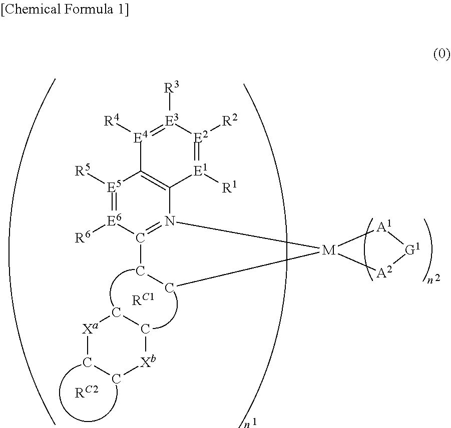
wherein,
M represents a rhodium atom, a palladium atom, an iridium atom or a platinum atom;
n¹ represents 1, 2 or 3; n² represents 0, 1 or 2; n¹+n² is 3 when M is a rhodium atom or an iridium atom, while n¹+n² is 2 when M is a platinum atom or a palladium atom;
when n¹ is 2 or 3, ligands of which number is defined by index n¹ may be the same or different; and when n² is 2, ligands of which number is defined by index n² may be the same or different;
and in respective ligands or one ligand of which number is defined by index n¹, Ring R^{C1} and Ring R^{C2} each independently represent an aromatic hydrocarbon ring or an aromatic heterocyclic ring, and these rings optionally have a substituent, when a plurality of the substituents are present, they may be combined together to form a ring together with atoms to which they are attached;
and in respective ligands or one ligand of which number is defined by index n¹, one of X^{a} and X^{b} is a single bond, the other is a group represented by —C(R^{Xa})₂—, a group represented by —C(R^{Xa})₂—C(R^{Xa})₂— or a group represented by —C(R^{Xa})═C(R^{Xa})—; R^{Xa} represents a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent, when a plurality of the substituents are present, they may be combined together to form a ring together with atoms to which they are attached, a plurality of R^{Xa} may be the same or different and may be combined together to form a ring together with carbon atoms to which they are attached;
and in respective ligands or one ligand of which number is defined by index n¹, E¹, E², E³, E⁴, E⁵, and E⁶ each independently represent a nitrogen atom or a carbon atom, and at least E¹ and E², E² and E³, E³ and E⁴, E⁴ and E⁵, or E⁵ and E⁶, are carbon atoms;
provided that, when E¹ is a nitrogen atom, R¹ is not present; when E² is a nitrogen atom, R² is not present; when E³ is a nitrogen atom, R³ is not present; when E⁴ is a nitrogen atom, R⁴ is not present; when E⁵ is a nitrogen atom, R⁵ is not present; when E⁶ is a nitrogen atom, R⁶ is not present;
and in respective ligands or one ligand of which number is defined by index n¹, the combination of one of R¹ and R², R² and R³, R³ and R⁴, R⁴ and R⁵, and R⁵ and R⁶ is integrated to form a group represented by the following formula (P); R¹, R², R³, R⁴, R⁵ and R⁶ not forming a group represented by the following formula (P) each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent, when a plurality of the substituents are present, they may be combined together to form a ring together with atoms to which they are attached; and in respective ligands or one ligand of which number is defined by index n¹, when a plurality of R¹, R², R³, R⁴, R⁵ and R⁶ not forming a group represented by the following formula (P) are present, they may be the same or different at each occurrence, and may be combined together to form a ring together with carbon atoms to which they are attached;
and in respective ligands or one ligand of which number is defined by index n², A¹-G¹-A² represents an anionic bidentate ligand, G¹ represents an atomic group constituting a bidentate ligand together with A¹ and A², A¹ and A² each independently represent a carbon atom, an oxygen atom or a nitrogen atom, or an atomic group having them, and these atomic groups may be ring-constituent atomic groups, 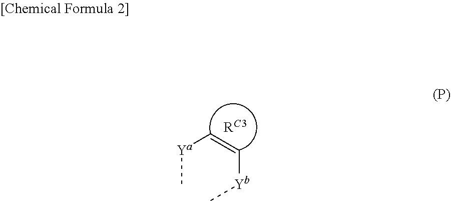
wherein,
the dotted line denotes a bond to E¹, E², E³, E⁴, E⁵ or E⁶;
Ring R^{C3} represents an aromatic hydrocarbon ring or an aromatic heterocyclic ring, and these rings optionally have a substituent, when a plurality of the substituents are present, they may be combined together to form a ring together with atoms to which they are attached;
one of Y^{a} and Y^{b} is a single bond, and the other is a group represented by —C(R^{Ya})₂—, R^{Ya} represents a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent, when a plurality of the substituents are present, they may be combined together to form a ring together with atoms to which they are attached; a plurality of R^{Ya} may be the same or different and may be combined together to form a ring together with carbon atoms to which they are attached.

2. The metal complex according to claim 1, wherein in respective ligands or one ligand of which number is defined by index n¹ in said formula (0), R² and R³ are integrated to form a group represented by said formula (P).

3. The metal complex according to claim 1, wherein said formula (P) is represented by the following formula (P′): 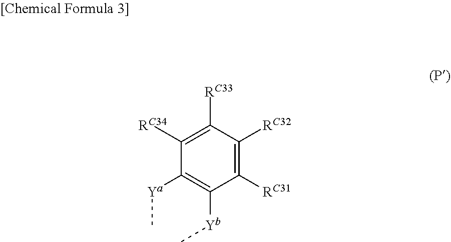
wherein,
the dotted line denotes a bond to E¹, E², E³, E⁴, E⁵ or E⁶;
Y^{a} and Y^{b} represent the same meaning as for Y^{a} and Y^{b} in said formula (P), respectively;
R^{C31}, R^{C32}, R^{C33} and R^{C34} each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent; R^{C31} and R^{C32}, R^{C32} and R^{C33}, and R^{C33} and R^{C34} each may be combined together to form a ring together with atoms to which they are attached.

4. The metal complex according to claim 3, wherein the metal complex represented by said formula (0) is a metal complex represented by the following formula (2-A1), the following formula (2-B1) or the following formula (2-C1): 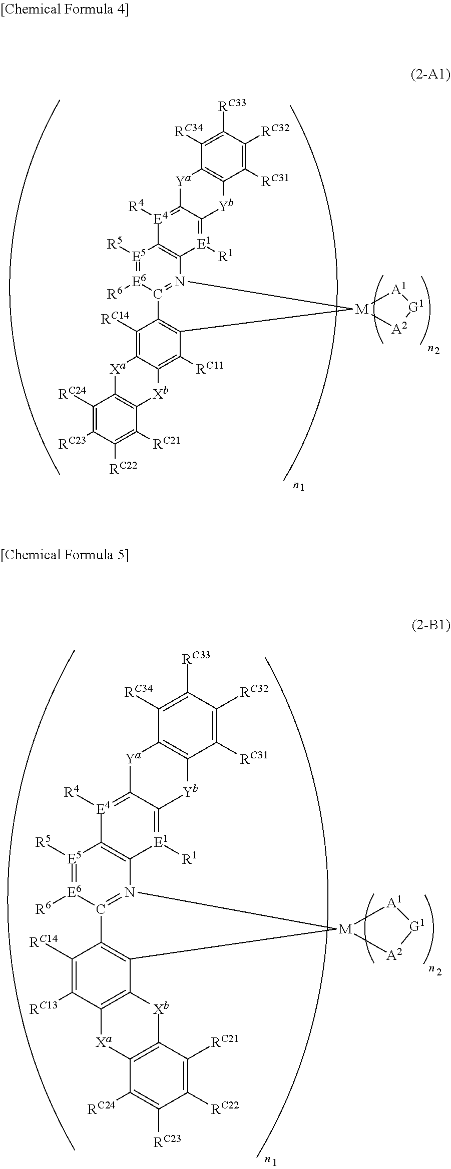 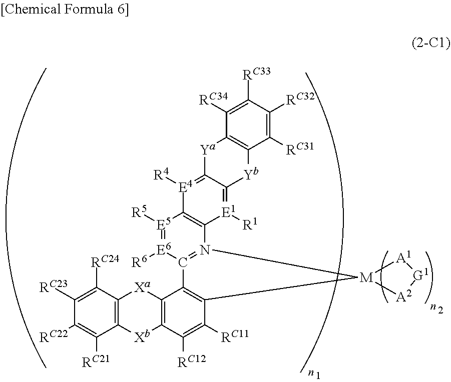
in the formula (2-A1), the formula (2-B1), and the formula (2-C1),
M, n¹, n², X^{a}, X^{b}, E¹, E⁴, E⁵, E⁶, R¹, R⁴, R⁵, R⁶, and A¹-G¹-A² represent the same meaning as for M, n¹, n², X^{a}, X^{b}, E¹, E⁴, E⁵, E⁶, R¹, R⁴, R⁵, R⁶, and A¹-G¹-A² in said formula (0), respectively; Y^{a} and Y^{b} represent the same meaning as for Y^{a} and Y^{b} in said formula (P), respectively; R^{C31}, R^{C32}, R^{C33}, and R^{C34} represent the same meaning as for R^{C3}1, R^{C32}, R^{C33}, and R^{C34} in said formula (P′), respectively;
R^{C11} and R^{C14} in the formula (2-A1), R^{C13} and R^{C14} in the formula (2-B1), and R^{C11} and R^{C12} in the formula (2-C1) each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent;
R^{C21}, R^{C22}, R^{C23} and R^{C24} each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent; and in respective ligands or one ligand of which number is defined by index n¹, R^{C11} and R^{C12}, R^{C13} and R^{C14}, R^{C21} and R^{C22}, R^{C22} and R^{C23}, and R^{C23} and R^{C24} each may be combined together to form a ring together with atoms to which they are attached.

5. The metal complex according to claim 4, wherein the metal complex represented by said formula (2-A1) is a metal complex represented by the following formula (2-A1-1): 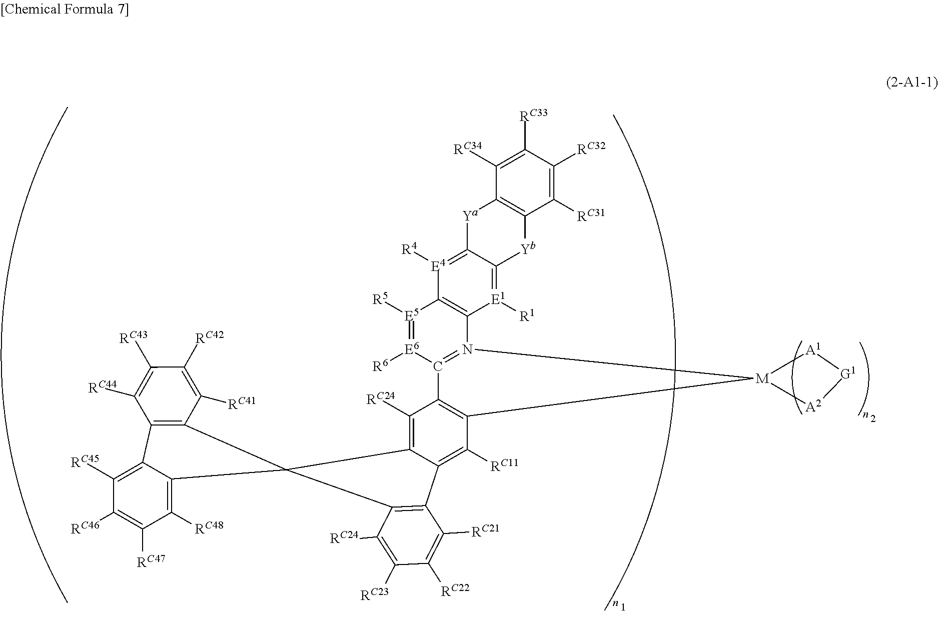
wherein,
M, n¹, n², E¹, E⁴, E⁵, E⁶, R¹, R⁴, R⁵, R⁶, and A¹-G¹-A² represent the same meaning as for M, n¹, n², E¹, E⁴, E⁵, E⁶, R¹, R⁴, R⁵, R⁶, and A¹-G¹-A² in said formula (0), respectively; Y^{a} and Y^{b} represent the same meaning as for Y^{a} and Y^{b} in said formula (P), respectively; R^{C31}, R^{C32}, R^{C33}, and R^{C34} represent the same meaning as for R^{C31}, R^{C32}, R^{C33}, and R^{C34} in said formula (P′), respectively; R^{C11}, R^{C14}, R^{C21}, R^{C22}, R^{C23}, and R^{C24} represent the same meaning as for R^{C11}, R^{C14}, R^{C21}, R^{C22}, R^{C23}, and R^{C24} in said formula (2-A1), respectively;
R^{C41}, R^{C42}, R^{C43}, R^{C44}, R^{C45}, R^{C46}, R^{C47} and R^{C48} each independently represent a hydrogen atom, an alkyl group, a cycloalkyl group, an alkoxy group, a cycloalkoxy group, an aryl group, an aryloxy group, a monovalent heterocyclic ring group, a substituted amino group or a halogen atom, and these groups optionally have a substituent; and in respective ligands or one ligand of which number is defined by index n¹, R^{C41} and R^{C42}, R^{C42} and R^{C43}, R^{C43} and R^{C44}, R^{C45} and R^{C46}, R^{C46} and R^{C47}, and R^{C47} and R^{C48} each may be combined together to form a ring together with atoms to which they are attached.

6. The metal complex according to claim 1, wherein said Ring R^{C1}, said Ring R^{C2} or said Ring R^{C3} has a group represented by the following formula (D-A), the following formula (D-B) or the following formula (D-C) as a substituent: 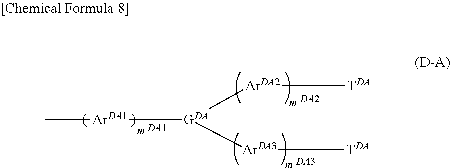
wherein,
m^{DA1} m^{DA2} and m^{DA3} each independently represent an integer of 0 or more;
G^{DA} represents a nitrogen atom, an aromatic hydrocarbon group or a heterocyclic ring group, and these groups optionally have a substituent;
Ar^{DA1}, Ar^{DA2} and Ar^{DA3} each independently represent an arylene group or a divalent heterocyclic ring group, and these groups optionally have a substituent; when a plurality of Ar^{DA1}, Ar^{DA2} and Ar^{DA3} are present, they may be the same or different at each occurrence;
T^{DA} represents an aryl group or a monovalent heterocyclic ring group, and these groups optionally have a substituent; a plurality of T^{DA} may be the same or different, 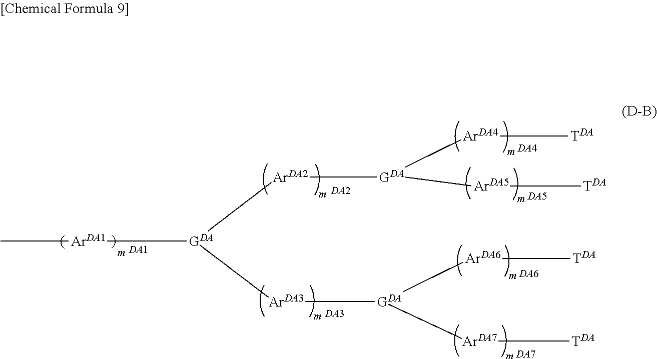
wherein,
m^{DA1}, m^{DA2}, m^{DA3}, m^{DA4}, m^{DA5}, m^{DA6} and m^{DA7} each independently represent an integer of 0 or more;
G^{DA} represents a nitrogen atom, an aromatic hydrocarbon group or a heterocyclic ring group, and these groups optionally have a substituent; a plurality of G^{DA} may be the same or different;
Ar^{DA1}, Ar^{DA2}, Ar^{DA3}, Ar^{DA4}, Ar^{DA5}, Ar^{DA6} and Ar^{DA7} each independently represent an arylene group or a divalent heterocyclic ring group, and these groups optionally have a substituent; when a plurality of Ar^{DA1}, Ar^{DA2}, Ar^{DA3}, Ar^{DA4}, Ar^{DA5}, Ar^{DA6} and Ar^{DA7} are present, they may be the same or different at each occurrence;
T^{DA} represents an aryl group or a monovalent heterocyclic ring group, and these groups optionally have a substituent; a plurality of T^{DA} may be the same or different, 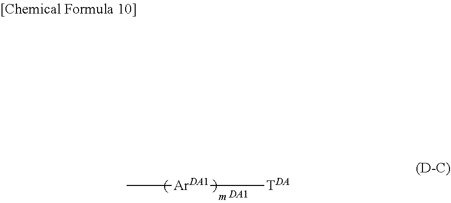
wherein,
m^{DA1} represents an integer of 0 or more;
Ar^{DA} represents an arylene group or a divalent heterocyclic ring group, and these groups optionally have a substituent; when a plurality of Ar^{DA1} are present, they may be the same or different;
T^{DA} represents an aryl group or a monovalent heterocyclic ring group, and these groups optionally have a substituent.

7. The metal complex according to claim 1, wherein R¹, R², R³, R⁴, R⁵ or R⁶ not forming a group represented by said formula (P) is a group represented by said formula (D-A), said formula (D-B) or said formula (D-C).

8. The metal complex according to claim 7, wherein R⁴ or R⁵ not forming a group represented by said formula (P) is a group represented by said formula (D-A), said formula (D-B) or said formula (D-C).

9. The metal complex according to claim 1, wherein said M is an iridium atom, and said n¹ is 3.

10. A composition comprising
the metal complex as described in claim 1, and
at least one material selected from the group consisting of a host material, a light emitting material other than said metal complex, an antioxidant and a solvent.

11. The composition according to claim 10, wherein said host material contains at least one of a low molecular compound represented by the following formula (H-1) and a polymer compound containing a constitutional unit represented by the following formula (Y): 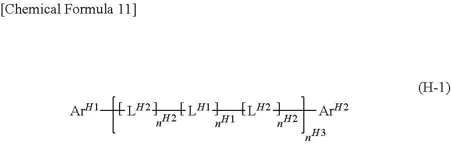
wherein,
Ar^{H1} and Ar^{H2} each independently represent an aryl group or a monovalent heterocyclic ring group, and these groups optionally have a substituent;
n^{H1} and n^{H2} each independently represent 0 or 1; when a plurality of n^{H1} are present, they may be the same or different; when a plurality of n^{H2} are present, the plurality of n^{H2} may be the same or different;
n^{H3} represents an integer of 0 or more;
L^{H1} represents an arylene group, a divalent heterocyclic ring group, or a group represented by —[C(R^{H11})₂]n^{H11}-, and these groups optionally have a substituent, when a plurality of L^{H1} are present, they may be the same or different, n^{H11} represents an integer of 1 or more and 10 or less; R^{H11} represents a hydrogen atom, an alkyl group, a cycloalkyl group, an alkoxy group, a cycloalkoxy group, an aryl group or a monovalent heterocyclic ring group, and these groups optionally have a substituent, a plurality of R^{H11} may be the same or different and may be combined together to form a ring together with carbon atoms to which they are attached;
L^{H2} represents a group represented by —N(-L^{H21}-R^{H21})—; when a plurality of L^{H2} are present, the plurality of L^{H2} may be the same or different; L^{H21} represents a single bond, an arylene group or a divalent heterocyclic ring group, and these groups optionally have a substituent, R^{H21} represents a hydrogen atom, an alkyl group, a cycloalkyl group, an aryl group or a monovalent heterocyclic ring group, and these groups optionally have a substituent, 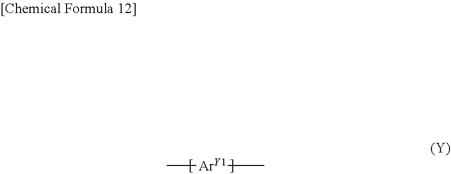
wherein, Ar^{Y1} represents an arylene group, a divalent heterocyclic ring group, or a divalent group in which an arylene group and a divalent heterocyclic ring group are bonded directly, and these groups optionally have a substituent.

12. A light emitting device comprising the metal complex as described in claim 1.
